(19) United States
(12) Patent Application Publication
(10) Pub. No.: US 2002/0068354 A1
(43) Pub. Date: Jun. 6, 2002

(54) Feline immunodeficiency virus gene therapy vectors

(75) Inventors: Julie C. Johnston, Hockessin, DE (US); Sybille L. Sauter, Del Mar, CA (US); David Chi-Tang Hsu, San Diego, CA (US); Philip Lee Sheridan, San Diego, CA (US); Stephen F. Hardy, San Francisco, CA (US); Thomas W. Dubensky JR., Piedmont, CA (US); Jiing-Kuan Yee, Arcadia, CA (US)

Correspondence Address: CHIRON CORPORATION, Intellectual Property - R 440, P. O. Box 8097, Emeryville, CA, 94662-8097, US

(73) Assignee: CHIRON CORPORATION

(21) Appl. No.: 09/797,518

(22) Filed: Mar. 1, 2001

Related U.S. Application Data
(63) Continuation-in-part of application No. 09/231,235, filed on Jan. 15, 1999.
(60) Provisional application No. 60/071,731, filed on Jan. 16, 1998.
(60) Provisional application No. 60/086,825, filed on May 26, 1998.
(60) Provisional application No. 60/114,955, filed on Jan. 4, 1999.

(51) Int. Cl. C12N 7/01; C12N 15/867; C07H 21/04; C07K 1/00; C12N 15/74; C12N 15/63

(52) U.S. Cl. 435/235.1; 435/456; 530/826; 536/23.72; 536/24.1; 435/320.1

(57) ABSTRACT

Disclosed are gene therapy vectors based upon the feline immunodeficiency virus, as well as related packaging cell lines, methods for production, and methods of use.

### CROSS-REFERENCES TO RELATED APPLICATIONS

The present application is a continuation-in-part of U.S. application Ser. No. 09/231,235, filed Jan. 15, 1999, which claims priority from U.S. Provisional Application No. 601071,731, filed Jan. 16, 1998, U.S. Provisional Application No. 60/086,825, filed May 26, 1998, and U.S. Provisional Application No. 60/114,955, filed Jan. 4, 1999, which applications are hereby incorporated by reference in their entirety.

### TECHNICAL FIELD

The present invention relates generally to pharmaceutical compositions and methods, and more particularly, to feline immunodeficiency virus gene therapy vectors which are suitable for a wide variety of therapeutic and research applications.

### BACKGROUND OF THE INVENTION

Since the discovery of nucleic acids in the 1940's and continuing through the most recent era of biotechnology, substantial research has been undertaken in order to realize the possibility that the course of disease may be affected through interaction with the nucleic acids of living organisms. Most recently, a wide variety of methods have been described for altering or affecting genes, including for example, viral vectors derived from retroviruses, adenoviruses, vaccinia viruses, herpes viruses, and adeno-associated viruses (see Jolly, Cancer Gene Therapy 1(1):51-64, 1994).

Of these techniques, recombinant retroviral gene delivery methods have been most extensively utilized, in part due to: (1) the efficient entry of genetic material (the vector genome) into cells; (2) an active, efficient process of entry into the target cell nucleus; (3) relatively high levels of gene expression; (4) the potential to target particular cellular subtypes through control of the vector-target cell binding and the tissue-specific control of gene expression; (5) a general lack of pre-existing host immunity; (6) substantial knowledge and clinical experience which has been gained with such vectors; and (7) the capacity for stable and long-term expression.

Briefly, retroviruses are diploid positive-strand RNA viruses that replicate through an integrated DNA intermediate. In particular, upon infection by the RNA virus, the retroviral genome is reverse-transcribed into DNA by a virally encoded reverse transcriptase that is carried as a protein in each retrovirus. The viral DNA is then integrated pseudo-randonmly into the host cell genome of the infected cell, forming a “provirus” which is inherited by daughter cells.

One type of retrovirus, the murine leukemia virus, or “MLV”, has been widely utilized for gene therapy applications (see generally Mann et al. Cell 33:153, 1983; Cane and Mulligan, PNAS 81:6349, 1984; and Miller et al., Human Gene Therapy 1:5-14, 1990). One major disadvantage of MLV-based vectors, however, is that the host range (i.e., cells infected with the vector) is limited, and the frequency of transduction of non-replicating cells is generally low.

The present invention provides a new gene therapy delivery vehicle based in part upon the feline immunodeficiency virus, as well as related packaging cell lines. The invention also provides other, related, advantages.

### SUMMARY OF THE INVENTION

Briefly stated, the present invention provides gene therapy delivery vehicles which are based upon feline immunodeficiency viruses (“FIV”). The genome organization of FIV, a lentivirus, is shown in FIG. 1. FIV differs from the simple oncoretrovirus, MLV, as well as the more complex lentiviruses HIV-1 and HIV-2, in the number of accessory proteins it encodes, its host range, mode of transmission and pathogenesis.

Within one aspect, FIV vectors are provided comprising a 5′ FIV LTR, a primer binding site (e.g., a tRNA binding site), one or more heterologous sequences operably linked to a promoter element (which may be the 5′ FIV LTR, a hybrid LTR, non-FIV promoter, or a combination of these), an origin of second strand DNA synthesis and a 3′ FIV LTR. As utilized herein, “promoters” can include pol I, pol II and pol III promoters. Within preferred embodiments, the FIV vectors may further comprise a packaging signal (which can be an FIV packaging signal, heterologous retroviral packaging signal, a synthesized signal, or any combination of these).

Within one embodiment the promoter is a tissue specific promoter, or alternatively, a promoter such as CMV, PGK or RSV. Within further embodiments, the FIV vector further comprises an internal ribosome entry site. In still further embodiments, the promoter may be operably linked to two genes of interest which are separated by less than 120 nucleotides. Within other embodiments, the vector has at least one RNA export element selected from the group consisting of MPMV, HBV, RSV and lentiviral Rev-responsive-elements. Within yet other embodiments the FIV LTR is composed of less than 85%, 75%, 60%, 55%, 50%, or 45% wild type FIV LTR nucleic acid sequence, and/or FIV LTR contains at least one non-FIV promoter or promoter/enhancer.

Within various embodiments, the FIV vector expresses a gene of interest. Representative examples of suitable genes of interest include marker genes, cytokines, factor VIII, factor IX, LDL receptor, prodrug activating enzymes, β-glucuronidase, CFTR, sarco-glycans, angiogenesis promoting and preventing factors (e.g., FGF-2 and 16K, respectively), glucokinase, growth factors, trans-dominant negative viral or cancer-associated proteins, and tyrosine hydroxylase.

Within other aspects of the invention, packaging expression cassettes are provided comprising a promoter operably linked to a sequence encoding FIV gag/pol or parts thereof The FIV gag/pol polyprotein organization, as well as that of HIV is depicted in FIG. 2. Unlike HIV, FIV encodes a dUTPase upstream of the integrase, which may or may not be included or functional in the FIV packaging expression cassettes. Within other embodiments, the cassettes further comprises one or more sequence elements selected from the group consisting of vif, ORF 2 or rev. Within further embodiments, said packaging expression cassettes comprise at least one RNA export element.

Within another aspect, rev expression cassettes are provided comprising a promoter operably linked to a sequence selected from at least one of vif, rev or ORF 2. Within a related aspect, expression cassettes may further include a promoter operably linked to an envelope sequence of viral origin. Example embodiments include expression cassettes containing env sequences from VSV (Vesicular Stomatitis Virus); amphotropic, ecotropic, polytropic or xenotropic MLV-derived; HIV, FIV, FeLV, SNV, alpha virus, and GaLV (Gibbon Ape Leukemia Virus). Preferred embodiments are expression cassettes wherein the env sequence encodes an amphotropic envelope. In one preferred embodiment, a VSV-G expression cassette is provided containing a promoter operably linked to a sequence encoding VSV-G env gene.

In a related aspect, this invention includes host cells (e.g. of human, dog, cat or murine origin) which contain any of the aforementioned expression cassettes.

Within further aspects, packaging cell lines are provided comprising an expression cassette comprising a promoter operably linked to a sequence encoding FIV gag/pol (including dUTPase) and an RNA export element, an expression cassette comprising a promoter operably linked to a sequence encoding an envelope, wherein said promoter is operably linked to said sequence encoding gag/pol. Within further embodiments, the packaging cell line further comprises a sequence encoding one or more of vif, rev or ORF 2. Within preferred embodiments, the expression cassette is stably integrated within the cell, and/or upon introduction of a FIV vector construct, produces particles at a concentration of greater than 10³ cfu/ml. Within preferred embodiments, the promoter is inducible. Within other embodiments, an expression cassette facilitating the regulation of other expression cassettes necessary for an FIV PCL or VPCL, is introduced. Within other embodiments, the envelope is VSV-G. Within preferred embodiments of the invention, the packaging cell lines, upon introduction of a FIV vector, produce particles that are free of replication competent virus.

These and other aspects of the present invention will become evident upon reference to the following detailed description and attached drawings. In addition, various references are set forth below which describe in more detail certain procedures or compositions (e.g., plasmids, etc.), and are therefore incorporated by reference in their entirety as if each were individually noted for incorporation.

### BRIEF DESCRIPTION OF THE DRAWINGS

FIG. 1 schematically illustrates the genomic organization of FIV.

FIG. 2 schematically illustrates the organization of gag/pol polyproteins of FIV.

FIGS. 3 schematically illustrates a variety of different FIV vector and packaging constructs.

FIG. 4 schematically illustrates the genomic organization of FIV and FIVΔEnv Proviral DNA.

FIGS. 5A-B graphically depicts FIV vector transduction efficiencies in the absence of cytokine stimulation were comparable to those in cells that were prestimulated with cytokines

FIG. 5C graphically depicts eGFP expression in the recipient mice.

FIG. 5D graphically depicts different cell types expressing eGFP: granulocyte, monocyte and lymphocyte populations based on parameters of forward side scatter (FSC) and side side scatter (SSC).

FIG. 6A graphically depicts FIV transduced human HPCs.

FIG. 6B graphically depicts FIV-based vector transduce human CD34⁺ cells in a dose-dependent manner.

FIG. 6C graphically depicts eGFP⁺ cells maintaining the CD34⁺ phenotype.

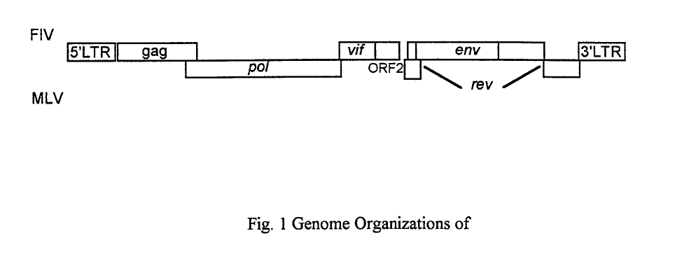

### DETAILED DESCRIPTION OF THE INVENTION

Prior to setting forth the invention, it may be helpful to an understanding thereof to first set forth definitions of certain terms that will be used hereinafter.

“FIV retroviral vector construct”, “FIV vector,” and “recombinant FIV vector” refers to a nucleic acid construct which carries, and within certain embodiments of the invention, is capable of directing the expression of a sequence(s) or gene(s) of interest. Briefly, the FIV vector includes at least one transcriptional promoter or promoter/enhancer or locus defining element(s), or other elements which control gene expression by other means such as alternative splicing, RNA export, post-translational modification of messenger, or post-transcriptional modification of protein. Such vector constructs may also include a packaging signal, long terminal repeats (LTRs) or portion thereof, and positive and negative strand primer binding sites. Optionally, the recombinant FIV vector may also include a signal which directs polyadenylation, selectable and/or non-selectable markers, an origin of second strand DNA synthesis, as well as one or more restriction sites and a translation termination sequence. Examples for markers include, but are not limited to, neomycin (Neo), thymidin kinase (TK), hygromycin, phleomycin, puromycin, histidinol, green fluorescent protein (GFP), human placental alkaline phosphatase (PLAP), DHFR, β-galactosidase and human growth hormone (hGH). By way of example, such vectors typically include a 5′ FIV LTR, a primer binding site, a packaging signal, an origin of second strand DNA synthesis, and a 3′ FIV LTR.

A wide variety of heterologous sequences may be included within the vector construct, including for example, sequences which encode a protein (e.g., cytotoxic protein, disease-associated antigen, immune accessory molecule, or replacement gene), or which are useful as a molecule itself (e.g., as a ribozyme or antisense sequence). Alternatively, the heterologous sequence may merely be a “stuffer” or “filler” sequence, which is of a size sufficient to allow production of viral particles containing the RNA genome.

“Primer binding site” refers to a sequence which is at least partially complementary to another nucleic acid molecule which binds to and initiates the synthesis of a daughter strand. Representative examples include a tRNA binding site, small nuclear RNA binding sites, pre-RNA binding sites and the like.

“Expression cassette” refers to an assembly, which is capable of directing the expression of the sequence(s) or gene(s) of interest. The expression cassette includes a promoter or promoter/enhancer which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include a bacterial origin of replication, one or more selectable markers, a signal which allows the plasmid construct to exist as single-stranded DNA (e.g., a M13 origin of replication), at least one multiple cloning site, and a “mammalian” origin of replication (e.g., a SV40 or adenovirus origin of replication).

“Packaging cell” refers to a cell, which contains those elements necessary for production of infectious recombinant retrovirus which are lacking in a recombinant retroviral vector. Typically, such packaging cells contain one or more expression cassettes which are capable of expressing proteins which encode gag, pol and env-derived proteins. Packaging cells can also contain expression cassettes encoding one or more of vif, rev, or ORF 2 in addition to gag/pol and env expression cassettes.

“Producer cell” or “Vector Producing Cell Line” (VCL) refers to a cell which contains all elements necessary for production of recombinant FIV vector particles.

“FIV vector particle” as utilized within the present invention refers to a viral particle which carries at least one gene or nucleotide sequence of interest, and may also contain a selectable marker. The recombinant FIV particle is capable of reverse transcribing its genetic material into DNA and incorporating this genetic material into a host cell's DNA upon transduction. FIV vector particles may have a lentiviral envelope, a non-lentiviral envelope (e.g., an amphotropic or VSV-G envelope), a chimeric envelope or a modified envelope (e.g., truncated envelopes, envelopes containing hybrid sequences or synthetic sequences).

### CONSTRUCTION OF FIV VECTOR CONSTRUCTS

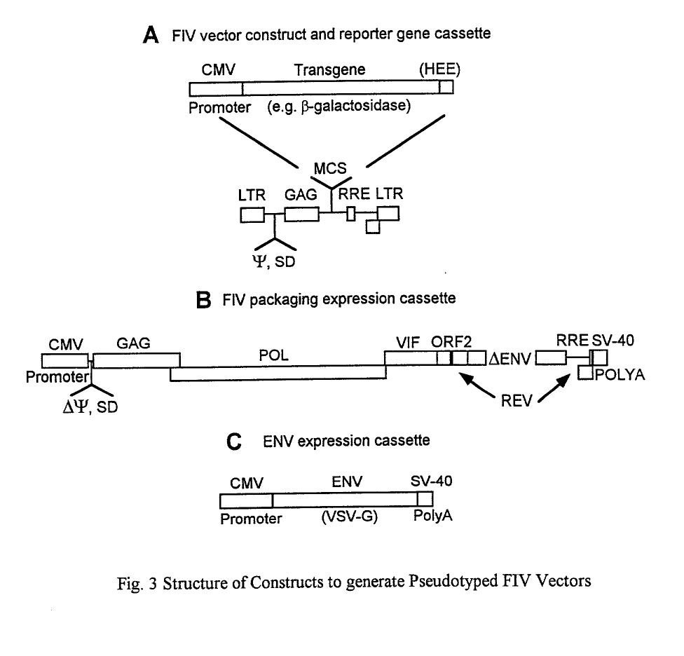

As noted above, the present invention provides FIV vectors, which are designed to carry or express a selected gene(s) or sequence(s) of interest. Briefly, FIV vectors may be readily constructed from a wide variety of FIV strains. Representative examples of FIV strains and molecular clones of such isolates include the Petaluma strain and its molecular clones FIV34TF10 and FIV14 (Olmsted et al., PNAS 86:8088-8092, 1989; Olmsted et al., PNAS 86:2448-2452, 1989; Talbot et al., PNAS 86:5743-5747, 1989), the San Diego strain and its molecular clone PPR (Phillips et al., J. Virology 64:4605-4613, 1990), the Japanese strains and their molecular clones FTM191CG and FIV-TM2 (Miyazawa et al., J. Virology 65:1572-1577, 1991) and the Amsterdam strain and its molecular clone 19K1 (Siebelink et al., J. Virology 66:1091-1097, 1992). Such FIV strains may either be obtained from feline isolates, or more preferably, from depositories or collections such as the American Type Culture Collection (ATCC, Rockville, Md.), or isolated from known sources using commonly available techniques. Representative examples of such FIV vector constructs are set forth in more detail below and are shown in FIG. 3.

Any of the above FIV strains may be readily utilized in order to assemble or construct FIV gene delivery vehicles given the disclosure provided herein, and standard recombinant techniques (e.g., Sambrook et al., Molecular Cloning. A laboratory Manual, 2nd ed., Cold Spring Harbor Laboratory Press, 1989; Kunkel, PNAS 82:488, 1985). In addition, within certain embodiments of the invention, portions of the FIV gene delivery vehicles may be derived from different viruses. For example, within one embodiment of the invention, recombinant FIV vector or LTR sequences may be partially derived or obtained from HIV-1, a packaging signal from SIV, and an origin of second strand synthesis from HV-2.

Within one aspect of the present invention, FIV vector constructs are provided comprising a 5′ FIV LTR, a primer binding site, one or more heterologous sequences, an origin of second strand DNA synthesis, and a 3′ FIV LTR. Briefly, Long Terminal Repeats (“LTRs”) are subdivided into three elements, designated U5, R and U3. These elements contain a variety of signals, which are responsible for the biological activity of a retrovirus, including for example, promoter and enhancer elements, which are located within U3. LTRs may be readily identified in the provirus (integrated DNA form) due to their precise duplication at either end of the genome. As utilized herein, a 5′ FIV LTR should be understood to include a 5′ promoter or promoter/enhancer element to allow reverse transcription and integration of the DNA form of the vector. The 3′ FIV LTR should be understood to include a polyadenylation signal to allow reverse transcription and integration of the DNA form of the vector.

The 3′ FIV LTR may be mutated in its putative initiator sequence (at the RNA cap site) (see, e.g., Ikeda et al., Virus Research 51:203-212, 1997).

Within one aspect of the invention, FIV vector constructs are provided which contain hybrid FIV LTRs where up to 45% of the wildtype FIV LTR sequence is deleted and replaced by one or more viral or non-viral promoter or promoter/enhancer elements (e.g., other retroviral LTRs and/or non-retroviral promoters or promoter/enhancers such as the CMV promoter/enhancer or the SV40 promoter) similar to the hybrid LTRs described by Chang, et al., J. Virology 67, 743-752, 1993; Finer, et al., Blood 83, 43-50, 1994 and Robinson, et al., Gene Therapy 2, 269-278, 1995.

The tRNA binding site and origin of second strand DNA synthesis are also important for a retrovirus to be biologically active, and may be readily identified by one of skill in the art. For example, tRNA binds to a retroviral tRNA binding site by Watson-Crick base pairing, and is carried with the retrovirus genome into a viral particle. The tRNA is then utilized as a primer for DNA synthesis by reverse transcriptase. The tRNA binding site may be readily identified based upon its location just downstream from the 5′ LTR. Similarly, the origin of second strand DNA synthesis is, as its name implies, important for the second strand DNA synthesis of a retrovirus. This region, which is also referred to as the poly-purine tract, is located just upstream of the 3′ LTR.

The packaging signal sequence of FIV directs packaging of viral genetic material into the viral particle. A major part of the packaging signal in FIV lies between the 5′ FIV LTR and the gag/pol sequence with the packaging signal likely overlapping in part with the 5′ area of the gag/pol sequence. The exact location and size of a minimal FIV packaging signal is not yet identified.

In addition to 5′ and 3′ FIV LTRs, a tRNA binding site, a packaging signal, and an origin of second strand DNA synthesis, certain preferred recombinant FIV vector constructs which are provided herein also comprise one or more genes of interest, each of which is discussed in more detail below. In addition, the FIV vectors may include one or more RNA export elements (also variously referred to as RNA transport, nuclear transport or nuclear export elements) which, within one aspect of the invention is the FIV RRE (Rev-responsive element). In other embodiments, the FIV vectors do not contain RNA export elements. Within another aspect of the invention, the RNA export element is not FIV RRE but a heterologous transport element. Representative examples of suitable heterologous RNA export elements include the Mason-Pfizer monkey virus constitutive transport element, the MPMV CTE (Bray et al., PNAS USA 91:1256-1260, 1994), the Hepatitis B Virus posttranscriptional regulatory element, the IBV PRE (Huang et al., Mol. Cell. BioL 13:7476-7486, 1993 and Huang et al., J. Virology 68:3193-3199, 1994), other lentiviral Rev-responsive elements (Daly et al., Nature 342:816-819, 1989 and Zapp et al., Nature 342:714-716, 1989) or the PRE element from the woodchuck hepatitis virus. Further RNA export elements include the element in Rous sarcoma virus (Ogert et al., J. Virology 70:3834-3843, 1996; Liu & Mertz, Genes & Dev. 9:1766-1789, 1995) and the element in the genome of simian retrovirus type 1 (Zolotukhin et al., J. Virology 68:7944-7952, 1994). Other potential elements include the elements in the histone gene (Kedes, Annu. Rev. Biochem. 48:837-870, 1970), the 0a interferon gene (Nagata et al., Nature 287:401-408, 1980), the 0-adrenergic receptor gene (Koilka et al., Nature 329:75-79, 1987), and the c-Jun gene (Hattorie et al., PNAS 85:9148-9152, 1988).

Within one aspect of the invention, recombinant FIV vector constructs are provided which contain one or more multiple cloning sites and/or code for one or more marker genes such as the ones described above.

Within one aspect of the invention, recombinant FIV vector constructs are provided which lack both gag/pol and env coding sequences. As utilized herein, the phrase “lacks gag/pol or env coding sequences” should be understood to mean that the FIV vector contains less than 20, preferably less than 15, more preferably less than 10, and most preferably less than 8 consecutive nucleotides which are found in gag/pol or env genes, and in particular, within gag/pol or env expression cassettes that are used to construct packaging cell lines for the FIV vector construct. This aspect of the invention provides for FIV vectors having a low probability of undesirable recombination with gag/pol or env sequences which may occur in a host cell or be introduced therein, for example, by transformation with an expression cassette. The production of FIV vector constructs lacking gag/pol or env sequences may be accomplished by partially eliminating the packaging signal and/or the use of a modified or heterologous packaging signal. Within other embodiments of the invention, FIV vector constructs are provided wherein a portion of the packaging signal that may extend into, or overlap with, the FIV gag/pol sequence is modified (e.g., deleted, truncated or bases exchanged). Within other aspects of the invention, FIV vector constructs are provided which include the packaging signal that may extend beyond the start of the gag/pol gene. Within certain embodiments, the packaging signal that may extend beyond the start of the gaglpol gene is modified in order to contain one, two or more stop codons within the gag/pol reading frame. Most preferably, one of the stop codons eliminates the gag/pol start site. In other embodiments, the introduced mutation may cause a frame shift in the gag/pol coding region.

Within certain embodiments of the invention, FIV vectors are provided wherein viral promoters, preferably CMV or RSV promoters and/or and or cellular protors such as PGK and/or enhancers are utilized to drive expression of one or more genes of interest.

Within other aspects of the invention, FIV vectors are provided wherein tissue-specific promoters are utilized to drive expression of one or more genes of interest. For example, FIV vectors of the invention can contain a liver specific promoter to maximize the potential for liver specific expression of the exogenous DNA sequence contained in the vectors. Preferred liver specific promoters include the hepatitis B X-gene promoter and the hepatitis B core protein promoter. These liver specific promoters are preferably employed with their respective enhancers. See also PCT Patent Publications WO 90/07936 and WO 91/02805 for a description of the use of liver specific promoters in FIV vector particles.

Within certain embodiments of the invention, the FIV vector constructs provided herein may be generated such that more than one gene of interest is expressed. This may be accomplished through the use of di- or oligo-cistronic cassettes (e.g., where the coding regions are separated by 120 nucleotides or less, see generally Levin et al., Gene 108:167-174, 1991), or through the use of Internal Ribosome Entry Sites (“IRES”).

Within one aspect of the invention, self-inactivating (SIN) vectors are made by deleting and/or altering promoter and enhancer elements in the U3 region of the 3′LTR, including the TATA box. The deletion is transferred to the 5′LTR after reverse transcription and integration in transduced cells. This results in the transcriptional inactivation of the LTR in the provirus.

Possible advantages of SIN vectors include increased safety of the gene delivery system by 1) decreasing the chance of insertional activation of cellular oncogenes, 2) preventing the generation of replication-competent virus, and 3) possibly providing tighter regulation of the gene of interest from internal promoters. Furthermore, inactivation of the FIV LTR may potentially reduce promoter interference between the LTR and the internal promoter thereby resulting in enhanced expression of the transgene. In addition, tighter control of regulatable gene therapy vectors is possible due to the lack of an upstream promoter element in the 5′LTR.

Putative enhancer elements are deleted in combination with a dinucleotide alteration in the TATA box to inactivate the FIV promoter (Examples 15A, 15B, 15D, 15E). However, one shortcoming has been the observation that murine based SIN vectors have reduced titers compared to standard retroviral vectors (Yu et al., Proc. Natl. Acad. Sci. 83: 3194, 1986; Yee et al., Proc. Natl. Acad. Sc. 84: 5197, 1987). Therefore, to minimize the negative impact on viral titers, an additional aspect of the invention will include a more limiting and specific mutational approach designed to target putative transcription factor binding sites (such as those for ATF-1 and AP-1; Elder and Phillips, Adv. In Virus Res. 45: 275, 1995; Ikeda et al., J. Gen Virol. 79: 95, 1998). Examples are described for constructing SIN vectors which target specific transcriptional regulatory sequences while deleting only 18% of the wild type FIV 3′ LTR sequence (Examples 15C and 15F).

Within other aspects of the invention, FIV vectors are provided with built-in nucleic acid sequences to help accommodate regulation of the transgene (e.g., therapeutic molecule, molecule that has adverse effects on FIV particle production as well as patient) in the vector through transcriptional or translational control.

### CONSTRUCTION OF PACKAGING EXPRESSION CASSETWS

As noted above, the present invention provides a variety of packaging expression cassettes which, in combination with the env expression cassettes of the present invention, enable the construction of packaging cell lines. Further introduction of FIV vector constructs into packaging cell lines enables the production of producer cell lines. The term “packaging expression cassettes” is used for expression constructs encoding gag/pol sequences alone, gag/pol and one or more of rev, vif or ORF 2 encoding sequences, or for constructs encoding for one or more of rev, vif or ORF 2 encoding sequences alone. These packaging expression cassettes may contain at least one RNA export element.

Representative examples of suitable packaging expression cassettes include gag/pol expression cassettes, which comprise a promoter and a sequence encoding gag/pol. Within another embodiment, the gag/pol expression cassette comprises a promoter, a sequence encoding gag/pol and at least one of rev, ORF 2 or vif wherein the promoter is operably linked to gag/pol and rev, vif or ORF 2.

Within other aspects of the invention, the expression cassette comprises a promoter and a sequence encoding either gag or pol alone.

Within further embodiments, rev expression cassettes are provided comprising a promoter and a sequence encoding rev. Within another embodiment, the rev expression cassette comprises a promoter, a sequence encoding rev and at least one of ORF 2 or vif, wherein the promoter is operably linked to rev and ORF 2 or vif.

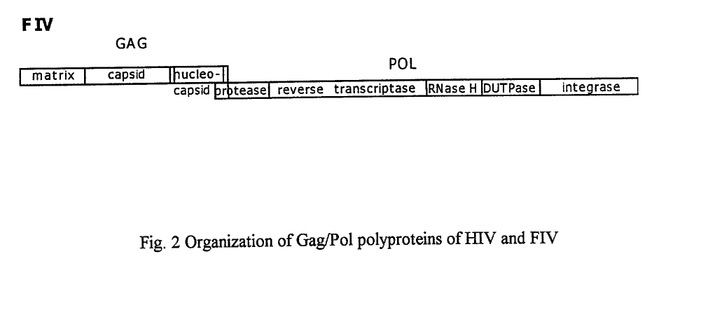

A demonstration of a packaging expression cassette encoding for gag/pol, rev, vif and ORF 2 is described in Example 5B and a detailed drawing describing the components of the gag/pol polyprotein are shown in FIG. 2. Briefly, FWV-derived gag/pol genes contain a gag region which encodes a variety of structural proteins that make up the core matrix, capsid and nucleocapsid, and a pol region which contains genes which encode (1) a protease for the processing of gag/pol and env proteins, (2) a reverse transcriptase polymerase, (3) an RNase H, (4) the enzyme deoxyuridine triphosphatase (dUTPase) and (5) an integrase, which is necessary for integration of the FIV provector into the host genome. Vif is a protein encoded by ORF 1 of FIV and believed to be the feline equivalent of the HIV viral infectivity factor, vif Orf 2 of FIV corresponds roughly in size and location to Orf S of Visna Virus S which encodes a protein capable of some degree of transactivation (Davis et al., PNAS USA 86:414-418, 1989). Although FIV-derived gag/pol, rev, vif and/or ORF 2 genes may be utilized to construct the gag/pol expression cassettes of the present invention, a variety of other non-retroviral (and non-viral) genes may also be utilized to construct the gag/pol expression cassette. For example, a gene which encodes retroviral RNase H may be replaced with genes which encode bacterial (e.g., E. coli or Thermus thermophilus) RNase H. Similarly, the FIV integrase gene may be replaced by other genes with similar function (e.g., yeast retrotransposon TY3 integrase).

Within one embodiment of the invention, the gag/pol expression cassette contains a heterologous promoter, and/or heterologous polyadenylation sequence. As utilized herein, “heterologous” promoters or polyadenylation sequences refers to promoters or polyadenylation sequences which are from a different source from which the gag/pol gene (and preferably the env gene and FIV vector construct) is derived from. Representative examples of suitable promoters include the Cytomegalovirus Immediate Early (“CMV IE”) promoter, the Herpes Simplex Virus Thynidine Kinase (“HSVTK”) promoter, the Rous Sarcoma Virus (“RSV”) promoter, the Adenovirus major-late promoter and the SV 40 promoter. Representative examples of suitable polyadenylation signals include the SV 40 late polyadenylation signal, the SV40 early polyadenylation signal and the bovine growth hormone polyadenylation/termination signal.

Within one embodiment of the invention, the gag/pol expression cassette includes a partial sequence of the gag/pol region, in other embodiments the expression cassette includes a full or partial sequence encoding the enzyme dUTPase. These embodiments may also include sequences encoding polypeptides of the gag/pol region, or variants thereof which are degenerate because of the genetic code or which contain mutations, or deletions that encode functional elements of the gag/pol region including dUTPase.

Within another embodiment of the invention, one or more packaging and rev-expression constructs can be expressed from an inducible promoter system (e.g., the tet-inducible promoter system described by Bujard et al., PNAS 89, 5547-5551, 1992). Alternatively, one or more packaging and rev-expression constructs are regulated by a translational control such as e.g. the use of the aptamer technology (Example 21)

Within preferred aspects of the present invention, gaglpol expression cassettes such as those described above will not co-encapsidate along with a replication competent virus.

### CONSTRUCTION OF ENVELOPE (ENV) EXPRESSION CASSETWS

Within other aspects of the present invention, env expression cassettes are provided which, in combination with the packaging expression cassettes and FIV vector constructs described above, enable the production of FIV vector particles and preclude formation of replication competent virus by homologous recombination. In addition, FIV viral particles described in this invention confer a particular specificity of the resultant vector particle (e.g., amphotropic, ecotropic, xenotropic, polytropic or pantropic). A representative example of an env expression cassette is shown in FIG. 3 C. Briefly, in a wild-type FIV the env gene encodes two principal proteins, the surface glycoprotein “SU′ and the transmembrane protein “TM”, which are translated as a polyprotein, and subsequently separated by proteolytic cleavage. Representative examples of the SU and TM proteins are the gp120 protein and gp41 protein in HIV, and the gp70 protein and p15e protein in MoMLV. In some retroviruses, a third protein designated the “R” peptide” of undetermined function, is also expressed from the env gene and separated from the polyprotein by proteolytic cleavage.

The term “env expression cassettes” is used for expression constructs encoding env sequences alone or env and one or more of rev, vif or ORF 2 encoding sequences.

A wide variety of env expression cassettes may be constructed given the disclosure provided herein, and utilized within the present invention to produce FIV vector particles. Within one aspect of the present invention, env expression cassettes are provided comprising a promoter operably linked to an env gene, wherein preferably no more than 6, 8, 10, 15, or 20 consecutive retroviral nucleotides are included upstream (5′) of and/or contiguous with said env gene. Within other aspects of the invention, env expression cassettes are provided comprising a promoter operably linked to an env gene, wherein the env expression cassette does not contain a consecutive sequence of greater than 20, preferably less than 15, more preferably less than 10, and most preferably less than 8 or 6 consecutive nucleotides which are found in a packaging or rev-expression cassette, and in particular, in a gag/pol expression cassette that will be utilized along with the env expression cassette to create a packaging cell line.

Within another aspect of the present invention, env expression cassettes are provided which contain a heterologous promoter, a heterologous leader sequence and/or heterologous polyadenylation sequence. As utilized herein, “heterologous” promoters, leaders or polyadenylation sequences refers to sequences which are from a different source from which the env gene (and preferably the packaging expression constructs and FIV vector construct) is derived from. Representative examples of suitable promoters include the CMV IE promoter, the HSVTK promoter, the RSV promoter, the Adenovirus major-late promoter and the SV 40 promoters. Representative examples of suitable polyadenylation signals include the SV 40 late polyadenylation signal, the SV40 early polyadenylation signal, and the bovine growth hormone termination/polyadenylation sequence. Preferably any such termination/polyadenylation sequence will not have any 10 bp stretch which has more than 80% homology to an FIV vector construct.

As described in more detail below in Examples 6, 7 and 20, FIV can be pseudotyped with the VSV-G envelope and the amphotropic MLV protein. The source of the viral env sequence can be derived from a wide variety of other retroviruses. For example envelope encoding sequences from VSV (Vesicular Stomatitis Virus); amphotropic, ecotropic, polytropic or xenotropic MLV; HIV, FIV, FeLV, SNV, alpha virus or GaLV (Gibbon Ape Leukemia Virus) may similarly be utilized.

Within one embodiment of the invention, modified forms of env expression cassettes are provided. For example truncated HIV envelopes or hybrid envelopes may be suitable for the production of FIV vector particles. Hybrid envelopes are understood to be env expression cassettes encoding viral envelopes plus heterologous viral or non-viral sequences that are added in addition or in place of viral env encoding sequences. Further, the env expression cassette may target the viral particle to a receptor of a particular cell type by linking the env coding sequences to an antibody or a particular ligand.

Within one embodiment of the invention, env expression cassettes are provided comprising a promoter and a sequence encoding a viral envelope sequence env alone. Within another embodiment of the invention, any of the above mentioned env expression cassettes are provided comprising a promoter, a sequence coding for env and at least one of rev, ORF 2 or vif wherein the promoter is operably linked to env and rev, ORF 2 or vif.

Within another embodiment of this invention, any of the above described env expression cassettes can be expressed from an inducible promoter system (e.g., the tet-inducible promoter system described by Bujard et al., PNAS 89, 5547-5551, 1992). Alternatively, one or envelope constructs are regulated by a translational control such as, e.g., the use of the aptamer technology (Example 21).

### PACKAGING/PRODUCER CELL LINES

Packaging cell lines suitable for use with the above described recombinant FIV vector constructs may be readily prepared given the disclosure provided herein. Within certain preferred embodiments, the packaging/producer cell lines are utilized to produce FIV vector particles that are substantially free from wild-type FIV vector particles. As utilized herein, it should be understood that FIV vector particles are “substantially free” from wild-type FIV vector particles if p24 is not detected in any statistically significant manner when presently available assays for p24 (e.g., IDEXX) are utilized.

Briefly, the parent cell line from which the packaging cell line is derived can be selected from a wide variety of mammalian cell lines, including for example, human cells, monkey cells, feline cells, dog cells, mouse cells, and the like.

Within one embodiment of the invention, potential packaging cell line candidates are screened by isolating the human placental alkaline phosphatase (PLAP) gene from the N2-derived retroviral vector pBAAP, and inserting the gene into the FIV vector construct (see FIG. 3). To generate infectious virus, the construct is co-transfected with a VSV-G encoding expression cassette (e.g., pMLP-G as described by Emi et al., J. Virology 65, 1202-1207, 1991; or pCMV-G, see U.S. Pat. No. 5,670,354) into 293T cells, and the virus harvested 48 hours after transfection. The resulting virus can be utilized to infect candidate host cells, which are subsequently FACS-analyzed using antibodies specific for PLAP. Candidate host cells include, e.g., human cells such as HeLa (ATCC CCL 2.1), HT-1080 (ATCC CCL 121), 293 (ATCC CRL 1573), Jurkat (ATCC TIB 153), supT1 (NIH AIDS Research and Reference reagent program catalog #100), and CEM (ATCC CCL 119) or feline cells such as CrFK (ATCC CCL 94), G355-5 (Ellen et al., Virology 187:165-177, 1992), MYA-1 (Dahl et al., J. Virology 61:1602-1608, 1987) or 3201-B (Ellen et al., Virology 187:165-177, 1992). In addition to analysis of cell surface PLAP expression, production of p24 and reverse transcriptase can also be analyzed in the assessment of suitable packaging cell lines.

After selection of a suitable host cell for the generation of a packaging cell line, one or more expression cassettes are introduced into the cell line in order to complement or supply in trans components of the FIV vector which have been deleted (see generally U.S. Ser. No. 08/240,030, filed May 9, 1994; see also U.S. Ser. No. 07/800,921, filed Nov. 27, 1991).

Representative examples of suitable expression cassettes include packaging expression cassettes, rev-expression cassettes and envelope expression cassettes, which are described in more detail below. Briefly, packaging expression cassettes encode either gag/pol sequences alone, gag/pol sequences and one or more of vitf rev or ORF 2 or expression cassettes encoding one or more of vif, rev or ORF 2 alone and may contain at least one RNA export element. Envelope expression cassettes encode either an env sequence alone or env and one or more of vif, rev or ORF 2.

Utilizing the above-described expression cassettes, a wide variety of packaging cell lines can be generated. Any combination of the above mentioned expression cassettes can be used for the production of FIV-derived packaging cell lines. For example, within one aspect packaging cell lines are provided comprising an envelope expression cassette and an expression cassette that comprises a sequence encoding gag/pol, and at least one RNA export element, wherein the promoter is operably linked to the sequence encoding gag/pol.

Within other aspects, packaging cell lines are provided comprising a promoter and a sequence encoding ORF 2, vif, rev, or an envelope (e.g., VSV-G), wherein the promoter is operably linked to the sequence encoding ORF 2, vi rev, or, the envelopeWithin further embodiments, the packaging cell line may further comprise a sequence encoding any one or more of rev, ORF 2 or vif. For example, the packaging cell line may contain only ORF 2, vif or rev alone, ORF 2 and vif, ORF 2 and rev, vif and rev or all three of ORF 2, vif and rev.

Within further aspects of the invention, for packaging cell lines containing inducible expression cassettes cited above, additional expression cassettes facilitating the activation/regulation of the inducible promoter or other regulatable components may be incorporated.

Within other aspects, the packaging cell line is derived from a feline or human parent cell, contains FIV-derived packaging constructs coding for a dUT~ase, and FIV, amphotropic MLV or VSV-G derived env expression cassettes for the use to deliver nucleic acid sequences to cats. Within yet other aspects, packaging cell lines are provided which comprise an env expression cassette derived from any virus, or, hybrid env cassette. For such hybrid env cassettes, the coding region may be derived from more than one viral envelope.

Within another embodiment, the expression cassette is stably integrated. Within yet another embodiment, the packaging cell line, upon introduction of an FIV vector, produces particles at a concentration of greater than 10³, 10⁴, 10⁵, 10⁶, 10⁷, 10⁸, or, 10⁹ cfu/nml. Within further embodiments the promoter is inducible. Within certain preferred embodiments of the invention, the packaging cell line, upon introduction of an FIV vector, produces particles that are free of replication competent virus. Within further embodiments, expression of a heterologous sequence from the FIV vector may be under transcriptional or translational control.

### GENES OF INTEREST/HETEROLOGOUS NUCLEIC ACID MOLECULES

A wide variety of nucleic acid molecules may be carried and/or expressed by the FIV vector particles of the present invention. As used herein, “pathogenic agent” refers to a cell that is responsible for a disease state. Representative examples of pathogenic agents include tumor cells, autoreactive immune cells, hormone secreting cells, cells which lack a function that they would normally have, cells that have an additional inappropriate gene expression which does not normally occur in that cell type, and cells infected with bacteria, viruses, or other intracellular parasites. In addition, as used herein “pathogenic agent” may also refer to a cell that has become tumorigenic due to inappropriate insertion of nucleic acid molecules contained by the FIV vector into a host cell's genome.

Examples of nucleic acid molecules which may be carried and/or expressed by FIV vector particles of the present invention include genes and other nucleic acid molecules which encode a substance, as well as biologically active nucleic acid molecules such as inactivating sequences that incorporate into a specified intracellular nucleic acid molecule and inactivate that molecule. A nucleic acid molecule is considered to be biologically active when the molecule itself provides the desired benefit. For example, the biologically active nucleic acid molecule may be an inactivating sequence that incorporates into a specified intracellular nucleic acid molecule and inactivates that molecule, or the molecule may be a tRNA, rRNA or mRNA that has a configuration that provides a binding capability.

Substances which may be encoded by the nucleic acid molecules described herein include proteins (e.g., antibodies including single chain molecules), immunostimulatory molecules (such as antigens) immunosuppressive molecules, blocking agents, palliatives (such as toxins, antisense ribonucleic acids, ribozymes, enzymes, and other material capable of inhibiting a function of a pathogenic agent) cytokines, various polypeptides or peptide hormones, their agonists or antagonists, where these hormones can be derived from tissues such as the pituitary, hypothalamus, kidney, endothelial cells, liver, pancreas, bone, hemopoetic marrow, and adrenal. Such polypeptides can be used for induction of growth, regression of tissue, suppression of immune responses, apoptosis, gene expression, blocking receptor-ligand interaction, immune responses and can be treatment for certain anemias, diabetes, infections, high blood pressure, abnormal blood chemistry or chemistries (e.g., elevated blood cholesterol, deficiency of blood clotting factors, elevated LDL with lowered HDL), levels of Alzheimer associated amyloid protein, bone erosion/calcium deposition, and controlling levels of various metabolites such as steroid hormones, purines, and pyrimidines.

For palliatives, when “capable of inhibiting a function” is utilized within the context of the present invention, it should be understood that the palliative either directly inhibits the function or indirectly does so, for example, by converting an agent present in the cells from one which would not normally inhibit a function of the pathogenic agent to one which does. Examples of such functions for viral diseases include adsorption, replication, gene expression, assembly, and exit of the virus from infected cells. Examples of such functions for cancerous diseases include cell replication, susceptibility to external signals (e.g., contact inhibition), and lack of production of anti-oncogene proteins. Examples of such functions for cardiovascular disease include inappropriate growth or accumulation of material in blood vessels, high blood pressure, undesirable blood levels of factors such as cholesterol or low density lipoprotein that predispose to disease, localized hypoxia, and inappropriately high and tissue-damaging levels of free radicals. Examples of such functions for neurological conditions include pain, lack of dopamine production, inability to replace damaged cells, deficiencies in motor control of physical activity, inappropriately low levels of various peptide hormones derived from neurological tissue such as the pituitary or hypothalamus, accumulation of Alzheimer's Disease associated amyloid plaque protein, and inability to regenerate damaged nerve junctions. Examples of such functions for autoimmune or inflammatory disease include inappropriate production of cytokines and lymphokines, inappropriate production and existence of autoimmune antibodies and cellular immune responses, inappropriate disruption of tissues by proteases and collagenases, lack of production of factors normally supplied by destroyed cells, and excessive or aberrant regrowth of tissues under autoimmune attack.

Within one aspect of the present invention, methods are provided for administration of a recombinant FIV vector which directs the expression of a palliative. Representative examples of palliatives that act directly to inhibit the growth of cells include toxins such as ricin (Lamb et al., Eur. J. Biochem. 148:265-270, 1985), abrin (Wood et al., Eur. J. Biochem. 198:723-732, 1991; Evensen et al., J. of Biol. Chem. 266:6848-6852, 1991; Collins et al., J. of Biol Chem. 265:8665-8669, 1990; Chen et al., Fed. of Eur. Biochem Soc. 309:115-118, 1992), diphtheria toxin (Tweten et al., J. Biol. Chem. 260:10392-10394, 1985), cholera toxin (Mekalanos et al., Nature 306:551-557, 1983; Sanchez & Holmgren, PNAS 86:481-485, 1989), gelonin (Stirpe et al., J. Biol. Chem. 255:6947-6953, 1980), pokeweed (Irvin, Pharmac. Ther. 21:371-387, 1983), antiviral protein (Barbieri et al., Biochem. J. 203:55-59, 1982; Irvin et al., Arch. Biochem. & Biophys. 200:418-425, 1980; Irvin, Arch. Biochem. &Biophys. 169:522-528, 1975), tritin, Shigella toxin (Calderwood et al., PNAS 84:4364-4368, 1987; Jackson et al., Microb. Path. 2:147-153, 1987), and Pseudomonas exotoxin A (Carroll and Collier, J. Biol. Chem. 262:8707-8711, 1987). A detailed description of recombinant retroviruses which express Russel's Viper Venom is provided in U.S. Ser. No. 08/368,574, filed Dec. 30, 1994.

Within other aspects of the invention, the FIV vector carries a gene specifying a product which is not in itself toxic, but when processed or modified by a protein, such as a protease specific to a viral or other pathogen, is converted into a toxic form. For example, recombinant retrovirus could carry a gene encoding a proprotein chain, which becomes toxic upon processing by the FIV protease. More specifically, a synthetic inactive proprotein form of the toxic ricin or diphtheria A chains could be cleaved to the active form by arranging for the FIV virally encoded protease to recognize and cleave off an appropriate “pro” element.

Within a related aspect of the present invention, FIV vectors are provided which direct the expression of a gene product(s) that activates a compound with little or no cytotoxicity into a toxic product. Briefly, a wide variety of gene products which either directly or indirectly activate a compound with little or no cytotoxicity into a toxic product may be utilized within the context of the present invention. Representative examples of such gene products include HSVTK and VZVTK which selectively monophosphorylate certain purine arabinosides and substituted pyrimidine compounds, converting them to cytotoxic or cytostatic metabolites. More specifically, exposure of the drugs ganciclovir, acyclovir, or any of their analogues (e.g., FIAC, DHPG) to HSVTK, phosphorylates the drug into its corresponding active nucleotide triphosphate form.

In a manner similar to the preceding embodiment, FIV vectors may be generated which carry a gene for phosphorylation, phosphoribosylation, ribosylation, or other metabolism of a purine- or pyrimidine-based drug. Such genes may have no equivalent in mammalian cells, and might come from organisms such as a virus, bacterium, fungus, or protozoan. Representative examples include: E. coli guanine phosphoribosyl transferase (“gpt”) gene product, which converts thioxanthine into thioxanthine monophosphate (see Besnard et al., Mol. Cell. Biol. 7:4139-4141, 1987); alkaline phosphatase, which will convert inactive phosphorylated compounds such as mitomycin phosphate and doxorubicin-phosphate to toxic dephosphorylated compounds; fungal (e.g., Fusarium oxysporum) or bacterial cytosine deaminase which will convert 5-fluorocytosine to the toxic compound 5-fluorouracil (Mullen, PNAS 89:33, 1992); carboxypeptidase G2 which will cleave the glutamic acid from para-N-bis (2-chloroethyl) aminobenzoyl glutamic acid, thereby creating a toxic benzoic acid mustard; and Penicillin-V amidase, which will convert phenoxyacetabide derivatives of doxorubicin and melphalan to toxic compounds. Conditionally lethal gene products of this type have application to many presently known purine- or pyrimidine-based anticancer drugs, which often require intracellular ribosylation or phosphorylation in order to become effective cytotoxic agents. The conditionally lethal gene product could also metabolize a nontoxic drug, which is not a purine or pyrimidine analogue, to a cytotoxic form (see Searle et al., Brit. J. Cancer 53:377-384, 1986).

Additionally, in the instance where the target pathogen is a mammalian virus, FIV vectors may be constructed to take advantage of the fact that mammalian viruses in general tend to have “immediate early” genes, which are necessary for subsequent transcriptional activation of other viral promoter elements. Gene products of this nature are excellent candidates for intracellular signals (or “identifying agents”) of viral infection. Thus, conditionally lethal genes transcribed from transcriptional promoter elements that are responsive to such viral “immediate early” gene products could specifically kill cells infected with any particular virus. Additionally, since the human and interferon promoter elements are transcriptionally activated in response to infection by a wide variety of nonrelated viruses, the introduction of vectors expressing a conditionally lethal gene product like HSVTK, for example, from these viral-responsive elements (VREs) could result in the destruction of cells infected with a variety of different viruses.

In another embodiment of the invention, FIV vectors are provided that produce substances such as inhibitor palliatives, that inhibit viral assembly. In this context, the recombinant retrovirus codes for defective gag, pot, env or other viral particle proteins or peptides, which inhibit in a dominant fashion the assembly of viral particles. Such inhibition occurs because the interaction of normal subunits of the viral particle is disturbed by interaction with the defective subunits.

One way of increasing the effectiveness of inhibitory palliatives is to express inhibitory genes, such as viral inhibitory genes, in conjunction with the expression of genes which increase the probability of infection of the resistant cell by the virus in question. The result is a nonproductive “dead-end” event, which would compete for productive infection events. In the specific case of FIV, a recombinant retrovirus may be administered that inhibits FIV replication (by expressing anti-sense tat, etc., as described above) and also overexpress proteins required for infection, such as CD4. In this way, a relatively small number of vector-infected FIV-resistant cells act as a “sink” or “magnet” for multiple nonproductive fuision events with free virus or virally infected cells.

In another embodiment of the invention, FIV vectors are provided for the expression substances such as inhibiting peptides or proteins specific for viral protease. Viral protease cleaves the viral gag and gag/pol proteins into a number of smaller peptides. Failure of this cleavage in all cases leads to complete inhibition of production of infectious retroviral particles. The HIV protease is known to be an aspartyl protease, and these are known to be inhibited by peptides made from amino acids from protein or analogues. FIV vectors that inhibit HIV will express one or multiple fused copies of such peptide inhibitors.

Administration of the FIV vectors discussed above should be effective against many virally linked diseases, cancers, or other pathogenic agents.

In yet another aspect, FIV vectors are provided which have a therapeutic effect by encoding one or more ribozymes (RNA enzymes) (Haseloff and Gerlach, Nature 334:585, 1989) which will cleave, and hence inactivate, RNA molecules corresponding to a pathogenic function. Since ribozymes function by recognizing a specific sequence in the target RNA and this sequence is normally 12 to 17 bp, this allows specific recognition of a particular RNA sequence corresponding to a pathogenic state, such as HIV tat, and toxicity is specific to such pathogenic state. Representative examples of suitable ribozymes include hammerhead ribozymes (see Rossi et al., Pharmac. Ther 50:245-254, 1991) and hairpin ribozymes (Hämpel et al., Nucl. Acids Res. 18:299-304, 1990; U.S. Pat. No. 5,254,678) and Tetrahymena based ribozymes (U.S. Pat. No. 4,987,071). Additional specificity may be achieved in some cases by making this a conditional toxic palliative, as discussed above.

In still another aspect, FIV vectors are provided comprising a biologically active nucleic acid molecule that is an antisense sequence (an antisense sequence may also be encoded by a nucleic acid sequence and then produced within a host cell via transcription). Briefly, antisense sequences are designed to bind to RNA transcripts, and thereby prevent cellular synthesis of a particular protein, or prevent use of that RNA sequence by the cell. Representative examples of such sequences include antisense thymidine kinase, antisense dihydrofolate reductase (Maher and Dolnick, Arch. Biochem. &Biophys. 253:214-220, 1987; Bzik et al., PNAS 84:8360-8364, 1987), antisense BER2 (Coussens et al., Science 230:1132-1139, 1985), antisense ABL (Fainstein et al., Oncogene 4:1477-1481, 1989), antisense Myc (Stanton etal., Nature 310:423-425, 1984) and antisense ras, as well as antisense sequences which block any of the enzymes in the nucleotide biosynthetic pathway. In other embodiments, the antisense sequence is selected from the group consisting of sequences which encode influenza virus, HIV, HSV, HPV, CMV, and HBV. The antisense sequence may also be an antisense RNA complementary to RNA sequences necessary for pathogenicity. Alternatively, the biologically active nucleic acid molecule may be a sense RNA (or DNA) complementary to RNA sequences necessary for pathogenicity.

Within a further embodiment of the invention antisense RNA may be utilized as an anti-tumor agent in order to induce a potent Class I restricted response. Briefly, in addition to binding RNA and thereby preventing translation of a specific mRNA, high levels of specific antisense sequences are believed to induce the increased expression of interferons (including gamma-interferon), due to the formation of large quantities of double-stranded RNA. The increased expression of gamma interferon, in turn, boosts the expression of MHC Class I antigens. Preferred antisense sequences for use in this regard include actin RNA, myosin RNA, and histone RNA Antisense RNA which forms a mismatch with actin RNA is particularly preferred.

In another embodiment, FIV vectors of the invention express a surface protein that is itself therapeutically beneficial. For example, in the particular case of HIV, expression of the human CD4 protein specifically in HIV-infected cells may be beneficial in two ways:

1. Binding of CD4 to HIV env intracellularly could inhibit the formation of viable viral particles much as soluble CD4 has been shown to do for free virus, but without the problem of systematic clearance and possible immunogenicity, since the protein will remain membrane bound and is structurally identical to endogenous CD4 (to which the patient should be immunologically tolerant).

2. Since the CD4/HIV env complex has been implicated as a cause of cell death, additional expression of CD4 (in the presence of excess HIV-env present in HIV-infected cells) leads to more rapid cell death and thus inhibits viral dissemination. This may be particularly applicable to monocytes and macrophages, which act as a reservoir for virus production as a result of their relative refractility to HIV-induced cytotoxicity (which, in turn, is apparently due to the relative lack of CD4 on their cell surfaces).

Still further aspects of the present invention relate to FIV vectors capable of immunostimulation. Briefly, the ability to recognize and defend against foreign pathogens is essential to the function of the immune system. In particular, the immune system should be capable of distinguishing “self” from “nonself” (ie., foreign), so that the defensive mechanisms of the host are directed toward invading entities instead of against host tissues. Cytolytic T lymphocytes (CTLs) are typically induced, or timulated, by the display of a cell surface recognition structure, such as a processed, athogen-specific peptide, in conjunction with a MIC class I or class II cell surface rotein.

Diseases suitable to treatment include viral infections such as influenza virus, respiratory syncytial virus, HPV, HBV, HCV, EBV, HIV, HSV, FeLV, FIV, Hantavirus, HTLV I, HTLV II and CMV, cancers such as melanomas, renal carcinoma, breast cancer, ovarian cancer and other cancers, and heart disease.

In one embodiment, the invention provides methods for stimulating a specific immune response and/or inhibiting viral spread by using FIV vectors that direct the expression of an antigen or modified form thereof in susceptible target cells, wherein the antigen is capable of either (1) initiating an immune response to the viral antigen or (2) preventing the viral spread by occupying cellular receptors required for viral interactions. Expression of the protein may be transient or stable with time. Where an immune response is to be stimulated to a pathogenic antigen, the FIV vector is preferably designed to express a modified form of the antigen which will stimulate an immune response and which has reduced pathogenicity relative to the native antigen. This immune response is achieved when cells present antigens in the correct manner, i.e., in the context of the MHC class I and/or II molecules along with accessory molecules such as CD3, ICAM-1, ICAM-2, LFA-1, or analogs thereof (e.g., Altmann et al., Nature 338:512, 1989).

An immune response can also be achieved by transferring to an appropriate immune cell (such as a T lymphocyte) (a) the gene for the specific T-cell receptor that recognizes the antigen of interest (in the context of an appropriate NMC molecule if necessary), (b) the gene for an immunoglobulin which recognizes the antigen of interest, or (c) the gene for a hybrid of the two which provides a CTL response in the absence of the MHC context. Thus, recombinant retroviruses may also be used as an immunostimulant, immunomodulator, or vaccine, etc.

In the particular case of disease caused by mV infection, where immunostimulation is desired, the antigen generated from a recombinant retrovirus may be in a form which will elicit either or both an HLA class I- or class II-restricted immune response. In the case of HIV envelope antigen, for example, the antigen is preferably selected from gp 160, gp 120, and gp 41, which have been modified to reduce their pathogenicity. In particular, the selected antigen is modified to reduce the possibility of syncytia, to avoid expression of epitopes leading to a disease enhancing immune response, to remove immunodominant, but haplotype-specific epitopes or to present several haplotype-specific epitopes, and allow a response capable of eliminating cells infected with most or all strains of HV. The haplotype-specific epitopes can be further selected to promote the stimulation of an immune response within an animal, which is cross-reactive against other strains of HlIV. Antigens from other HIV genes or combinations of genes, such as gag, pol, rev, vif, net prot, gag/pol, gag prot, etc., may also provide protection in particular cases.

HIV is only one example. This approach may be utilized for many virally linked diseases or cancers where a characteristic antigen (which does not need to be a membrane protein) is expressed. Representative examples of such “disease-associated” antigens all or portions of various eukaryotic (including for example, parasites), prokaryotic (e.g., bacterial) or viral pathogens. Representative examples of viral pathogens include the Hepatitis B Virus (“HBV”) and Hepatitis C Virus (“HCV”; see U.S. Ser. No. 08/102/132), Human Papiloma Virus (“HPV”; see WO 92/05248; WO 90/10459; EPO 133,123), Epstein-Barr Virus (“EBV”; see EPO 173,254; JP 1,128,788; and U.S. Pat. Nos. 4,939,088 and 5,173,414), Feline Leukemia Virus (“FeLV”; see U.S. Ser. No. 07/948,358; EPO 377,842; WO 90108832; WO 93/09238), Feline Immunodeficiency Virus (“FIV”; U.S. Pat. No. 5,037,753; WO 92/15684; WO 90/13573; and JP 4,126,085), HTLV I and II, and Human Immunodeficiency Virus (“HIV”; see U.S. Ser. No. 07/965,084).

In accordance with the immunostimulation aspects of the invention, substances which are carried and/or expressed by the FIV vectors of the present invention may also include “immunomodulatory factors,” many of which are set forth above. Immunomodulatory factors refer to factors that, when manufactured by one or more of the cells involved in an immune response, or, which when added exogenously to the cells, causes the immune response to be different in quality or potency from that which would have occurred in the absence of the factor. The factor may also be expressed from a non-recombinant retrovirus derived gene, but the expression is driven or controlled by the recombinant retrovirus. The quality or potency of a response may be measured by a variety of assays known to one of skill in the art including, for example, in vitro assays which measure cellular proliferation (e.g., ³H thymidine uptake), and in vitro cytotoxic assays (e.g., which measure ⁵¹Cr release) (see, Warner et al., AIDS Res. and Human Retroviruses 7:645-655, 1991). Immunomodulatory factors may be active both in vivo and ex vivo.

Representative examples of such factors include cytokines, such as IL-1, IL-2 (Karupiah et al., J. Immunology 144:290-298, 1990; Weber et al., J. Exp. Med. 166:1716-1733, 1987; Gansbacher etal., J. Exp. Med 172:1217-1224, 1990; U.S. Patent No. 4,738,927), IL-3, IL-4 (Tepper et al., Cell 57:503-512, 1989; Columbek et al., Science 254:713-716, 1991; U.S. Pat. No. 5,017,691), IL-5, IL-6 (Brakenhof et al., J. Immunol. 139:4116-4121, 1987; WO 90/06370), IL-7 (U.S. Pat. No. 4,965,195), IL-8, IL-9, IL-10, IL-11, IL-12, IL-13 (Cytokine Bulletin, Summer 1994), IL-14 and IL-15, particularly IL-2, IL-4, IL-6, IL-12, and IL-13, alpha interferon (Finter et al., Drugs 42(5):749-765, 1991; U.S. Pat. No. 4,892,743; U.S. Pat. No.4,966,843; WO 85/02862; Nagata et al., Nature 284:316-320, 1980; Farilletti et al., Methods in Enz. 78:387-394, 1981; Twu et al., PNAS 86:2046-2050, 1989; Faktor et al., Oncogene 5:867-872, 1990), beta interferon (Seif et al., J. Virol 65:664-671, 1991), gamma interferons (Radford et al., The American Society of Hepatology 2008-2015, 1991; Watanabe et al., PNAS 86:9456-9460, 1989; Gansbacher et al., Cancer Research 50:7820-7825, 1990; Maio et al., Can. Immunol. Immunother. 30:34-42, 1989; U.S. Pat. No. 4,762,791; U.S. Pat. No. 4,727,138), G-CSF (U.S. Pat. Nos. 4,999,291 and 4,810,643), GM-CSF (WO 85/04188), tumor necrosis factors (TNFs) (Jayaraman et al., J. Immunology 144:942-951, 1990), CD3 (Krissanen et al., Immunogenetics 26:258-266, 1987), ICAM-1 (Altman et al., Nature 338:512-514, 1989; Simmons et al., Nature 331:624-627, 1988), ICAM-2, LFA-1, LFA-3 (Wallner et al., X Exp. Med 166(4):923-932, 1987), MHC class I molecules, MHC class II molecules, B7.1-.3, b₂-microglobulin (Parnes et al., PNAS 78:2253-2257, 1981), chaperones such as calnexin, MHC linked transporter proteins or analogs thereof (Powis et al., Nature 354:528-531, 1991). Immunomodulatory factors may also be agonists, antagonists, or ligands for these molecules. For example soluble forms of receptors can often behave as antagonists for these types of factors, as can mutated forms of the factors themselves.

The choice of which immunomodulatory factor to include within a FIV vector may be based upon known therapeutic effects of the factor, or, experimentally determined. For example, a known therapeutic effector in chronic hepatitis B infections is alpha interferon. This has been found to be efficacious in compensating a patient's immunological deficit, and thereby assisting recovery from the disease. Alternatively, a suitable immunomodulatory factor may be experimentally determined. Briefly, blood samples are first taken from patients with a hepatic disease. Peripheral blood lymphocytes (PBLs) are restimulated in vitro with autologous or HLA matched cells (e.g., EBV transformed cells) that have been transduced with a recombinant retrovirus which directs the expression of an immunogenic portion of a hepatitis antigen and the immunomodulatory factor. These stimulated PBLs are then used as effectors in a CTL assay with the HLA matched transduced cells as targets. An increase in CTL response over that seen in the same assay performed using HLA matched stimulator and target cells transduced with a vector encoding the antigen alone, indicates a useful immunomodulatory factor. Within one embodiment of the invention, the immunomodulatory factor gamma interferon is particularly preferred.

The present invention also includes FIV vectors which encode immunogenic portions of desired antigens including, for example, viral, bacterial or parasite antigens. For example, at least one immunogenic portion of a hepatitis B antigen can be incorporated into an FIV vector. The immunogenic portion(s) which are incorporated into the FIV vector may be of varying length, although it is generally preferred that the portions be at least 9 amino acids long, and may include the entire antigen. Immunogenicity of a particular sequence is often difficult to predict, although T cell epitopes may be predicted utilizing the HLA A2.1 motif described by Falk et al. (Nature 351:290, 1991). From this analysis, peptides may be synthesized and used as targets in an in vitro cytotoxic assay. Other assays, however, may also be utilized, including, for example, ELISA which detects the presence of antibodies against the newly introduced vector, as well as assays which test for T helper cells, such as gamma-interferon assays, IL-2 production assays, and proliferation assays.

Within one embodiment of the present invention, at least one immunogenic portion of a hepatitis C antigen can be incorporated into an FIV vector. Preferred immunogenic portion(s) of hepatitis C may be found in the C and NS3-NS4 regions since these regions are the most conserved among various types of hepatitis C virus (Houghton et al., Hepatology 14:381-388, 1991). Particularly preferred immunogenic portions may be determined by a variety of methods. For example, as noted above for the hepatitis B virus, identification of immunogenic portions of the polypeptide may be predicted based upon amino acid sequence. Briefly, various computer programs which are known to those of ordinary skill in the art may be utilized to predict CTL epitopes. For example, CTL epitopes for the HLA A2.1 haplotype may be predicted utilizing the HLA A2.1 motif described by Falk et al. (Nature 351:290, 1991). From this analysis, peptides are synthesized and used as targets in an in vitro cytotoxic assay.

Other disease-associated antigens which may be carried by the gene delivery constructs of the present invention include, for example immunogenic, non-tumorigenic forms of altered cellular components which are normally associated with tumor cells (see U.S. Ser. No. 08/104,424). Representative examples of altered cellular components which are normally associated with tumor cells include ras* (wherein * is understood to refer to antigens which have been altered to be non-tumorigenic), p53 *, Rb*, altered protein encoded by Wilms' tumor gene, ubiquitin*, mucin, protein encoded by the DCC, APC, and MCC genes, as well as receptors or receptor-like structures such as neu, thyroid hormone receptor, Platelet Derived Growth Factor (“PDGF”) receptor, insulin receptor, Epidermal Growth Factor (“EGF”) receptor, and the Colony Stimulating Factor (“CSF”) receptor.

Immunogenic portions of the disease-associated antigens described herein may be selected by a variety of methods. For example, the HLA A2.1/K^{b} transgenic mouse has been shown to be usefuil as a model for human T-cell recognition of viral antigens. Briefly, in the influenza and hepatitis B viral systems, the murine T-cell receptor repertoire recognizes the same antigenic determinants recognized by human T-cells. In both systems, the CTL response generated in the HLA A2.1/K^{b} transgenic mouse is directed toward virtually the same epitope as those recognized by human CTLs of the HLA A2.1 haplotype (Vitiello et al., J. Ex. Med. 173:1007-1015, 1991; Vitiello et al., Abstract of Molecular Biology of Hepatitis B Virus Symposia, 1992).

Immunogenic proteins of the present invention may also be manipulated by a variety of methods known in the art, in order to render them more immunogenic. Representative examples of such methods include: adding amino acid sequences that correspond to T helper epitopes; promoting cellular uptake by adding hydrophobic residues; by forming particulate structures; or any combination of these (see generally, Hart, op. cit., Milich et al., PNAS 85:1610-1614, 1988; Willis, Nature 340:323-324, 1989; Griffiths et al., J. ViroL 65:450-456, 1991).

Sequences which encode the above-described nucleic acid molecules may be obtained from a variety of sources. For example, plasmids which contain sequences that encode altered cellular products may be obtained from a depository such as the American Type Culture Collection (ATCC, Rockville, Md.), or from commercial sources such as Advanced Biotechnologies (Columbia, Md.). Representative examples of plasmids containing some of the above-described sequences include ATCC No. 41000 (containing a G to T mutation in the 12th codon of ras), and ATCC No. 41049 (containing a G to A mutation in the 12th codon).

Other nucleic acid molecules that encode the above-described substances, as well as other nucleic acid molecules that are advantageous for use within the present invention, may be readily obtained from a variety of sources, including for example depositories such as the American Type Culture Collection (ATCC, Rockville, Maryland), or from commercial sources such as British Bio-Technology Limited (Cowley, Oxford England). Representative examples include BBG 12 (containing the GM-CSF gene coding for the mature protein of 127 amino acids), BBG 6 (which contains sequences encoding gamma interferon), ATCC No. 39656 (which contains sequences encoding TNF), ATCC No. 20663 (which contains sequences encoding alpha interferon), ATCC Nos. 31902, 31902 and 39517 (which contains sequences encoding beta interferon), ATCC No 67024 (which contains a sequence which encodes Interleukin-lb), ATCC Nos. 39405, 39452, 39516, 39626 and 39673 (which contains sequences encoding Interleukin-2), ATCC Nos. 59399, 59398, and 67326 (which contain sequences encoding Interleukin-3), ATCC No. 57592 (which contains sequences encoding Interleukin-4), ATCC Nos. 59394 and 59395 (which contain sequences encoding Interleukin-5), and ATCC No. 67153 (which contains sequences encoding Interleukin-6).

Molecularly cloned genomes which encode the hepatitis B virus may be obtained from a variety of sources including, for example, the American Type Culture Collection (ATCC, Rockville, Md.). For example, ATCC No. 45020 contains the total genomic DNA of hepatitis B (extracted from purified Dane particles) (see FIG. 3 of Blum et al., HYG 5(5):154-158, 1989) in the BamH I site of pBR322 (Moriarty et al., PNAS 78:2606-2610, 1981). (Note that correctable errors occur in the sequence of ATCC No. 45020.)

Alternatively, cDNA sequences for use with the present invention may be obtained from cells which express or contain the sequences. Briefly, within one embodiment mRNA from a cell which expresses the gene of interest is reverse transcribed with reverse transcriptase using oligo dT or random primers. The single stranded cDNA may then be amplified by PCR (see U.S. Pat. Nos. 4,683,202, 4,683,195 and 4,800,159). See also PCR Technology: Principles and Applications for DNA Amplification, Erlich (ed.), Stockton Press, 1989) utilizing oligonucleotide primers complementary to sequences on either side of desired sequences. In particular, a double stranded DNA is denatured by heating in the presence of heat stable Taq polymerase, sequence specific DNA primers, ATP, CTP, GTP and TTP. Double-stranded DNA is produced when synthesis is complete. This cycle may be repeated many times, resulting in a factorial amplification of the desired DNA.

Nucleic acid molecules which are carried and/or expressed by the FIV vectors described herein may also be synthesized, for example, on an Applied Biosystems Inc. DNA synthesizer (e.g., APB DNA synthesizer model 392 (Foster City, California).

### REGULATED EXPRESSION OF STRUCTURAL AND THERAPEUTIC GE PRODUCTS

As noted above, the present invention provides tightly controllable or regulated expression vectors (including env, rev, gag, pol and/or gag/pol expression cassettes, FIV vectors, and FIV vector particles) which, through transcriptional and/or translational control mechanisms, allow the production of highly toxic or fusogenic proteins or such molecules that need to be regulated in vivo. Such vectors, in contrast to other inducible promoter systems, do not exhibit pleiotropic effects on eukaryotic cells.

For example, in one aspect of the present invention expression vectors are provided wherein a LAP (Lac Activator Protein; Levine and Shenk, Molec. Cell. Biol. 10:3343-3356, 1990; PNAS 88:5072-5076, 1991) promoter system is operably linked to and capable of expressing a sequence encoding one of the aforementioned toxic or fbsogenic proteins. Briefly, LAPs are potent activators of several promoters (SV40, vaccinia virus promoter, T3 bacteriophage) containing lac operator sequences positioned either upstream or downstream of the transcription unit. A single lac operator allows for transactivation, multiple operators act synergistically. Binding affinity and specificity of lac repressor to operator sequences are very high with a binding affinity of 10⁻¹³ M. The LAP fusion protein has three components: (1) lac repressor; (2) SV40 nuclear localization signal; and (3) VP16 (transcription activation domain of HSV). One representative illustration of such a system is provided in Example 4 below.

Within another aspect of the present invention, expression vectors are provided which comprise a tet (tetracycline-controlled) promoter system which is operably linked to and capable of expressing one of the above-mentioned toxic or fusogenic proteins. Briefly, the tet promoter is negatively regulated by the presence of tetracycline. Within one embodiment, a constitutively expressed tetracycline-controlled transactivator (tTA) may be generated by fusing the tet repressor with the activating domain of VP16 from Herpes simplex (Gossen and Bujard, PNAS 89:5547-5551, 1992). In the absence of tetracycline, this transactivator stimulates transcription from a minimal CMV-derived promoter sequence which also contains tet operator sequences located upstream of the promoter. Integration of a sequence encoding a fusogenic protein such as VSV-G downstream from the tTA-dependent promoter leads to high level expression of VSV-G in the absence of tetracycline. Such vectors are particularly useful for the production of recombinant FIV proteins, since tetracycline may be maintained in the medium while packaging, and subsequently, producer cell lines are made. After a sufficient number of producer cell lines are grown up, tetracycline may then be removed from the medium, thus allowing the production of VSV-G containing FIV vector particles.

Other examples of tightly controllable promoter or promoter systems which may be utilized within the present invention include GAL1 (J. Biotech. 32(2):179-89, 1994); Gene, 156(1):19-25, 1995; P.N.A.S. 89(23):11589-93, 1992); steroids such as Dexamethasone (Biochim Biophys Acta, 219(3):653-9, 1994; Nawa et al., J. Biol. Chem. 261(36):16883-16888, 1986; J. Vir., 64(10):5132-42. 1990); heavy metal regulated systems such as Metallothionein (J. Vir., 68(9):5602-12, 1994; Carc., 14(8): 1643-9, 1993; J. Vir. Methods, 33(1-2): 13547, 1993); T7/lac (J. Biol Chem, 269(16):12106-10, 1994; Biochem Biophys Res Comm., 188(3):972-81, 1992); Tat and or tat/rev inducible lentiviral promoter systems, e.g., mV-1 or 2 (see e.g., Leukemia, 7Suppl 2:S61-5, 1993; P.N.A.S. 89(1):182-6, 1992); Heat shock (J. Biol Chem, 268(9):6708-13, 1993); NF-KB responsive (Mol Aspects Med, 14(3):171-90, 1993); Cyclic AMP (Roesler et al., J. Biol. Chem. 263(19):9063-9066, 1988).

As noted above, within other aspects of the present invention expression vectors are provided which are tightly regulated through translational mechanisms. For example, within one aspect of the present invention expression vectors are provided comprising a promoter (e.g., one of the promoters discussed above) operably linked to a nucleic acid molecule encoding a toxic or fusogenic protein, and a selenium responsive element, wherein one or more cysteine residues in the nucleic acid molecule have been altered to an opal termination codon.

Briefly, selenoproteins constitute a unique group of prokaryotic and eukaryotic polypeptides, all of which incorporate the sulfur-like element selenium as selenocysteine. Selenoproteins include bacterial format dehydrogenases, the mammalian glutathione peroxidase (GPx) family, mammalian type I iodothyronine 5′ deiodinase, and mammalian selenoprotein P (Bock et al., Trends Biochem. Sci. 16:463, 1991). All of these proteins incorporate selenocysteine cotranslationally at a UGA codon (Bock et al., Supra), formerly known only as a “stop” (or opal) codon, and utilize a unique selenocysteine-charged tRNA^{SeCYs} containing the appropriate UGA anticodon (Lee et al., J. Biol. Chem. 264:9724-9727, 1989).

For example, within one embodiment of the invention cells are grown with the toxic or ftisogenic gene in the repressed state, i.e., in the absence of selenium or with greatly reduced selenium levels compared to normal growth media (e.g., DMEM supplemented with 10% FBS). This may be achieved by adapting cells to growth in a defined media, such as IT (Insulin, transferrin; Collaborative Bioproducts, Waltham Mass.). Cells can be grown in the selenium-deficient media and when near confluency, the media is changed to either FBS containing media (FBS usually contains high quantities of selenium) or the cells moved in ITS media (Insulin, transferrin, selenium; Collaborative Bioproducts, Waltham MA). Upon selenium repletion, the mutated TGA codon is read as a selenocysteine codon and the selenocysteine incorporated into the toxic or fusogenic protein.

Although selenium responsive elements are provided herein as a representative example of translational regulatory mechanisms, it should be understood that the present invention is not so limited. In particular, a variety of other translational regulatory elements may be utilized either alone, or in combination with the above noted transcriptional elements in order control expression of a given sequence. For example, Iron response elements (IREs) occur naturally in critical proteins involved in iron metabolism. They are moderately stable stem-loop structures of approximately 26-35 nucleotides found as single copies in the 5′ or 3′ untranslated regions of these proteins (Hentze et al., Science 238:1570-1573, 1987). IREs provide the mnRNA binding site for a cytosolic protein called the IRE-binding protein (IRE-BP) (Leibold and Munro, P.N.A.S. 85:2171-2175, 1988). This protein is regulated by iron availability (Haile et al., PNAS 89:7536-7540.1992). Under conditions of iron excess, cytosolic IRE-BP do not bind IREs. Under conditions of iron deprivation, IRE-BP exhibit high affinity binding to IREs (Kd<100 pM). The switch between high and low affinity binding does not require protein synthesis and represents a posttranslational modification of the IRE-BP. The inactive protein can be activated to bind RNA with high affinity in the presence of either high concentrations of iron chelators or reducing agents, thus it appears to have a redox-sensitive sulfhiydryl switch distinguishing the two binding states.

Incorporation of IREs into the 5′ or 3′ untranslated regions of heterologous sequences such as fusogenic or toxic proteins can result in the ability to repress translation of these genes under conditions of iron deprivation. For example, the following oligonucleotide sequence or other closely related sequences (and their complements) may be synthesized with flanking restriction enzyme sites (RES) for incorporation into the 5′ untranslated region of a toxic gene or fusogenic gene such as the VSV-G gene at approximately -150 bp.

IRE sequence oligonucleotide 3 (Sequence I.D. No. 2):

5′ (RES) -CGT CGG GGT TTC CTG CTT CAA CAG TGC TTG GAC GGA ACC CGG CG-(RES) 3′

IRE sequence oligonucleotide 4 (Sequence I.D. No. 3):

5′ (RES) -CG CCG GGT TCC GTC CAA GCA CTG TTG AAG CAG GAA ACC CCG TCG-(RES) 3′

Packaging cell lines which contain IRE-VSV-G constructs may be maintained in media with low iron concentrations, with high concentrations of reducing agents such as dithiothreitol (DTT; e.g., at 100 mM), or with the iron chelator desferrioxamine at 50-100 mM. Upon addition of hemin at 50-100 mM as an iron source for 20 hours, the affinity of the IRE-BP drops and translation of VSV-G proceeds, generating up to 100-fold increase in protein expression. This therefore permits a regulatable burst of high production of VSV-G pseudotyped FIV vector.

Further mechanisms for controlled gene expression are by way of cell-permeable, small RNA or DNA molecules called aptamers, which are designed to specifically bind to certain molecules (Werstuck et al., Science 282:296-298, 1998). Aptamers can be introduced into any component of the FIV producer cell line in order to allow regulated expression of the gene of interest during vector production as well as regulation of the FIV vector transgene in the patient. One or more aptamers can be introduced in the 5′ untranslated area of the gene of interest and upon exposure to the aptamer-specific molecule, translation of the RNA will be disrupted.

### METHODS FOR UTILIZING FIV VECTOR PARTICLES

As noted above, the present invention also provides methods for delivering a selected heterologous sequence to a vertebrate or insect, comprising the step of administering to a vertebrate or insect an FIV vector particle as described herein which is capable of expressing the selected heterologous sequence. Such FIV vector particles may be administered either directly (e.g., intravenously, intramuscularly, intraperitoneally, subcutaneously, orally, rectally, intraocularly, intranasally), or by various physical methods such as lipofection (Felgner et al., PNAS 84:7413-7417, 1989), direct DNA injection (Fung et al., PNAS 80:353-357, 1983; Seeger et al., PNAS 81:5849-5852; Acsadi et al., Nature 352:815-818, 1991); microprojectile bombardment (Williams et al., PNAS 88:2726-2730, 1991); liposomes of several types (see, e.g., Wang et al., PNAS 84:7851-7855, 1987); CaPO₄ (Dubensky et al., PNAS 81:7529-7533, 1984); DNA ligand (Wu et al., J. Biol. Chem. 264:16985-16987, 1989); administration of nucleic acids alone (WO 90/11092); or administration of DNA linked to killed adenovirus (Curiel et al., Hum. Gene Ther. 3:147-154, 1992); via polycation compounds such as polylysine, utilizing receptor specific ligands; as well as with psoralen inactivated viruses such as Sendai or Adenovirus. In addition, the FIV vector particles may either be administered directly (i.e., in vivo), or to cells which have been removed (ex vivo), and subsequently returned.

As discussed in more detail below, FIV vector particles may be administered to a vertebrate or insect organism or cell for a wide variety of both therapeutic or productive purposes, including for example, for the purpose of stimulating a specific immune response; inhibiting the interaction of an agent with a host cell receptor; to express a toxic palliative, including for example, conditional toxic palliatives; to immunologically regulate the immune system; to express markers, for replacement gene therapy and/or to produce a recombinant protein. FIV vector particles may be administered to a wide variety of tissue and/or cell types, including for example, the brain and/or spinal cord (e.g., neural cells), bone marrow (e.g., hematopoietic stem cells, osteoblasts or osteoclasts, and the like), eyes (e.g., retinal cells), the liver (e.g., hepatocytes), nose, throat and lung (e.g., nasal epithelial cells), heart and blood vessels (e.g., endothelium or vascular smooth muscle cells), spleen, skin (e.g., drenditic cells or keratinocytes), blood cells (e.g., lymphocytes or portions of blood cells that have been separated from other blood cells, such as, for example, CD34 positive cells), muscles, and tumors (e.g., tumor cells). These and other uses are discussed in more detail below.

1. Immunostimulation

Within one aspect of the present invention, compositions and methods are provided for administering an FIV vector particle which is capable of preventing, inhibiting, stabilizing or reversing infectious, cancerous, auto-immune or immune diseases. Representative examples of such diseases include viral infections such as HIV, BBV, HCV, HTLV I, HTLV II, CMV, EBV, FW and BPV, melanomas, diabetes, graft vs. host disease, Alzheimer's disease and heart disease. More specifically, within one aspect of the present invention, compositions and methods are provided for stimulating an immune response (either humoral or cell-mediated) to a pathogenic agent, such that the pathogenic agent is either killed or inhibited. Representative examples of pathogenic agents include bacteria, fungi, parasites, viruses and cancer cells.

Within one embodiment of the invention the pathogenic agent is a virus, and methods are provided for stimulating a specific immune response and inhibiting viral spread by using an FIV vector particle that directs the expression of an antigen or modified form thereof to susceptible target cells capable of either (1) initiating an immune response to the viral antigen or (2) preventing the viral spread by occupying cellular receptors required for viral interactions. Expression of the vector nucleic acid encoded protein may be transient or stable with time. Where an immune response is to be stimulated to a pathogenic antigen, the FIV vector is preferably designed to express a modified form of the antigen which will stimulate an immune response and which has reduced pathogenicity relative to the native antigen. This immune response is achieved when cells present antigens in the correct manner, i.e., in the context of the MHC class I and/or II molecules along with accessory molecules such as CD3, ICAM-1, ICAM-2, LFA-1, or analogues thereof(e.g., Altmann et al., Nature 338:512, 1989). Cells infected with FIV vector particles are expected to do this efficiently because they closely mimic genuine viral infection and because they: (a) are able to infect non-replicating cells, (b) integrate into the host cell genome, (c) are not associated with any life threatening human diseases. Because of these differences, FIV vectors can easily be thought of as safe viral vectors which can be used on healthy individuals for vaccine use.

This aspect of the invention has a further advantage over other systems that might be expected to function in a similar manner, in that the presenter cells are fully viable and healthy, and low levels of viral antigens, relative to heterologous genes, are expressed. This presents a distinct advantage since the antigenic epitopes expressed can be altered by selective cloning of sub-fragments of the gene for the antigen into an FIV vector particle, leading to responses against immunogenic epitopes which may otherwise be overshadowed by immunodominant epitopes. Such an approach may be extended to the expression of a peptide having multiple epitopes, one or more of the epitopes being derived from different proteins. Further, this aspect of the invention allows efficient stimulation of cytotoxic T lymphocytes (CTL) directed against antigenic epitopes, and peptide fragments of antigens encoded by sub-fragments of genes, through intracellular synthesis and association of these peptide fragments with MHC Class I molecules. This approach may be utilized to map major immunodominant epitopes for CTL induction.

An immune response may also be achieved by transferring to an appropriate immune cell (such as a T lymphocyte) the gene for the specific T cell receptor which recognizes the antigen of interest (in the context of an appropriate MHC molecule if necessary), for an immunoglobulin which recognizes the antigen of interest, or for a hybrid of the two which provides a CTL response in the absence of the MHC context. Thus, the FIV vector particles may be used as an immunostimulant, immunomodulator, or vaccine.

In one embodiment of the invention, the FIV vector particles are delivered to dendritic cells which are the most efficient antigen-presenting cells (APC) of the immune system. In contrast to other APCs, dendritic cells are known to elicit potent primary immune responses involving naive T-cells (Weissman et al., Clin. Microbiol. Rev. 10, 358-367, 1997). The transduction of dendritic cells with FIV vector particles encoding viral or cancer immunogens may initiate a strong immune response that might be efficacious in the fight of chronic viral diseases or certain types of cancers.

In another embodiment of the invention, methods are provided for producing inhibitor palliatives wherein FIV vector particles deliver and express defective interfering viral structural proteins, which inhibit viral assembly. Such FIV vector particles may encode defective gag, pol, env or other viral particle proteins or peptides and these would inhibit in a dominant fashion the assembly of viral particles. This occurs because the interaction of normal subunits of the viral particle is disturbed by interaction with the defective subunits.

In another embodiment of the invention, methods are provided for the expression of inhibiting peptides or proteins specific for viral protease. Briefly, viral protease cleaves the viral gag and gaglpol proteins into a number of smaller peptides. Failure of this cleavage in all cases leads to complete inhibition of production of infectious retroviral particles. As an example, the HIV protease is known to be an aspartyl protease and these are known to be inhibited by peptides made from amino acids from protein or analogues. FIV vectors to inhibit HIV will express one or multiple fused copies of such peptide inhibitors.

Another embodiment involves the delivery of suppressor genes which, when deleted, mutated, or not expressed in a cell type, lead to tumorigenesis in that cell type. Reintroduction of the deleted gene by means of an FIV vector particle leads to regression of the tumor phenotype in these cells. Examples of such cancers are retinoblastoma and Wilms Tumor. Since malignancy can be considered to be an inhibition of cellular terminal differentiation compared with cell growth, administration of the FIV vector particle and expression of gene products which lead to differentiation of a tumor should also, in general, lead to regression.

In yet another embodiment, the FIV vector provides a therapeutic effect by transcribing a ribozyme (an RNA enzyme) (Haseloff and Gerlach, Nature 334:585, 1989) which will cleave and hence inactivate RNA molecules corresponding to a pathogenic function. Since ribozymes function by recognizing a specific sequence in the target RNA and this sequence is normally 12 to 17 bp, this allows specific recognition of a particular RNA species such as a RNA or a retroviral genome. Additional specificity may be achieved in some cases by making this a conditional toxic palliative (see below).

One way of increasing the effectiveness of inhibitory palliatives is to express viral inhibitory genes in conjunction with the expression of genes which increase the probability of infection of the resistant cell by the virus in question. The result is a nonproductive “dead-end” event which would compete for productive infection events. In the specific case of HIV, FIV vector particles may be delivered which inhibit HIV replication (by expressing anti-sense tat, etc., as described above) and also overexpress proteins required for infection, such as CD4. In this way, a relatively small number of vector-infected HIV-resistant cells act as a “sink” or “magnet” for multiple nonproductive fusion events with free virus or virally infected cells.

2. Blocking Agents

Many infectious diseases, cancers, autoimmune diseases, and other diseases involve the interaction of viral particles with cells, cells with cells, or cells with factors produced by themselves or other cells. In viral infections, viruses commonly enter cells via receptors on the surface of susceptible cells. In cancers or other proliferative conditions (e.g., restenosis), cells may respond inappropriately or not at all to signals from other cells or factors, or specific factors may be mutated, overexpressed, or underexpressed, resulting in loss of appropriate cell cycle control. In autoimmune disease, there is inappropriate recognition of “self” markers. Within the present invention, such interactions may be blocked by producing, in vivo, an analogue to either of the partners in an interaction. Alternatively, cell cycle control may be restored by preventing the transition from one phase to another (e.g., G1 to S phase) using a blocking factor which is absent or underexpressed. This blocking action may occur intracellularly, on the cell membrane, or extracellularly, and the action of the FIV vector particle carrying a gene for a blocking agent, can be mediated either from inside a susceptible cell or by secreting a version of the blocking protein to locally block the pathogenic interaction.

In the case of HIV, the two agents of interaction are the gp 120/gp 41 envelope protein and the CD4 receptor molecule. Thus, an appropriate blocker would be an FIV vector expressing either an HIV env analogue that blocks HIV entry without causing pathogenic effects, or a CD4 receptor analogue. The CD4 analogue would be secreted and would function to protect neighboring cells, while the gp 120/gp 41 is secreted or produced only intracellularly so as to protect only the vector-containing cell. It may be advantageous to add human immunoglobulin heavy chains or other components to CD4 in order to enhance stability or complement lysis. Administration of an FIV vector particle encoding such a hybrid-soluble CD4 to a host results in a continuous supply of a stable hybrid molecule. Efficacy of treatment can be assayed by measuring the usual indicators of disease progression, including antibody level, viral antigen production, infectious HIV levels, or levels of nonspecific infections.

In the case of uncontrolled proliferative states, such as cancer or restenosis, cell cycle progression may be halted by the expression of a number of different factors that affect signaling by cyclins or cyclin-dependent kinases (CDK). For example, the cyclin-dependent kinase inhibitors, pl6, p21, and p27 each regulate cyclin:CDK mediated cell cycle signaling. Overexpression of these factors within a cell by a FIV vector particle results in a cytostatic suppression of cell proliferation. Other factors that may be used therapeutically, as blocking agents or targets, include, for example, wild-type or mutant Rb, p53, Myc, Fos, Jun, PCNA, GAX, and p15.

3. Expression of Palliatives

Techniques similar to those described above can be used to produce FIV vector particles which direct the expression of an agent (or “palliative”) which is capable of inhibiting a function of a pathogenic agent or gene. Within the present invention, “capable of inhibiting a function” means that the palliative either directly inhibits the function or indirectly does so, for example, by converting an agent present in the cells from one which would not normally inhibit a function of the pathogenic agent to one which does. Examples of such functions for viral diseases include adsorption, replication, gene expression, assembly, and exit of the virus from infected cells. Examples of such functions for a cancerous cell, cancer-promoting growth factor, or uncontrolled proliferative condition (e.g., restenosis) include viability, cell replication, altered susceptibility to external signals (e.g., contact inhibition), and lack of production or production of mutated forms of anti-oncogene proteins.

a. Inhibitor Palliatives

In one aspect of the present invention, the FIV vector particle directs the expression of a gene which can interfere with a function of a pathogenic agent, for instance in viral or malignant diseases. Such expression may either be essentially continuous or in response to the presence in the cell of another agent associated either with the pathogenic condition or with a specific cell type (an “identifying agent”). In addition, vector delivery may be controlled by targeting vector entry specifically to the desired cell type (for instance, a virally infected or malignant cell) as discussed above.

One method of administration is leukophoresis, in which about 20% of an individual's PBLs are removed at any one time and manipulated in vitro. Thus, approximately 2×10⁹ cells may be treated and replaced. Repeat treatments may also be performed. Alternatively, bone marrow may be treated and allowed to amplify the effect as described above. In addition, packaging cell lines producing a vector may be directly injected into a subject, allowing continuous production of recombinant virions.

In one embodiment, FIV vector particles which express RNA complementary to key pathogenic gene transcripts (for example, a viral gene product or an activated cellular oncogene) can be used to inhibit translation of that transcript into protein, such as the inhibition of translation of the HIV tat protein. Since expression of this protein is essential for viral replication, cells containing the FIV vector particle would be resistant to HMV replication.

In a second embodiment, where the pathogenic agent is a single-stranded virus having a packaging signal, RNA complementary to the viral packaging signal (e.g., an HIV packaging signal when the palliative is directed against HV) is expressed, so that the association of these molecules with the viral packaging signal will, in the case of retroviruses, inhibit stem loop formation or tRNA primer binding required for proper encapsidation or replication.

In a third embodiment, FIV vector particles may be introduced which expresses a palliative capable of selectively inhibiting the expression of a pathogenic gene, or a palliative capable of inhibiting the activity of a protein produced by the pathogenic agent. In the case of HIV, one example is a mutant tat protein which lacks the ability to transactivate expression from the HIV LTR and interferes (in a transdorinant manner) with the normal functioning of tat protein. Such a mutant has been identified for HTLV II tat protein (“XII Leu⁵” mutant; see Wachsman et al., Science 235:674, 1987). A mutant transrepressor tat should inhibit replication much as has been shown for an analogous mutant repressor in HSV-1 (Friedmann et al., Nature 335:452, 1988).

Such a transcriptional repressor protein can be selected for in tissue culture using any viral-specific transcriptional promoter whose expression is stimulated by a virus-specific transactivating protein (as described above). In the specific case of HIS, a cell line expressing mHV tat protein and the HSVTK gene driven by the HIV promoter will die in the presence of ACV. However, if a series of mutated tat genes are introduced to the system, a mutant with the appropriate properties (i.e., represses transcription from the HIV promoter in the presence of wild-type tat) will grow and be selected. The mutant gene can then be reisolated from these cells. A cell line containing multiple copies of the conditionally lethal vector/tat system may be used to assure that surviving cell clones are not caused by endogenous mutations in these genes. A battery of randomly mutagenized tat genes are then introduced into these cells using a “rescuable” FIV vector (i.e., one that expresses the mutant tat protein and contains a bacterial origin of replication and drug resistance marker for growth and selection in bacteria). This allows a large number of random mutations to be evaluated and permits facile subsequent molecular cloning of the desired mutant cell line. This procedure may be used to identify and utilize mutations in a variety of viral transcriptional activator/viral promoter systems for potential antiviral therapies.

b. Conditional Toxic Palliatives

Another approach for inhibiting a pathogenic agent is to express a palliative which is toxic for the cell expressing the pathogenic condition. In this case, expression of the palliative from the FIV vector should be limited by the presence of an entity associated with the pathogenic agent, such as a specific viral RNA sequence identifying the pathogenic state, in order to avoid destruction of nonpathogenic cells.

In one embodiment of this method, FIV vector particles can be utilized to express a toxic gene (as discussed above) from a cell-specific responsive vector. In this manner, rapidly replicating cells, which contain the RNA sequences capable of activating the cell-specific responsive vectors, are preferentially destroyed by the cytotoxic agent produced by the FIV vector particle.

In a similar manner to the preceding embodiment, the FIV vector can carry a gene for phosphorylation, phosphoribosylation, ribosylation, or other metabolism of a purine- or pyrimidine-based drug. This gene may have no equivalent in mammalian cells and might come from organisms such as a virus, bacterium, fungus, or protozoan. An example of this would be the E. coli guanine phosphoribosyl transferase gene product, which is lethal in the presence of thioxanthine (see Besnard et al., Mol. Cell Biol. 7:4139-4141, 1987). Conditionally lethal gene products of this type (also referred to as “pro-drugs” or “prodrug activating enzymes”) have application to many presently known purine- or pyrimidine-based anticancer drugs, which often require intracellular ribosylation or phosphorylation in order to become effective cytotoxic agents. The conditionally lethal gene product could also metabolize a nontoxic drug which is not a purine or pyrimidine analogue to a cytotoxic form (see Searle et al., Brit. J. Cancer 53:377-384, 1986).

In another aspect of the present invention, FIV vectors are provided which direct the expression of a gene product capable of activating an otherwise inactive precursor into an active inhibitor of the pathogenic agent. For example, the HSVTK gene product may be used to more effectively metabolize potentially antiviral nucleoside analogues such as AZT or ddC. The HSVTK gene may be expressed under the control of a cell-specific responsive vector and introduced into these cell types. AZT (and other nucleoside antivirals) must be metabolized by cellular mechanisms to the nucleotide triphosphate form in order to specifically inhibit retroviral reverse transcriptase, and thus, HIV replication (Furmam et al., PNAS 83:8333-8337, 1986). Constitutive expression of HSVTK (a nucleoside and nucleoside kinase with very broad substrate specificity) results in more effective metabolism of these drugs to their biologically active nucleotide triphosphate form. AZT or ddC therapy will thereby be more effective, allowing lower doses, less generalized toxicity, and higher potency against productive infection. Additional nucleoside analogues whose nucleotide triphosphate forms show selectivity for retroviral reverse transcriptase but, as a result of the substrate specificity of cellular nucleoside and nucleotide kinases are not phosphorylated, will be made more efficacious.

Administration of these FIV vector particles to human T cell and macrophage/monocyte cell lines can increase their resistance to HIV in the presence of AZT and ddC compared to the same cells without retroviral vector treatment. Treatment with AZT would be at lower than normal levels to avoid toxic side effects but still efficiently inhibit the spread of HIV. The course of treatment would be as described for the blocker.

In one embodiment, the FIV vector particle carries a gene specifying a product which is not in itself toxic but, when processed or modified by a protein such as a protease specific to a viral or other pathogen, is converted into a toxic form. For example, the FIV vector could carry a gene encoding a proprotein for ricin A chain, which becomes toxic upon processing by the HIV protease. More specifically, a synthetic inactive proprotein form of the toxin ricin or diphtheria A chains could be cleaved to the active form by arranging for the HIV virally encoded protease to recognize and cleave off an appropriate “pro” element.

In another embodiment, the FIV vector particle may express a “reporting product” on the surface of the target cells in response to the presence of an identifying agent in the cells (such as expression of a viral gene). This surface protein can be recognized by a cytotoxic agent, such as antibodies for the reporting protein, or by cytotoxic T cells. In a similar manner, such a system can be used as a detection system (see below) to simply identify those cells having a particular gene which expresses an identifying protein.

Similarly, in another embodiment, a surface protein could be expressed which would itself be therapeutically beneficial. In the particular case of HIV, expression of the human CD4 protein specifically in HIV-infected cells may be beneficial in two ways:

1. Binding of CD4 to HIV env intracellularly could inhibit the formation of viable viral particles, much as soluble CD4 has been shown to do for free virus, but without the problem of systematic clearance and possible immunogenicity, since the protein will remain membrane bound and is structurally identical to endogenous CD4 (to which the patient should be immunologically tolerant).

2. Since the CD4/HIV env complex has been implicated as a cause of cell death, additional expression of CD4 (in the presence of excess HV-env present in HIV-infected cells) leads to more rapid cell death and thus inhibits viral dissemination. This may be particularly applicable to monocytes and macrophages, which act as a reservoir for virus production as a result of their relative refractility to HIV-induced cytotoxicity (which, in turn, is apparently due to the relative lack of CD4 on their cell surfaces).

In another embodiment, the FIV vector particle can provide a ribozyme which will cleave and inactivate RNA molecules essential for viability of the vector infected cell. By making ribozyme production dependent on a specific RNA sequence corresponding to the pathogenic state, such as HIV tat, toxicity is specific to the pathogenic state.

4. Expression of Markers

The above-described technique of expressing a palliative in a cell in response to a specific RNA sequence can also be modified to enable detection of a particular gene in a cell which expresses an identifying protein (for example, a gene carried by a particular virus), and hence enable detection of cells carrying that virus. In addition, this technique enables the detection of viruses (such as HIV) in a clinical sample of cells carrying an identifying protein associated with the virus.

This modification can be accomplished by providing a genome coding for a product, the presence of which can be readily identified (the “marker product”), in a FIV vector which responds to the presence of the identifying protein in the infected cells. For example, HIV, when it infects suitable cells, makes tat and rev. The indicator cells can thus be provided with a genome (such as by infection with an appropriate FIV virus particle) which codes for a marker gene, such as the alkaline phosphatase gene, b-galactosidase gene, or the luciferase gene which is expressed by the FIV particle upon activation by the tat and/or rev RNA transcript. In the case of b-galactosidase or alkaline phosphatase, exposing the cells to substrate analogues results in a color or fluorescence change if the sample is positive for HIV. In the case of luciferase, exposing the sample to luciferin will result in luminescence if the sample is positive for HIV. For intracellular enzymes such as b-galactosidase, the viral titer can be measured directly by counting colored or fluorescent cells, or by making cell extracts and performing a suitable assay. For the membrane bond form of alkaline phosphatase, virus titer can also be measured by performing enzyme assays on the cell surface using a fluorescent substrate. For secreted enzymes, such as an engineered form of alkaline phosphatase, small samples of culture supernatant are assayed for activity, allowing continuous monitoring of a single culture over time. Thus, different forms of this marker system can be used for different purposes. These include counting active virus, or sensitively and simply measuring viral spread in a culture and the inhibition of this spread by various drugs.

Further specificity can be incorporated into the preceding system by testing for the presence of the virus either with or without neutralizing antibodies to that virus. For example, in one portion of the clinical sample being tested, neutralizing antibodies to HV may be present; whereas in another portion there would be no neutralizing antibodies. If the tests were negative in the system where there were antibodies and positive where there were no antibodies, this would assist in confirming the presence of HIV.

Within an analogous system for an in vitro assay, the presence of a particular gene, such as a viral gene, may be determined in a cell sample. In this case, the cells of the sample are infected with a suitable FIV vector particle which carries the reporter gene which is only expressed in the presence of the appropriate viral RNA transcript. The reporter gene, after entering the sample cells, will express its reporting product (such as b-galactosidase or luciferase) only if the host cell expresses the appropriate viral proteins.

These assays are more rapid and sensitive, since the reporter gene can express a greater amount of reporting product than identifying agent present, which results in an amplification effect.

5. Immune Down-Regulation

As described above, the present invention also provides FIV vector particles capable of suppressing one or more elements of the immune system in target cells infected with the FIV vector particles. Briefly, specific down-regulation of inappropriate or unwanted immune responses, such as in chronic hepatitis or in transplants of heterologous tissue such as bone marrow, can be engineered using immune-suppressive viral gene products which suppress surface expression of transplantation (MHC) antigen. Group C adenoviruses Ad2 and Ad5 possess a 19 kd glycoprotein (gp 19) encoded in the E3 region of the virus. This gp 19 molecule binds to class I MHC molecules in the endoplasmic reticulum of cells, and prevents terminal glycosylation and translocation of class I MHC to the cell surface. For example, prior to bone marrow transplantation, donor bone marrow cells may be infected with a gp 19-encoding FIV vector which, upon expression of the gp 19, inhibit the surface expression of MHC class I transplantation antigens. These donor cells may be transplanted with low risk of graft rejection and may require a minimal immunosuppressive regimen for the transplant patient. This may allow an acceptable donor-recipient chimeric state to exist with fewer complications. Similar treatments may be used to treat the range of so-called autoimmune diseases, including lupus erythromiatis, multiple sclerosis, rheumatoid arthritis or chronic hepatitis B infection.

An alternative method involves the use of anti-sense message, ribozyme, or other specific gene expression inhibitor specific for T cell clones which are autoreactive in nature. These block the expression of the T cell receptor of particular unwanted clones responsible for an autoimmune response. The anti-sense, ribozyme, or other gene may be introduced using the FIV vector delivery system.

6. Replacement or Augmentation Gene Therapy

One further aspect of the present invention relates to transforming cells of a vertebrate or insect with a FIV vector which supplies genetic sequences capable of expressing a therapeutic protein. Within one embodiment of the present invention, the FIV vector is designed to express a therapeutic protein capable of preventing, inhibiting, stabilizing or reversing an inherited or noninherited genetic defect in metabolism, immune regulation, hormonal regulation, enzymatic or membrane associated structural function. This embodiment also describes the FIV vector particle capable of transducing individual cells, whereby the therapeutic protein is able to be expressed systemically or locally from a specific cell or tissue, whereby the therapeutic protein is capable of (a) the replacement of an absent or defective cellular protein or enzyme, or (b) supplement production of a defective of low expressed cellular protein or enzymne. Such diseases may include cystic fibrosis, Parkinson's disease, hypercholesterolemia, adenosine deaminase deficiency, β-globin disorders, Hemophilia A & B, Gaucher's disease, diabetes and leukemia.

a. Treatment of Gaucher disease

As an example of the present invention, FIV vector particles can be constructed and utilized to treat Gaucher disease. Briefly, Gaucher disease is a genetic disorder that is characterized by the deficiency of the enzyme glucocerebrosidase. This type of therapy is an example of a single gene replacement therapy by providing a functional cellular enzyme. This enzyme deficiency leads to the accumulation of glucocerebroside in the lysosomes of all cells in the body. However, the disease phenotype is manifested only in the macrophages, except in the very rare neuronpathic forms of the disease. The disease usually leads to enlargement of the liver and spleen and lesions in the bones. (For a review, see Science 256:794, 1992, and The Metabolic Basis of Inherited Disease, 6th ed., Scriver et al., vol. 2, p. 167⁷).

b. FIV vector particles Expressing Human Factor VIII and Factor IX for Treatment of Hemophilia

Within one embodiment of the invention, FIV vector particles expressing a B-domain deleted factor VIII protein are provided (see also PCT WO 91/09122, and Attorney's Docket No. 1155.005 entitled “Methods for Administration of Recombinant Gene Delivery Vehicles for Treatment of Hemophilia and Other Disorders”).

Briefly, the B domain separates the second and third A domains of factor FVIII in the newly synthesized single-chain molecule. The B domain extends from amino acids 712 to 1648 according to Wood et al., 1984, Nature 312:330-337. Proteolytic activation of factor VIllI involves cleavage at specific Arg residues located at positions 372, 740, and 1689. Cleavages of plasma factor VIII by thrombin or Factor Xa at Arg 372 and Arg 1689 are essential for factor VIII to participate in coagulation. Therefore, activated factor VIII consists of a heterodimer comprising amino acids residues 1-372 (containing the Al domain) and residues 373-740 (containing the A2 domain), and residues 1690-2332 (containing the A3-C1-C2 domain).

An important advantage in using the B domain deleted FVIII molecule is that the reduced size appears to be less prone to proteolytic degradation and therefore, no addition of plasma-derived albumin is necessary for stabilization of the final product. The term “B domain deletion” as used herein with respect to factor VIII protein refers to a factor VIII protein in which some or all removal of some or all of the amino acids between residues 711 and 1694 have been deleted, and which still preserves a biologically active FVIII molecule.

A range of B domain deletions can exist depending on which amino acid residues in the B domain is deleted and whereby the biological activity of the FVIII molecule is still preserved. A specific B domain deletion called the SQN exists which is created by fusing Ser 743 to Gln 1638 (Lind et al., 1995, Eur J. Biochem 323:19-27, and PCT WO 91/09122) This deletes amino acid residues 744 to 1637 from the B domain creating a Ser-Glu-Asn (SQN) link between the A2 and A3 FVIII domains. When compared to plasma-derived FVIII, the SQN deletion of the B domain of FVIII did not influence its in vivo pharmacokinetics (Fijnvandraat, et al., P.R. Schattauer Vertagsgesellschatt mbH (Stuttgart) 77:298-302, 1997). The terms “Factor VIII SQN deletion” or “SQN deletion” as used herein refer to this deletion and to other deletions which preserve the single S-Q-N tripeptide sequence and which result in the deletion of the amino acids between the two B-domain SQN sequences (See PCT WO 91/09122 for a description of this amino acid sequence).

There are number of other B-domain deleted forms of factor VIII. cDNA's encoding all of these B-domain deleted factor VIII proteins can be inserted into FIV vector particles by using standard molecular biology techniques. For example cDNA molecules encoding the following B-domain factor VIII deletions can be constructed as described below:

| | |
|---|---|
| Eaton (1986) Biochemistry | des 797-1562 deletion |
| 25:8343 | |
| Toole (1986) PNAS 83:5939 | des 760-1639 (LA-FVIII) |
| Meutien (1988) Prot Eng 2:301 | des 771-1666 |
| | (FVIII del II: missing one |
| | thrombin site) |
| Sarver (1987) DNA 6:553 | des 747-1560 |
| Mertens (1993) Br J Haematol | des 868-1562 |
| 85:133 | des 713-1637 (thrombin resistant) |
| Esmon (1990) Blood 76:1593 | des 797-1562 |
| Donath (1995) Biochem J | des 741-1668 |
| 312:49 | |
| Webb (1993) BBRC 190:536 | PCR cloned from mRNA |
| Lind (1995) Eur J Biochem | des 748-1648 (partially |
| 232:19 | processed) |
| | des 753-1648 (partially processed) |
| | des 777-1648 (partially processed) |
| | des 744-1637 (FVIII-SQ) |
| | des 748-1645 (FVIII-RH) |
| | des |
| | B-domain + |
| | 0, 1 ,2 Arg |
| | (partially |
| | processed) |
| | desB, +3 Arg (FVIIIR4) |
| | desB, +4 Arg (FVIIIR5) |
| Langner (1988) Behring Inst | des 741-1689 |
| Mitt 16-25 | des 816-1598 |
| Cheung (1996) Blood 88:325a | des 746-1639 |
| Pipes (1996) Blood 88:441a | des 795-1688 (thrombin sites |
| | mutated) |

A B domain deletion in which an IgG hinge region has been inserted can also be used. For instance, a deletion of this type can be obtained from plasmid PSVF8-tb2, which was designed to link the heavy and light chains with a short hinge region from immunoglobulin A. To obtain cleavage at the end of the heavy chain and to release the light chain, some residues of the b domain are included on either side of the hinge sequence. The 5′ untranslated leader and signal peptide are from the human Factor VIII:C cDNA, with the Kozak consensus sequence at the initiation codon as in pSVF8-302. A description of this vector is included in Chapman et al., U.S. Pat. No. 5,595,886. The 3′ untranslated region is the same fused Factor VIII and tPA sequence as found in pSVF8-80K.

The construction may be completed in two steps: an oligomer with cohesive ends for EcoRI and BclI (117 bp) was cloned into a transfer vector, pF8GM7, the DNA sequence of the oligomer was checked by ml3 subcloning and Sanger sequencing.

Next, the final plasmid was assembled by ligation of the following three fragments:

(a) FspI-EcoRI fragment form pSVF8-92S;

(b) EcoRI-NdeI fragment of the transfer vector pF8GM7 with oligomer; and

(c) FspI-NdeI fragment of pSVF8-80K.

Descriptions of pSVF8-92S and pSVF8-80K are included in Chapman et al., U.S. Pat. No. 5,595,886.

Three additional B domain-deleted factor VIII constructs of particular interest for inclusion in the FIV vector particles of the invention can be prepared as follows. Plasmid pSVF8-500 encodes a factor VIII protein with amino acids 770 to 1656 of the full length Factor VIII deleted. In addition the threonine at position 1672 of the full-length factor VIII sequence was also deleted. The following is a description of the construction of the vector.

The pSVF8-500 plasmid is a derivative of pSVF8-302 in which the regions coding for the 92K and 80K domains are fuised with a small connecting b-region of 21 amino acids, retaining the natural proteolytic processing sites. This plasmid was constructed in the following manner:

(1) A SalI-KpnI fragment of 1984 bp containing the region coding for the 92K protein (except for the carboxyl terminal end) and BstM-SalI fragment of 2186 bp containing the region coding for the carboxyl end of the 80K protein with 3′ end untranslated region were isolated by gel electrophoresis after digestion of pSVF8-302 with restriction enzymes.

(2) A BclI-BstXI fragment of 1705 bp containing most of the region coding for the 80K protein was isolated after gel electrophoresis of the BamHI-Xbal fragment of pUC12F8. (pUCF812 is prepared from pF8-102 which is described in U.S. Pat. No. 5,045,455. pF8-102 is digested with Bam-XhaI and ligated into vector pUC12 by in vitro mutagenesis at a Bcil site using the following primer: 5′ ACT ACT CTT CAA TCT GAT CAA GAG GAA 3′ (Seq. ID No. 4).

(3) A Kpnl-EcoRI fragment containing the carboxyl end of the 92K protein and part of the b region (4 amino acids) was obtained by digestion of the Sall cassette from pSVF8-302 with KpnI and EcoRI.

(4) Ligation of four pieces of synthetic DNA (shown in FIG. 39) to the fragments of steps (2) and (3) and digestion with KpnI.

(5) Final ligation of fragments from steps (1) and (4); digestion with SalI and gel purification of the 6428 bp SalI cassette.

(6) Ligation of the SalI cassette into pSV7d vector; transformation of BBIO1 and colony hybridization to isolate pSVF8-500 (FIG. 40). The sequence of the junction region coding for 92K-b-80K was verified by DNA sequence after cloning in M13.

The sequence was changed to incorporate unique NruI and Mul restriction sites without changing the amino acid sequence. These sites were also used to construct other two additional B-domain deleted vectors which are described below. pSV500BDThr was constructed from pSVF8-500. The threonine deletion at position 1672 was maintained. A synthetic linker was used to construct pSV500BDThr. The linker extends from a unique NruI site at Ser(765) to a unique MluI site at Ile(1659) in the pSVF8-500 vector. This linker was substituted for the corresponding region of pSVF8-500.

A third vector pSVF8-500B was constructed from pSV500BDThr. This vector is identical to pSVF8-500B except that the codon for threonine 1672 was re-inserted using standard mutagenesis methods. The relationship between, pSVF8-500B, pSVF8-500B, is further illustrated in the table below. Amino acid sequence numbers in the table were determined by reference to fijll-length factor VIII sequence.

| | Amino Acids | Thr at 1672 |
|---|---|---|
| Name | Deleted | Deletion |
| pSVF8-500 | 770 to 1656 | Yes |
| pSVF8- | 779 to 1658 | Yes |
| 500BDThr | | |
| pSVF80-500B | 779 to 1658 | No |

In all cases, the BglII-PflI 1.35 kb fragments of each modified cDNA listed above can be inserted into the FIV vector particles described herein using standard molecular biology procedures known to those of skill in the art and described herein.

The full-length factor VIII cDNA can also be inserted into the FIV vector particles of the invention (see, e.g., WO 96/21035). A variety of Factor VIII deletions, mutations, and polypeptide analogs of Factor VIII can also be introduced into the FIV vector particles of the invention including FIV vector particles by modifications of the procedures described herein. These analogs include, for instance, those described in PCT Patent Publication Nos. WO 97/03193, WO 97/03194, WO 97/03195, and WO 97/03191, all of which are hereby incorporated by reference.

Hemophilia B can also be treated with systemically administered factor IX-expressing FIV vector particles including FIV vector particles. Human factor IX deficiency (Christmas disease or Hemophilia B) affects primarily males because it is transmitted as sex-linked recessive trait. It affects about 2000 people in the US. The human factor gene codes a 416 amino acids of mature protein.

The human factor IX cDNA can be obtained for instance by constructing plasmid pHfIX1, as described by Kurachi and Davie, 1982, PNAS 79(21):6461-6464. The cDNA sequence can be excised as a PstI fragment of about 1.5 kb, blunt ended using T4 DNA polymerase. The factor cDNA fragment can be readily inserted, for example into a Srfl site introduced into a FIV vector particle.

c. FIV vector particles expressing other clotting factors

i. Factor V

FIV vector particles can be constructed using molecular biology techniques known to those of skill in the art. For instance, Factor V cDNA is obtained from pMT2-V (Jenny, 1987, PNAS 84:4846; ATCC deposit #40515) by digestion with SalI. The 7 kb cDNA band is excised from agarose gels and cloned into FIV vector particles, using standard molecular biology techniques.

Either a full-length or a B-domain deletion or substitution of the factor V cDNA can be expressed by the gene therapy vectors of the invention. Factor V B-domain deletions such as those reported by Marquette, 1995, Blood 86:3026, and Kane, 1990, Biochemistry 29:6762, can be made as described by these authors.

ii. Antithrombin III

FIV vector particles capable of expressing ATIII cDNA can be readily constructed using standard molecular biology techniques known to those of skill in the art. For instance a FIV vector particle expressing AT III can be constructed from the vector pKT218 (Prochownik, 1983, J. Biol. Chem. 258:8389; ATCC number 57224/57225) by excision with PstI. The 1.6 kb cDNA insert can be recovered from agarose gels and cloned into the PstI site of vector SK-. The insert can be recovered by restriction enzyme digestion and cloned into FIV vector particles described herein by the restriction enzymes.

iii. Protein C

The FIV vector particles of the invention capable of expressing Protein C can be made using a wide variety of techniques given the present disclosure. For instance, protein C cDNA will be obtained by restriction enzyme digestion of published vector (Foster, 1984, PNAS 81:4766; Beckmann, 1985, Nucleic Acids Res 13:5233). The 1.6 kb cDNA insert can be recovered from agarose gels and cloned into the multiple cloning site of vector SK- under standard conditions. The insert can be recovered by restriction enzyme digestion and cloned into a FIV vector; for example, excision by XhoI/Notl digestion followed by cloning into XhoI/NotI digested FIV vector.

iv. Prothrombin

FIV vector particles expressing prothrombin and its variants can be constructed by methods known to those of skill in the art, by using variations on the methods described herein. For instance, prothrombin cDNA can be obtained by restriction enzyme digestion of a published vector (Degen (1983) Biochemistry 22:2087). The 1.9 kb cDNA insert can be recovered from agarose gels and cloned into the multiple cloning site of vector SK-. The insert can be recovered by restriction enzyme digestion and cloned into a FIV vector using restriction enzyme digestion

v. Thrombomodulin

FIV vector particles expressing thrombomodulin and its variants can be constructed using techniques known to those of skill in the art. For instance, thrombomodulin cDNA can be obtained from the vector pucl9TM15 (Jackman, 1987, PNAS 84:6425; Shirai, 1988, J. Biochem. 103:281; Wen, 1987, Biochemistry 26:4350; Suzuki, 1987, EMBO J. 6:1891; ATCC number 61348,61349) by excision with SalI. The 3.7 kb cDNA insert can be recovered from agarose gels and cloned into the SalI site of lentiviral vector.

d. FIV vector particles treatment of hereditary disorders and other conditions

There are a number of proteins useful for treatment of hereditary disorders that can be expressed in vivo by the methods of invention. Many genetic diseases caused by inheritance of defective genes result in the failure to produce normal gene products, for example, thalassemia, phenylketonuria, Lesch-Nyhan syndrome, severe combined immunodeficiency (SCID), hemophilia, A and B, cystic fibrosis, Duchenne's Muscular Dystrophy, inherited emphysema and familial hypercholesterolemia (Mulligan et al., 1993, Science 260:926; Anderson et al., 1992, Science 256:808; Friedman et al., 1989, Science 244:1275). Although genetic diseases may result in the absence of a gene product, endocrine disorders, such as diabetes and hypopituitarism, are caused by the inability of the gene to produce adequate levels of the appropriate hormone insulin and human growth hormone respectively.

Gene therapy by the methods of the invention is a powerful approach for treating these types of disorders. This therapy involves the introduction of normal recombinant genes into somatic cells so that new or missing proteins are produced inside the cells of a patient. A number of genetic diseases can be treated by gene therapy, including adenine deaminase deficiency, cystic fibrosis, a₁-antitrypsin deficiency, Gaucher's syndrome, as well as non-genetic diseases.

Other representative diseases include β-glucuronidase for treatment of the storage disease mucopolysaccharidosis type VII (Russell et al., Gut 9:585-589, 1968; Sands et al., Neuromuscul. Disord. 7:352-360, 1997), lactase for treatment of hereditary lactose intolerance, AD for treatment of ADA deficiency, and alpha-i antitypsin for treatment of alpha- I antitrypsin deficiency. See F.D. Ledley, 1987, J. Pediatics 110: 157-174; I. Verma, Scientific American (Nov., 1987) pp. 68-84; and PCT Patent Publication WO 95/27512 entitled “Gene Therapy Treatment for a Variety of Diseases and Disorders” for a description of gene therapy treatment of genetic diseases.

One such disorder is familial hypercholesterolemia is a disease characterized clinically by a lifelong elevation of low density lipoprotein (LDL), the major cholesterol-transport lipoprotein in human plasma; Pathologically by the deposition of LDL-derived cholesterol in tendons, skin and arteries leading to premature coronary heart disease; and genetically by autosomal dominant inherited trait. Heterozygotes number about 1 in 500 persons worldwide. Their cells are able to bind cholesterol at about half the rate of normal cells. Their plasma cholesterol levels show two fold elevation starting at birth. Homozygotes number 1 in 1 million persons They have severe cholesterolemia with death occurring usually before age 20. The disease (Arteriosclerosis) depends on geography. It affects 15.5 per 100,000 individuals in the U.S. (20,000 total) and 3.3 per 100,000 individuals in Japan. FIV vector particles expressing the LDL receptor for treatment of disorders manifesting with elevated serum LDL can be constructed by techniques known to those of skill in the art.

There are a variety of other proteins of therapeutic interest that can be expressed in vivo by FIV vector particles using the methods of the invention. For instance sustained in vivo expression of tissue factor inhibitory protein (TFPI) is useful for treatment of conditions including sepsis and DIC and in preventing reperfusion injury. (See PCT Patent Publications Nos. WO 93/24143 ,WO 93/25230 and WO 96/06637. Nucleic acid sequences encoding various forms of TFPI can be obtained, for example, as described in US Patent Nos. 4,966,852; 5,106,833; and 5,466,783, and can be incorporated in FIV vector as is described herein.

Further proteins of therapeutic interest for the treatment of coronary heart disease and congestive heart failure are fibroblast growth factors such as e.g., FGF-2 (Suzuki et al., Biochem. Biophys. Res. Commun 186:1192-1200, 1992).

Other proteins of therapeutic interest such as erythropoietin (EPO) and leptin can also be expressed in vivo by FIV vector particles according to the methods of the invention. For instance EPO is useful in gene therapy treatment of a variety of disorders including anemia (see PCT publication number WO 95/13376 entitled “Gene Therapy for Treatment of Anemia”.) Sustained gene therapy delivery of leptin by the methods of the invention is usefull in treatment of obesity. (See WO 96/05309 entitled “Obesity Polypeptides able to modulate body weight” for a description of the leptin gene and its use in the treatment of obesity. FIV vector particle expressing EPO or leptin can readily be produced using the methods described herein and the constructs described in these two patent publications.

A variety of other disorders can also be treated by the methods of the invention. For example, sustained in vivo systemic production of apolipoprotein E or apolipoprotein A by the FIV vector particles of the invention can be used for treatment of hyperlipidemia. (See Breslow, J. et al. Biotechnology 12, 365 (1994).) In addition, sustained production of angiotensin receptor inhibitor (T. L. Goodfiriend, et al., 1996, N. Engl. J. Med. 334:1469) can effected by the gene therapy methods described herein. As yet an additional example, the long term in vivo systemic production of angiostatin by the lentiviral vector particles of the invention is useful in the treatment of a variety of tumors. (See O'Reilly et al., 1996, Nature Med. 2:689.

7. Lymphokines and Lvmphokine Receptors

As noted above, the present invention also provides FIV vector particles which can, among other functions, direct the expression of one or more cytokines or cytokine receptors. Briefly, in addition to their role as cancer therapeutics, cytokines can have negative effects resulting in certain pathological conditions. For example, most resting T-cells, B cells, large granular lymphocytes and monocytes do not express IL-2R (receptor). In contrast to the lack of IL-2R expression on normal resting cells, IL-2R is expressed by abnormal cells in patients with certain leukemias (Am, Hairy-cell, Hodgkins, acute and chronic granulocytic), autoimmune diseases, and is associated with allograft rejection. Interestingly, in most of these patients the serum concentration of a soluble form of IL-2R is elevated. Therefore, with certain embodiments of the invention therapy may be effected by increasing the serum concentration of the soluble form of the cytokine receptor. For example, in the case of IL-2R, a FIV vector can be engineered to produce both soluble IL-2R and IL-2R, creating a high affinity soluble receptor. In this configuration, serum IL-2 levels would decrease, inhibiting the paracrine loop. This same strategy also may be effective against autoimmune diseases. In particular, because some autoimmune diseases (e.g., Rheumatoid arthritis, SLE) also are associated with abnormal expression of IL-2, blocking the action of IL-2 by increasing the serum level of receptor may also be utilized in order to treat such autoimmune diseases.

In other cases inhibiting the levels of IL-1 may be beneficial. Briefly, IL-1 consists of two polypeptides, IL-1 and IL-1, each of which has pleiotropic effects. IL-1 is primarily synthesized by mononuclear phagocytes, in response to stimulation by microbial products or inflammation. There is a naturally occurring antagonist of the IL-1R, referred to as the IL-1 Receptor antagonist (“IL-1Ra”). This IL-1R antagonist has the same molecular size as mature IL-1 and is structurally related to it. However, binding of IL-iRa to the IL-1R does not initiate any receptor signaling. Thus, this molecule has a different mechanism of action than a soluble receptor, which complexes with the cytokine and thus prevents interaction with the receptor. IL-1 does not seem to play an important role in normal homeostasis. In animals, antibodies to IL-1 receptors reduce inflammation and anorexia due to endotoxins and other inflammation inducing agents.

In the case of septic shock, IL-1 induces secondary compounds which are potent vasodilators. In animals, exogenously supplied IL-1 decreases mean arterial pressure and induces leukopenia. Neutralizing antibody to IL-1 reduced endotoxin-induced fever in animals. In a study of patients with septic shock who were treated with a constant infusion of IL-1R for three days, the 28 day mortality was 16% compared to 44% in patients who received placebo infusions. In the case of autoimmune disease, reducing the activity of IL-1 reduces inflammation. Similarly, blocking the activity of IL-1 with recombinant receptors can result in increased allograft survival in animals, again presumably by decreasing inflammation.

These diseases provide further examples where FIV vector particles may be a engineered to produce a soluble receptor or more specifically the IL-1Ra molecule. For example, in patients undergoing septic shock, a single injection of IL-1Ra producing vector particles could replace the current approach requiring a constant infusion of recombinant IL-1R.

Cytokine responses, or more specifically, incorrect cytokine responses may also be involved in the failure to control or resolve infectious diseases. Perhaps the best studied example is non-healing forms of leishmaniasis in mice and humans which have strong, but counterproductive T_{H}2-dominated responses. Similarly, lepromotomatous leprosy is associated with a dominant, but inappropriate T_{H}2 response. In these conditions, FIV vector particles may be useful for increasing circulating levels of ON gamma, as opposed to the site-directed approach proposed for solid tumor therapy. IFN gamma is produced by T_{H}-1 T-cells, and finctions as a negative regulator of T_{H}-2 subtype proliferation. IFN gamma also antagonizes many of the IL-4 mediated effects on B-cells, including isotype switching to IgE.

IgE, mast cells and eosinophils are involved in mediating allergic reaction. IL-4 acts on differentiating T-cells to stimulate T_{H}-2 development, while inhibiting T_{H}-1 responses. Thus, FIV-based gene therapy may also be accomplished in conjunction with traditional allergy therapeutics. One possibility is to deliver FIV vector particles which produces IL4R with small amounts of the offending allergen (i.e., traditional allergy shots). Soluble IL-4R would prevent the activity of IL-4, and thus prevent the induction of a strong T_{H}-2 response.

a. FIV vector particlesfor treatment of viral hepatitis

The FIV vector particles including FIV vectors and the methods of administration described are useflul for treatment of viral hepatitis, including hepatitis B and hepatitis C. For instance, the FIV vector particles of the invention can be used to express interferon-alpha for treatment of viral hepatitis. While not wishing to be bound by theory, FIV vector particles injected intravenously preferentially transduce liver cells. Thus, the methods of intravenous delivery described herein for FIV vector particles can be used for treatment of liver diseases such as hepatitis and in particular viral hepatitis, in which therapeutic proteins expressed by the FIV vector particles can be delivered preferentially to the liver.

Currently, the only approved treatment for chronic hepatitis B, C and D infections is the use of alpha interferon 2a and 2b. Alpha-interferon is a secreted protein induced in B lymphocytes, macrophages and null lymphocytes by foreign cells, virus-infected cells, tumor cells, bacterial cells and products and viral envelopes. The mechanism of antiviral action of interferon is by inducing the synthesis of effector proteins: two of the most important are 2′, 5′-oligo-adenylate synthetase (OAS) and dsRNA-dependent protein kinase (RDPK). OAS synthesizes adenylate oligomers that activate RNAaseL, which degrades viral single stranded RNA. RDPK phosphorylates initiation factor eIF-2a which results in the inhibition of viral protein translation. In addition to the direct antiviral effect, alpha interferon has immunomodulatory effects that are important against viral infections. These immunomodulatory effects are: enhancement of the expression of both Class I and class II major histocompatibility complex (MHC) molecules, modulation of the expression of the interleukin-2 receptor, TNF-a receptor, transferrin receptor, enhancement of spontaneous natural killer (NK) cell cytotoxicity and modulation of antibody production by B cells. In chronic hepatitis B infection, the beneficial effect of interferon alpha appears to be from the immunomodulatory effects, while in chronic hepatitis C infection, the beneficial effect is dependent on its antiviral activity. (Bresters, D., in Hepatitis C Virus, ppl2l-136, Reesink H W (ed), 1994). The mechanism of action in interferon alpha for treatment of chronic hepatitis D is poorly understood (Rizzetto, M. and Rosina, F. in Viral Hepatitis, pp. 363-369, Zuckerman, A. J. and Thomas H. C. (ed), 1993).

Localized expression of interferon alpha in the liver from a FIV vector particle can be an effective treatment for hepatitis. While not wishing to bound by theory, delivery of alpha interferon at the site of infection by the gene therapy vectors of the invention, including FIV vector particles, results in high local concentration of the cytokine thereby focusing the antiviral and immunological effects to the adjacent infected hepatocytes. A further advantage of this treatment is that the current systemic mode of systemic alpha interferon therapy may either be unnecessary or be reduced in dose and frequency of treatment. This reduction can reduce the adverse side effects associated with the systemic delivery of alpha interferon. Thus, the gene therapy approaches described herein may be used in combination with administration of alpha-interferon protein formulations.

The construction of a number of different FIV vector particles expressing interferon-alpha can be readily accomplished given the disclosure provided herein. There are at least 24 different human alpha interferon genes or pseudogenes. There are two distinct families (I and II); mature human alpha interferon (I) are 166 amino acids long (one is 165 amino acids ) whereas alpha interferon (II) have 172 amino acids. Eighteen genes are in the alpha interferon I family, including at least four pseudogenes. Six genes are in the alpha interferon II family, including five pseudogenes (Callard, R., and Gearing, A., Cytokine Facts Book, Academic Press, 1994 pp. 148-154). In Example 33 herein, we use alpha interferon 2a, 2b, 2c, 54 and 76, all members of the alpha interferon (I) family. Similar techniques can be used for inserting other members of the alpha interferon I family (such as alpha interferon F and N) into lentiviral vector particles. Thus other biologically active forms of alpha-interferon in addition to 2a, 2b, 2c, 54 and 76 as described herein can also be expressed by the FIV vector particles of the invention and used for treatment of viral hepatitis.

Patients with viral hepatitis can be treated a combination gene therapy approach. A FIV vector particle expressing a protein drug such as alpha-interferon can be administered intravenously or directly to the liver by methods described herein. This therapeutic approach can be combined with intramusuclar delivery of a FIV vector particle expressing a hepatitis B or hepatitis C antigen for inducing a immune response against the hepatitis virus. Specific hepatitis B and C antigens useful in this type of therapy and the construction of FIV vector particles expressing such antigens are described herein and in PCT Patent Publication No. WO 93/15207. In addition, molecularly cloned genomes which encode the hepatitis B virus may be obtained from a variety of sources including, for example, the American Type Culture Collection (ATCC, Rockville, Md.). For example, ATCC No. 45020 contains the total genomic DNA of hepatitis B (extracted from purified Dane particles) (see FIG. 3 of Blum et al., 1989, TIG 5(5):154-158) in the Bam HI site of pBR322 (Moriarty etal., 1981, PNAS 78:2606-2610). (Note that correctable errors occur in the sequence of ATCC No. 45020.)

8. Suicide Vectors

One further aspect of the present invention relates to the use of FIV suicide vectors to limit the spread of wild-type lentivirus in the packaging/producer cell lines. For example, within one embodiment the FIV vector particles contains a prodrug activating enzyme as discussed above which, upon administration of the prodrug (e.g., gancyclovir) results in the death of cells containing the vector particles.

9. FIV vectors to Prevent the Spread of Metastatic Tumors

One further aspect of the present invention relates to the use of FIV vector particles for inhibiting or reducing the invasiveness of malignant neoplasms. Briefly, the extent of malignancy typically relates to vascularization of the tumor. One cause for tumor vascularization is the production of soluble tumor angiogenesis factors (TAF) (Paweletz et al., Crit. Rev. OncoL Hematol. 9:197, 1989) expressed by some tumors. Within one aspect of the present invention, tumor vascularization may be slowed utilizing FIV vectors to express antisense or ribozyme RNA molecules specific for TAF. Alternatively, anti-angiogenesis factors (Moses et al., Science 248:1408, 1990; Shapiro et al., PNAS 84:2238, 1987) may be expressed either alone or in combination with the above-described ribozymes or antisense sequences in order to slow or inhibit tumor vascularization. Within one embodiment, the anti-angiogenesis molecule 16K (Clapp et al., Endocrinology 133:1292, 1993; Ferrara et al., Endocrinology 129:896, 1991) can be delivered by an FIV vector. Alternatively, FIV vector particles can also be used to express an antibody specific for the TAF receptors on surrounding tissues.

10. Modulation of Transcription Factor Activity

In yet another embodiment, FIV vector particles may be utilized in order to regulate the growth control activity of transcription factors in the infected cell. Briefly, transcription factors directly influence the pattern of gene expression through sequence-specific trans-activation or repression (Karin, New Biologist 21:126-131, 1990). Thus, it is not surprising that mutated transcription factors represent a family of oncogenes. FIV vector particles can be used, for example, to return control to tumor cells whose unregulated growth is activated by oncogenic transcription factors, and proteins which promote or inhibit the binding cooperatively in the formation of homo- and heterodimer trans-activating or repressing transcription factor complexes.

One method for reversing cell proliferation would be to inhibit the trans-activating potential of the c-mycIMax heterodimer transcription factor complex. Briefly, the nuclear oncogene c-myc is expressed by proliferating cells and can be activated by several distinct mechanisms, including retroviral insertion, amplification, and chromosomal translocation. The Max protein is expressed in quiescent cells and, independently of c-myc, either alone or in conjunction with an unidentified factor, functions to repress expression of the same genes activated by the myc/Max heterodimer (Cole, Cell 65:715-716, 1991).

Inhibition of c-myc or c-myc/Max proliferation of tumor cells may be accomplished by the overexpression of Max in target cells controlled by FIV vectors. The Max protein is only 160 amino acids (corresponding to 480 nucleotide RNA length) and is easily incorporated into a FIV vector either independently, or in combination with other genes and/or antisense/ribozyme moieties targeted to factors which release growth control of the cell.

Modulation of homo/hetero-complex association is another approach to control transcription factor activated gene expression. For example, transport from the cytoplasm to the nucleus of the trans-activating transcription factor NF-B is prevented while in a heterodimer complex with the inhibitor protein IB. Upon induction by a variety of agents, including certain cytokines, IB becomes phosphorylated and NF-B is released and transported to the nucleus, where it can exert its sequence-specific trans-activating function (Baeuerle and Baltimore, Science 242:540-546, 1988). The dissociation of the NF-B/IB complex can be prevented by masking with an antibody the phosphorylation site of IB. This approach would effectively inhibit the trans-activation activity of the NF-IB transcription factor by preventing its transport to the nucleus. Expression of the EB phosphorylation site specific antibody or protein in target cells may be accomplished with a FIV gene transfer vector. An approach similar to the one described here could be used to prevent the formation of the trans-activating transcription heterodimer factor AP-1 (Turner and Tijan, Science 243:1689-1694, 1989), by inhibiting the association between thejun andfos proteins.

11. FIV vector particle delivery to cats

In one embodiment of the present invention, FIV vector particles are used to deliver heterologous genes to cats. Gene delivery to cats using the cat-specific delivery system based on FIV can be used for various purposes and establishes and small animal model where many applications and parameters of gene delivery can be easily studied in an in vivo situation.

Within one aspect of the invention, FIV vector particles are used for veterinary applications by introducing heterologous genes to cats in order to vaccinate for various feline diseases and/or deliver therapeutic genes to improve the health for genetic disorders, cancers or viral diseases of cats. The efficiency and level of gene expression in cats is expected to be very high since the heterologous gene is driven by the FIV LTR. Therefore, this gene delivery approach might have an advantage over existing methods of vaccination and/or introduction of heterologous genes into cats.

Within another aspect of the invention, marking and repopulation studies are carried out in a cat model after transduction of hematopoietic cells.

Furthermore, the implications of certain heterologous genes that might help fight FIV disease (e.g., antisense DNA sequences, cytokines) are introduced with the FIV vector particles and studied in the feline system. The FIV disease progression in cats is very similar to the HIV disease progression in humans. This small animal model might therefore give valuable insight in possible treatments of HIV. Furthermore, the effectiveness of various attenuated FIV viruses can easily be studied in cats and might lead to the development of attenuated HIV viruses that effectively protect the host to new wildtype virus challenge.

Within another aspect, FIV vector particles are used to deliver genes to feline dendritic cells. Using this cat model, an in vivo comparative study of the potential to present antigen and elicit efficacious immune responses of dendritic cells versus other APCs can be examined. Theses studies might give valuable insight into the function of the immune system and allow an analysis of various parameters of gene delivery (e.g., type of antigen, dose, route of delivery, time course) in an in vivo situation.

### FORMULATION

Within other aspects of the present invention, methods are provided for concentrating or purifying, and for preserving infectious FIV vector particles, such that the FIV vector particle is capable of infecting mammalian cells (e.g, upon reconstitution if they are prepared in a lyophilized form; see e.g. U.S. Ser. No. 08/153,342). Thus, if desired, FIV vector particles can first be either purified and/or concentrated. Representative methods for concentrating and/or purifying vector particles include centrifugation, precipitation (e.g., utilizing PEG), filtration, and column chromatograpy.

FIV vector particles which have been purified or concentrated may be preserved by first adding a sufficient amount of a formulation buffer to the media containing the FIV vector particles, in order to form an aqueous suspension. The formulation buffer is an aqueous solution that contains a saccharide, a high molecular weight structural additive, and a buffering component in water. As utilized within the context of the resent invention, a “buffering compound” or “buffering component” should be understood to refer to a substance that functions to maintain the aqueous suspension at a desired pH. The aqueous solution may also contain one or more amino acids.

The FIV vector particle can also be preserved in a purified form. More specifically, prior to the addition of the formulation buffer, the crude FIV vector particle described above may be clarified by passing it through a filter, and then concentrated, such as by a cross flow concentrating system (Filtron Technology Corp., Nortborough, Mass.). Within one embodiment, DNase is added to the concentrate to digest exogenous DNA. The digest is then diafiltrated to remove excess media components and establish the FIV vector particle in a more desirable buffered solution. The diafiltrate is then passed over a Sephadex S-500 gel column and a purified FIV vector particle is eluted. A sufficient amount of formulation buffer is added to this eluate to reach a desired final concentration of the constituents and to minimally dilute the FIV vector particle, and the aqueous suspension is then stored, preferably at −70° C. or immediately dried. As noted above, the formulation buffer is an aqueous solution that contains a saccharide, a high molecular weight structural additive, and a buffering component in water. The aqueous solution may also contain one or more amino acids.

The crude FIV vector particle can also be purified by ion exchange column chromatography (see U.S. patent application Ser. No. 08/093,436). In general, the crude FIV vector particle is clarified by passing it through a filter, and the filtrate loaded onto a column containing a highly sulfonated cellulose matrix. The FIV vector particle is eluted from the column in purified form by using a high salt buffer. The high salt buffer is then exchanged for a more desirable buffer by passing the eluate over a molecular exclusion column. A sufficient amount of formulation buffer is then added, as discussed above, to the purified FIV vector particle and the aqueous suspension is either dried immediately or stored, preferably at −70° C.

The aqueous suspension in crude or purified form can be dried by lyophilization or evaporation at ambient temperature. Specifically, lyophilization involves the steps of cooling the aqueous suspension below the glass transition temperature or below the eutectic point temperature of the aqueous suspension, and removing water from the cooled suspension by sublimation to form a lyophilized lentivirus. Briefly, aliquots of the formulated FIV vector particle are placed into an Edwards Refrigerated Chamber (3 shelf RC3S unit) attached to a freeze dryer (Supermodulyo 12K). A multistep freeze drying procedure as described by Phillips et al. (Cryobiology 18:414, 1981) is used to lyophilize the formulated FIV vector particle, preferably from a temperature of −40° C. to −45° C. The resulting composition contains less than 10% water by weight of the lyophilized lentivirus. Once lyophilized, the FIV vector particle is stable and may be stored at ″20° C. to 25° C.

Within the evaporative method, water is removed from the aqueous suspension at ambient temperature by evaporation. Within one embodiment, water is removed through spray drying (EP 520,748). Within the spray drying process, the aqueous suspension is delivered into a flow of preheated gas, usually air, whereupon water rapidly evaporates from droplets of the suspension. Spray drying apparatus are available from a number of manufacturers (e.g., Drytec, Ltd., Tonbridge, England; Lab-Plant, Ltd., Huddersfield, England). Once dehydrated, the FIV vector particle is stable and may be stored at −20° C. to 25° C. Within the methods described herein, the resulting moisture content of the dried or lyophilized lentivirus may be determined through use of a Karl-Fischer apparatus (EM Science Aquastar′ V1B volumetric titrator, Cherry Hill, N.J.), or through a gravimetric method.

The aqueous solutions used for formulation, as previously described, are composed of a saccharide, high molecular weight structural additive, a buffering component, and water. The solution may also include one or more amino acids. The combination of these components act to preserve the activity of the FIV vector particle upon freezing and lyophilization, or drying through evaporation. Although a preferred saccharide is lactose, other saccharides may be used, such as sucrose, mannitol, glucose, trehalose, inositol, fructose, maltose or galactose. In addition, combinations of saccharides can be used, for example, lactose and mannitol, or sucrose and mannitol (e.g., a concentration of lactose is 3%-4% by weight. Preferably, the concentration of the saccharide ranges from 1% to 12% by weight.

The high molecular weight structural additive aids in preventing viral aggregation during freezing and provides structural support in the lyophilized or dried state. Within the context of the present invention, structural additives are considered to be of “high molecular weight” if they are greater than 5000 m.w. A preferred high molecular weight structural additive is human serum albumin. However, other substances may also be used, such as hydroxyethyl-cellulose, hydroxymethyl-cellulose, dextran, cellulose, gelatin, or povidone. A particularly preferred concentration of human serum albumin is 0.1% by weight. Preferably, the concentration of the high molecular weight structural additive ranges from 0.1% to 10% by weight.

The amino acids, if present, function to further preserve viral infectivity upon cooling and thawing of the aqueous suspension. In addition, amino acids function to further preserve viral infectivity during sublimation of the cooled aqueous suspension and while in the lyophilized state. A preferred amino acid is arginine, but other amino acids such as lysine, ornithine, serine, glycine, glutamine, asparagine, glutamic acid or aspartic acid can also be used. A particularly preferred arginine concentration is 0.1% by weight. Preferably, the amino acid concentration ranges from 0.1% to 10% by weight.

The buffering component acts to buffer the solution by maintaining a relatively constant pH. A variety of buffers may be used, depending on the pH range desired, preferably between 7.0 and 7.8. Suitable buffers include phosphate buffer and citrate buffer. A particularly preferred pH of the FIV vector particle formulation is 7.4, and a preferred buffer is tromethamine.

In addition, it is preferable that the aqueous solution contain a neutral salt which is used to adjust the final formulated FIV vector particle to an appropriate iso-osmotic salt concentration. Suitable neutral salts include sodium chloride, potassium chloride or magnesium chloride. A preferred salt is sodium chloride.

Aqueous solutions containing the desired concentration of the components described above may be prepared as concentrated stock solutions.

One method of preserving FIV vector particles in a lyophilized state for subsequent reconstitution comprises the steps of (a) combining an infectious FIV vector particle with an aqueous solution to form an aqueous suspension, the aqueous suspension including 4% by weight of lactose, 0.1% by weight of human serum albumin, 0.03% or less by weight of NaCI, 0.1% by weight of arginine, and an amount of tromethamine buffer effective to provide a pH of the aqueous suspension of approximately 7.4, thereby stabilizing the infectious FIV vector particle; (b) cooling the suspension to a temperature of from −40° C. to −45° C. to form a frozen suspension; and (c) removing water from the frozen suspension by sublimation to form a lyophilized composition having less than 2% water by weight of the lyophilized composition, the composition being capable of infecting mammalian cells upon reconstitution. It is preferred that the FIV vector particle be replication defective and suitable for administration into humans upon reconstitution.

It will be evident to those skilled in the art given the disclosure provided herein that it may be preferable to utilize certain saccharides within the aqueous solution when the lyophilized lentivirus is intended for storage at room temperature. More specifically, it is preferable to utilize disaccharides, such as lactose or trehalose, particularly for storage at room temperature.

The lyophilized or dehydrated lentiviruses of the subject invention may be reconstituted using a variety of substances, but are preferably reconstituted using water. In certain instances, dilute salt solutions which bring the final formulation to isotonicity may also be used. In addition, it may be advantageous to use aqueous solutions containing components known to enhance the activity of the reconstituted lentivirus. Such components include cytokines, such as IL-2, polycations, such as protamine sulfate, or other components which enhance the transduction efficiency of the reconstituted lentivirus. Lyophilized or dehydrated FIV vector particle may be reconstituted with any convenient volume of water or the reconstituting agents noted above that allow substantial, and preferably total solubilization of the lyophilized or dehydrated sample.

### ADMINISTRATION

As noted above, high titer recombinant FIV-based particles of the present invention may be administered to a wide variety of locations including, for example, into sites such as the cerebral spinal fluid, bone marrow, joints, arterial endothelial cells, rectum, buccal/sublingual, vagina, the lymph system, to an organ selected from the group consisting of lung, liver, spleen, skin, blood and brain, or to a site selected from the group consisting of tumors and interstitial spaces. Within other embodiments, the FIV vector particle may be administered intraocularly, intranasally, sublinually, orally, topically, intravesically, intrathecally, topically, intravenously, intraperitoneally, intracranially, intramuscularly, or subcutaneously. Other representative routes of administration include gastroscopy, ECRP and colonoscopy, which do not require full operating procedures and hospitalization, but may require the presence of medical personnel.

Considerations for administering the compositions of the present invention include the following:

Oral administration is easy and convenient, economical (no sterility required), safe (over dosage can be treated in most cases), and permits controlled release of the active ingredient of the composition (the lentiviral vector particle). Conversely, there may be local irritation such as nausea, vomiting or diarrhea, erratic absorption for poorly soluble drugs, and the FIV vector particle will be subject to “first pass effect” by hepatic metabolism and gastric acid and enzymatic degradation. Further, there can be slow onset of action, efficient plasma levels may not be reached, a patient's cooperation is required, and food can affect absorption. Preferred embodiments of the present invention include the oral administration of FIV vector particles that express genes encoding erythropoietin, insulin, GM-CSF cytokines, various polypeptides or peptide hormones, their agonists or antagonists, where these hormones can be derived from tissues such as the pituitary, hypothalamus, kidney, endothelial cells, liver, pancreas, bone, hemopoetic marrow, and adrenal. Such polypeptides can be used for induction of growth, regression of tissue, suppression of immune responses, apoptosis, gene expression, blocking receptor-ligand interaction, immune responses and can be treatment for certain anemias, diabetes, infections, high blood pressure, abnormal blood chemistry or chemistries (e.g., elevated blood cholesterol, deficiency of blood clotting factors, elevated LDL with lowered HDL), levels of Alzheimer associated amaloid protein, bone erosion/calcium deposition, and controlling levels of various metabolites such as steroid hormones, purines, and pyrimidines. Preferably, the FIV vector particles are first lyophilized, then filled into capsules and administered.

Buccal/sublingual administration is a convenient method of administration that provides rapid onset of action of the active component(s) of the composition, and avoids first pass metabolism. Thus, there is no gastric acid or enzymatic degradation, and the absorption of FIV vector particles is feasible. There is high bioavailability, and virtually immediate cessation of treatment is possible. Conversely, such administration is limited to relatively low dosages (typically about 10-15 mg), and there can be no simultaneous eating, drinking or swallowing. Preferred embodiments of the present invention include the buccal/sublingual administration of FIV vector particles that contain genes encoding self and/or foreign MHC, or immune modulators, for the treatment of oral cancer; the treatment of Sjogren's syndrome via the buccal/sublingual administration of such lentiviral vector particles that contain IgA or IgE antisense genes; and, the treatment of gingivitis and periodontitis via the buccavsublingual administration of IgG or cytokine antisense genes.

Rectal administration provides a negligible first pass metabolism effect (there is a good blood/lymph vessel supply, and absorbed materials drain directly into the inferior vena cava), and the method is suitable of children, patients with emesis, and the unconscious. The method avoids gastric acid and enzymatic degradation, and the ionization of a composition will not change because the rectal fluid has no buffer capacity (pH 6.8; charged compositions absorb best). Conversely, there may be slow, poor or erratic absorption, irritation, degradation by bacterial flora, and there is a small absorption surface (about 0.05m²). Further, lipidophilic and water soluble compounds are preferred for absorption by the rectal mucosa, and absorption enhancers (e.g., salts, EDTA, NSAID) may be necessary. Preferred embodiments of the present invention include the rectal administration of FIV vector particles that contain genes encoding colon cancer antigens, self and/or foreign MHC, or immune modulators.

Nasal administration avoids first pass metabolism, and gastric acid and enzymatic degradation, and is convenient. In a preferred embodiment, nasal administration is useful for FIV vector particle administration wherein the FIV vector particle is capable of expressing a polypeptide with properties as described herein. Conversely, such administration can cause local irritation, and absorption can be dependent upon the state of the nasal mucosa.

Pulmonary administration also avoids first pass metabolism, and gastric acid and enzymatic degradation, and is convenient. Further, pulmonary administration permits localized actions that minimize systemic side effects and the dosage required for effectiveness, and there can be rapid onset of action and self-medication. Conversely, at times only a small portion of the administered composition reaches the brochioli/alveoli, there can be local irritation, and overdosing is possible. Further, patient cooperation and understanding is preferred, and the propellant for dosing may have toxic effects. Preferred embodiments of the present invention include the pulmonary administration of FIV vector particles that express genes encoding IgA or IgE for the treatment of conditions such as asthma, hay fever, allergic alveolitis or fibrosing alveolitis, the CFTR gene for the treatment of cystic fibrosis, and protease and collagenous inhibitors such as a-1-antitrypsin for the treatment of emphysema. Alternatively, many of the same types of polypeptides or peptides listed above for oral administration may be used.

Ophthalmic administration provides local action, and permit prolonged action where the administration is via inserts. Further, avoids first pass metabolism, and gastric acid and enzymatic degradation, and permits self-administration via the use of eye-drops or contact lens-like inserts. Conversely, the administration is not always efficient, because the administration induces tearing. Preferred embodiments of the present invention include the ophthalmic administration of FIV vector particles that express genes encoding IgA or IgE for the treatment of hay fever conjunctivitis or vernal and atomic conjunctivitis; and ophthalmic administration of FIV vector particles that contain genes encoding melanoma specific antigens (such as high molecular weight-melanoma associated antigen), self and/or foreign MHC, or immune modulators.

Transdermal administration permits rapid cessation of treatment and prolonged action leading to good compliance. Further, local treatment is possible, and avoids first pass metabolism, and gastric acid and enzymatic degradation. Conversely, such administration may cause local irritation, is particularly susceptible to tolerance development, and is typically not preferred for highly potent compositions. Preferred embodiments of the present invention include the transdermal administration of FIV vector particles that express genes encoding IgA or IgE for the treatment of conditions such as atopic dermatitis and other skin allergies; and transdermal administration of FIV vector particles encoding genes encoding melanoma specific antigens (such as high molecular weight-melanoma associated antigen), self and/or foreign MHC, or immune modulators.

Vaginal administration provides local treatment and one preferred route for hormonal administration. Further, such administration avoids first pass metabolism, and gastric acid and enzymatic degradation, and is preferred for administration of compositions wherein the FIV vector particles express peptides. Preferred embodiments of the present invention include the vaginal administration of FIV vector particles that express genes encoding self and/or foreign MHC, or immune modulators. Other preferred embodiments include the vaginal administration of genes encoding the components of sperm such as histone, flagellin, etc., to promote the production of sperm-specific antibodies and thereby prevent pregnancy. This effect may be reversed, and/or pregnancy in some women may be enhanced, by delivering FIV vector particles vectors encoding immunoglobulin antisense genes, which genes interfere with the production of sperm-specific antibodies.

Intravesical administration permits local treatment for urogenital problems, avoiding systemic side effects and avoiding first pass metabolism, and gastric acid and enzymatic degradation. Conversely, the method requires urethral catheterization and requires a highly skilled staff. Preferred embodiments of the present invention include intravesical administration of FIV vector particle encoding antitumor genes such as a prodrug activation gene such thymidine kinase or various immunomodulatory molecules such as cytokines.

Endoscopic retrograde cystopancreatography (ERCP) (goes through the mouth; does not require piercing of the skin) takes advantage of extended gastroscopy, and permits selective access to the biliary tract and the pancreatic duct. Conversely, the method requires a highly skilled staff, and is unpleasant for the patient.

Many of the routes of administration described herein (e.g., into the CSF, into bone marrow, into joints, intravenous, intra-arterial, intracranial intramuscular, subcutaneous, into various organs, intra-tumor, into the interstitial spaces, intra-peritoneal, intralymphatic, or into a capillary bed) may be accomplished simply by direct administration using a needle, catheter or related device. In particular, within certain embodiments of the invention, one or more dosages may be administered directly in the indicated manner at dosages greater than or equal to 10³, 10⁴, 10⁵, 10⁶, 10⁷, 10⁸, 10⁹, 10¹⁻ or 10¹¹ cfu.

FIV vector particle may be delivered to the target from outside of the body (as an outpatient procedure) or as a surgical procedure, where the vector is administered as part of a procedure with other purposes, or as a procedure designed expressly to administer the vector. Other routes and methods for administration include the non-parenteral routes disclosed within U.S. application Ser. No. 08/366,788, filed Dec. 30, 1994, as well as administration via multiple sites as disclosed within U.S. application Ser. No. 08/366,784, filed Dec. 30, 1994.

The following examples are offered by way of illustration, and not by way of limitation.

### EXAMPLES

The following examples describe the construction of a three-plasmnid viral vector system based on FIV. The first construct series described are the FIV vector constructs which contain FIV cis-acting sequences and unique cloning sites for the introduction of one or more genes of interest (FIG. 3). FIV vector/reporter gene constructs are FIV vector constructs which may contain marker genes such as the β-galactosidase (β-gal) gene or human placental alkaline phosphatase (PLAP) gene, the expression of which is easily assayed. The second construct series described are the FIV packaging expression cassettes which provide, with the exception of the FIV envelope protein, the structural, enzymatic and regulatory proteins of FIV. The third component in the three-plasmid vector system is the env expression cassette which may express either the FIV envelope protein or a heterologous envelope protein such as the VSV-G envelope protein. Included in the following examples are also methods for vector particle production, transduction of target cells, assays for transgene expression as well as the production of FIV packaging cell lines.

All constructs were generated using standard molecular biology techniques as described in Sambrook et al., Molecular Cloning: A Laboratory Manual, 2nd ed., Cold Spring Harbor Laboratory Press, 1989). Plasmid DNA was transformed and grown in E. coli HB101 cells and isolated by passage over Qiagen mini- or giga-columns according to manufacturer's instructions. Mutations were introduced using the polymerase chain reaction (PCR), dut⁻, ung⁻ mutagenesis (Muta-gene Kit, BioRad Laboratories, Hercules, Calif.; Kunkel, PNAS 82: 488, 1985) or the Quick-Change In Vitro Mutagenesis Kit (Stratagene, San Diego, Calif.) with oligonucleotides synthesized by Operon Technologies Inc. (Alameda, Calif.). All plasmids were screened by restriction enzyme digestion and their nucleotide sequence confirmed by sequence analysis (Seqwright, LLC, Houston, Tex.).

### Example 1

### CONSTRUCTION OF FIV VECTORS

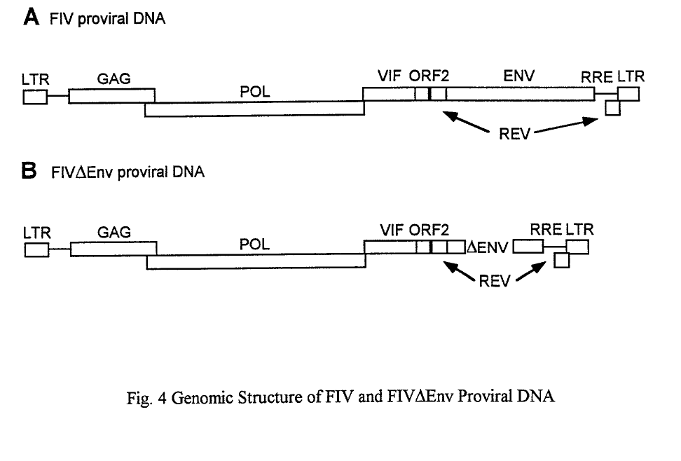

FIV vector, or pTFIV, constructs were generated in a series of steps from FIV-34TF10 (FIV proviral DNA; FIG. 4) which will henceforth be referred to as pF34. pF34 was obtained from NIH AIDS Research and Reference Reagent Program (FIV-34TF10, Cat. No. 1236; Talbott et al., PNAS 86: 5743, 1989) and contains a 9.5 kb (9472 bp) insert from FIV-Petaluma plus 5′ and 3′ flanking cellular DNA cloned into pUC1 19 (SEQ ID N. 1). pTFIV constructs consist of the 5′ and 3′ FIV LTRs from pF34 and some portion of the non-coding region immediately following the 5′ FIV LTR. This portion of the non-coding region includes the first splice donor site and likely includes some part of the putative FIV packaging signal. In addition, pTFIV constructs may contain some portion of the FIV Gag coding region as well as the FIV RRE. The term ‘pTFIV construct’ encompasses two series of constructs, the pTFIVS series and pTFIVL series, which differ by containing either a short (S) or long (L) segment corresponding to the Gag coding region.

A. Construction of the pTFIVS vector

In general, to construct the pTFIVS vector, DNA corresponding to the 5′ FIV LTR plus a portion of the Gag ORF was amplified from pF34 by PCR and cloned into an intermediate plasmid. Likewise, DNA corresponding to the 3′ FIV LTR plus the FIV RRE was amplified from pF34 by PCR and also cloned into an intermediate plasmid. The 5′ FIV LTR fragment was then released from the intermediate construct and ligated into the 3′ FIV LTR-containing intermediate plasmid to create the pTFIVS vector. More specifically, to generate the 5′ region of pTFIVS, FIV primers FIV13 (SEQ ID No. 5) and FIV14 (SEQ ID No. 6) were used to PCR-amplify a fragment corresponding to the 5′ LTR and a 0.35 kb portion of the Gag coding region. FIV13 (SEQ ID No. 5; TTC ATA CCG CGG TGG GAT GAG TAC TGG AAC C) corresponds to the 5′ FIV LTR from nt 1 through nt 31 and contains a Sac II site (underlined) near its 5′ end. FIV 14 (SEQ ID No. 6; CAA ATA GCG GCC GCA GCA GCA GTA GAC ACC) is complementary to a region of the Gag ORF which includes the Tthl 11 I site at nt 920 and contains an additional Not I site (underlined) near its 5′ end. To generate the 3′ region of pTFIVS, primers FIV16 (SEQ ID No. 7) and FIV18 (SEQ ID No. 8) were used to amplify a fragment corresponding to the 3′ FIV LTR and adjacent RRE. FIV16 (SEQ ID No. 7; GTT AAC GGG CCC AAG AAA TAC AAC CAC AAA TGG) corresponds to FIV nt 8761 through 8781 and contains an Apa I site (underlined) near its 5′ terminus. FIV 18 (SEQ ID No. 8; ATC GAT GGT ACC TGC GAA GTT CTC GGC CC) corresponds to the FIV 3′ LTR from nt 9443 to nt 9472 and includes a Kpn I site near its 5′ terminus. PCR samples contained 100 pmol of each primer, 200 W each dNTP, 2 U Pfu DNA polymerase (Stratagene, San Diego, Calif.), 10 μl 10X Pfu buffer and 50 ng pF34 DNA as template. PCR samples were denatured at 95° C. for 2 min then subjected to 25 cycles of denaturation, annealing and extension conditions consisting of 95° C. for 2 min, 55° C. for 0.5 min and 72° C. for 1 min or longer (i.e., 30 sec for each 400 bases to be amplified), respectively. After 25 cycles, reactions were held at 72° C. for 10 min to favor complete extension and then kept at 4° C. for 5 min to overnight. PCR products were gel-purified and ligated directly into pPCR-Script SK (+) (Stratagene, San Diego, Calif.) to generate pCR13/14 and pCR16/17. pCR16/17 was digested with Kpn I and Apa I and the liberated fragment ligated into similarly digested pBlueScript KS II (+) to create pB3′ FIV. pCR13/14 was digested with Sac II and Not I and the resulting fragment ligated into similarly digested pB3′ FIV to create pTFWS.

B. Construction of the pTFIVL vector

The pTFIVL vector was constructed in a manner similar to that of the pTFIVS vector; i.e., the 5′ LTR and 3′ LTR portions were individually amplified by PCR, cloned into intermediate plasmids, then combined to form the complete pTFIVL vector. The 3′ region of pTFIVL is identical to that of pTFIVS and was generated as described in example 1A. The 5′ region of pTFIVL was generated using FIV primers FIV13 (SEQ ID No. 5; Example IA) and FIV15 (SEQ ID No. 9) to amplify a fragment corresponding to the 5′ LTR plus a 0.55 kb portion of the Gag coding region. FIV15 (SEQ ID No. 9; CAA ATA GCG GCC GCG TTG AAC TTC CTC ACC TCC) is complementary to a region of the Gag ORF from nt 1107 to nt 1140 and contains an additional Not I site (underlined) near its 5′ terminus. PCR products were gel-purified and ligated directly into pPCR-Script SK (+) to generate pCR13/15 and pCR16/17 (Example 1A). pCR13/15 was digested with Sac II and Not I and the resulting fragment ligated into similarly digested pB3′ FIV (Example 1A) to create pTFIVL.

### Example 2

### CONSTRUCTION OF HYBRID FIV LTR VECTORS

Hybrid FIV LTR vectors are similar to the FIV vectors described in example 1, however the hybrid vectors contain heterologous enhancer and/or promoter elements in place of all or part of the U3 region of the 5′ FIV proviral DNA LTR. The pTC/FIV constructs, described below, are similar to the pTFIV series but contain CMV promoter or promoter/enhancer elements in place of the FIV U3 region. pTC/FIVS is analogous to pTFIVS with respect to containing a short portion of the Gag coding region while pTC/FIVL is analogous to pTFIVL in containing a long portion of the Gag coding region downstream of the 5′ FIV LTR.

A. Construction of the pTC/FIVS hybrid FIV LTR vector pTC/FIVS, in which the FIV U3 region has been replaced by the CMV promoter/enhancer, was generated using the “splice overlap extension (SOE) PCR” method of Deminie and Emerman (J. Virol. 67: 6499, 1993). Briefly, this method consists of two rounds of PCR, the first-round generating two or three PCR fragments with overlapping regions which are subsequently annealed to one another to serve as template DNA for the second round PCR. For first round PCR, primers FIV19 (SEQ ID No. 10) and FWV20 (SEQ ID No. 11) were used to amplify the region corresponding to the CMV promoter/enhancer from pCMVβ (Clontech Laboratories Inc., Palo Alto, Calif.). In a separate reaction, primers FIV21 (SEQ ID No. 12) and FIV14 (SEQ ID No 6; see Example 1A) were used to generate the FIV U3 and R region from pF34 template DNA (see PCR conditions in Example 1A). FIV19 (SEQ ID No. 10; CCG CGG GAG CTT GCA TGC CTG CAG) corresponds to the CMV enhancer region of pCMVW from nt 1 to nt 24 and but contains a Sac II site (underlined) in place of the EcoR I site at nt 1. The 5′ end of FIV20 (SEQ ID No. 11; TTT CAC AAA GCA CTG GTT ATA TAG ACC TCC CAC CG) is complementary to a region of the CMV promoter up to and including the TATA box (underlined) and the 3′ end is complementary to the FIV R region (italicized). The 5′ end of FIV21 (SEQ ID No.12; CGG TGG GAG GTC TAT ATA ACC AGT GCT TTG TGA AA) corresponds to the CMV promoter and TATA box (underlined) and the 3′ end corresponds the FIV R region (italicized), thus FIV 21 (SEQ ID No. 12) is complementary to FIV20 (SEQ ID No. 11). FIV14 (SEQ ID No. 6) has been described previously (see example 1A). For second round PCR, the FIV 19/20 and FIV 21/14 PCR fragments were gel-purified and 5 Ill of each used as template DNA for the amplification of a CMV/FIV hybrid LTR using FIV19 and FIV14 as primers. The second round PCR product was ligated directly into pPCR-Script SK(+) (Stratagene, San Diego, Calif.) to create pCR19/14. pCR19/14 was then digested with Sac II and Not I and the resulting 1.3 kb fragment ligated into similarly digested pB3′ FIV to create pTC/FIVS.

B. Construction of the pTC/FIVL hybrid FIV LTR vector

The pTC/FIVL hybrid vector is identical to pTC/FIVS except that pTC/FIVL contains a long portion of the Gag coding region downstream of the 5′ FIV LTR. pTC/FIVL was constructed in parallel with pTC/FIVS using the methods described in example 2A. Briefly, to create pTC/FIVL, primer FIV 15 (SEQ ID No. 9; Example 1B) was used in place of primer FIV14 to generate the FIV U3 and R region from pF34 template DNA during first round PCR. For second round PCR, the resulting FIV 21/15 fragment was gel-purified and used together with the FIV 19/20 fragment (Example 2B) and primers FIV 19 (SEQ ID No. 10) and 15 (SEQ ID No. 9) to amplify the CMV/FIV hybrid LTR. The resulting second round PCR product was ligated directly into pPCR-Script SK(+) to create pCR19/15 was then digested with Sac II and Not I and the resulting 1.5 kb fragment ligated into similarly digested pB3′ FIV to create pTC/FIVL.

### Example 3

### INSERTION OF PROMOTER/REPORTER GENE CASSETTES INTO FIV VECTORS

Promoter/reporter gene cassettes consist of a heterologous promoter (e.g., the CMV or SV40 promoter) followed by a reporter gene such as the β-galactosidase (β-gal) gene or human placental alkaline phospatase gene (PLAP). Such cassettes were generated and inserted into one or more FIV vectors or hybrid FIV LTR vectors to create FIV/reporter gene vectors (FIG. 3). FIV/reporter gene vectors may contain the FIV RRE and, in addition, may contain heterologous export elements (HEEs) such as the MPMV CTE or HBV PRE (see detailed description). FIV/reporter gene vectors (e.g., pTFSCβCTE) are named according to the vector backbone (e.g. pTFIVS, in this case shortened as pTFS), the heterologous promoter (e.g., CMV, denoted by C), the reporter gene(s) within the cassette (e.g, β-gal or β) and the heterologous export element (e.g., CTE), if present.

A. Generation of the pCMVD3zal expression cassette

To generate pCMVβgal, a 0.75 kb fragment containing the hCMV (henceforth referred to as CMV) early gene promoter was first liberated from pCMV-G (Yee et al., PNAS 91:9564, 1994) by digestion with Xba I and Sal I. Next, a 3.1 kb Sal I/Sma I fragment containing the β-gal gene was released from pUCβgal. pUCβgal contains the Xba I/SacI and SacI/SmaI β-gal gene fragments from pSP6-β-GAL (Xu et al., Virology 171:331, 1989) cloned into Xba/ISma I digested pUC19 (Clontech Laboratories, Inc. Palo Alto, Calif.). Finally, the 0.75 kb CMV promoter fragment from pCMV-G and the 3.1 kb β-gal gene fragment from pUCβgal were gel-purified, ligated together and inserted into Xba VISma I digested pBluescript SK (−) to create pCMVβgal.

B. Generation of the pCMVβgaICTE expression cassette

The construction of pCMVβgalCTE was accomplished after amplification of the CTE by PCR from MPMV using the oligos CTEH5 (SEQ ID No. 13; GTC AAG CTT AGA CTG GAC AGC CAA TG) and CTEH3 (SEQ ID No. 14; CTA AAG CTT CCA AGA CAT CAT CCG GG), which harbor Hind III sites near their 5′ ends (underlined). The PCR product was digested with Hind III and inserted into the Hind III site of pBluescript SK (−) to create pSK-CTE (personal communication, Dr. Shin-Tai Chen). pSK-CTE was then digested with Sma I and Xho I and the resulting 0.2 kb fragment ligated into similarly digested pCMVβgal (Example 3A) to create pCMVβgalCTE.

C. Generation of the pCMVβgalPRE expression cassette

To generate pCMVβgalPRE, a 0.65 kb fragment was released from pCCAT-1 (Yee, J-K. Science 246: 658, 1989) by digestion with Stu I and Hind III. The 0.65 kb fragment was treated with the Klenow fragment of DNA Polymerase I and ligated into the EcoRV site of pBluescript SK (−) to create pSK-PRE. pSK-PRE was then digested with Sma I and Xho I and the resulting 0.66 kb fragment ligated into similarly digested pCMVβgal (Example 3A) to create pCMVβgalPRE.

D. Generation of the pCMVβgalRRE expression cassette

pCMVβgalRRE was generated in a manner similar to that described for pCMVβgalCTE (Example 3B). The HIV-1 RRE was amplified by PCR from the molecular clone pNL4-3 (Adachi et al., J. Virol. 59: 284, 1986) using the oligos RRE1 (SEQ ID No. 15; GCA AGC TTC TGC AGA GCA GTG GGA ATA GG) and RRE2 (SEQ ID No. 16; GCA AGC TTA CCC CAA ATC CCC AGG AGC TG) which harbor Hind III sites near their 5′ ends (underlined). The amplified product was digested with Hind III and inserted into the Hind III site of pBluescript SK (−) to create pSK-RRE. pSK-RRE was then digested with Sma I and Xho I and the resulting fragment ligated into similarly digested pCMVβgal (Example 3A) to create pCMVβgalRRE.

E. Construction of the pTFSCβ FIV vector

pCMVβgalCTE (Example 3B), containing the CMV promoter/enhancer, β-gal gene and CTE element was the source of reporter gene expression cassette for the construction of the pTFSβ vector. To create pTFSβ, pCMVβgalCTE was digested with Not I and Sma I and the resulting 3.8 kb fragment (containing the CMV promoter and β-gal gene) gel-purified and ligated into similarly digested pTFIVS (Example 1A).

F. Construction of the pTFLCD FIV vector

To create pTFLCβ, pCMVβgalCTE (Example 3B) was digested with Not I and Sma I and the resulting 3.8 kb fragment gel-purified and ligated into similarly digested pTFIVL (Example 1B).

G. Construction of the pTFSCβCTE FIV vector

To create pTFSCβCTE, pCMVβgalCTE (Example 3B), was digested with Not I and Xho I and the resulting 4.0 kb fragment (containing the CMV promoter, β-gal gene and CTE element) gel-purified and ligated into Not I/Sal I digested pTFIVS (Example 1A).

H. Construction of the pTFLCDCTE FIV vector

To create pTFLCβCTE, pCMVβgalCTE (Example 3B), was digested with Not I and Xho I and the resulting 4.0 kb fragment (containing the CMV promoter, β-gal gene and CTE element) gel-purified and ligated into Not I/Sal I digested pTFIVL (Example 1B).

I. Construction of the pTFSCDPRE FIV vector

To create pTFSCβPRE, pCMVβgalPRE (Example 3C) was the source of reporter gene expression cassette. pCMVβgalPRE was digested with Not I and Xho I and the resulting 4.5 kb fragment (containing the CMV promoter, β-gal gene and PRE element) gel-purified and ligated into Not I/Sal I digested pTFIVS (Example 1A).

J. Construction of the pTFLCβPRE FIV vector

To create pTFLCβPRE, pCMVβgalPRE (Example 3C) was digested with Not I and Xho I and the resulting 4.5 kb fragment (containing the CMV promoter, β-gal gene and PRE element) gel-purified and ligated into Not I/Sal I digested pTFIVL (Example 1B).

K. Construction of the pTFSCβRRE FIV vector

To create pTFSCβRRE, pCMVβgalRRE (Example 3D) was the source of reporter gene expression cassette. pCMVβgalRRE was digested with Not I and Xho I and the resulting 4.3 kb fragment (containing the CMV promoter, β-gal gene and RRE element) gel-purified and ligated into Not I/Sal I digested pTFIVS (Example 1A).

L. Construction of the pTFLCDRRE FIV vector

To create pTFLC⊕RRE, pCMVβgalRRE (Example 3D) was digested with Not I and Xho I and the resulting 4.3 kb fragment (containing the CMV promoter, β-gal gene and RRE element) gel-purified and ligated into Not I/Sal I digested pTFIVL (Example 1B).

M. Construction of the pTC/FSCD hybrid FIV LTR vector (pVET_{S}β)

To create pTC/FSCβ, pCMVβgalCTE (Example 3B) was digested with Not I and Sma I and the resulting 3.8 kb fragment (containing the CMV promoter and β-gal gene) gel-purified and ligated into similarly digested pTC/FIVS (Example 2A).

N. Construction of the pTC/FLCβ hybrid FIV LTR vector (pVET_{L}Cβ)

To create pTC/FLCβ, pCMVβgalCTE (Example 3B) was digested with Not I and Sma I and the resulting 3.8 kb fragment (containing the CMV promoter and β-gal gene) gel-purified and ligated into similarly digested pTC/FIVL (Example 2B).

O. Construction of the pTC/FLCβCTE hybrid FIV LTR vector (pVET_{L}CβCTE)

To create pTC/FLCβCTE, pCMVβgalCTE (Example 3B) was digested with Not I and Xho I and the resulting 4.0 kb fragment (containing the CMV promoter, b-gal gene and CTE element) gel-purified and ligated into similarly digested pTC/FIVL (Example 2B).

P. Construction of the pTC/FLCLuc hybrid FIV LTR vector (PVET_{L}CLuc)

The pTC/FLCLuc hybrid FIV LTR vector contains a CMV promoter driving expression of a luciferase (luc) gene. To create pTC/FLCLuc, pCMVLux was used as the source of expression cassette. PCMVLux was derived from pJD204 (De Wet et al., Mol. Cell Biol. 7:725,1987) by liberation of the luciferase encoding fragment (Lux) by Hind III and Bam HI digestion and insertion of the fragment downstream of a CMV promoter. pCMVLux was then digested with Not I and Bam HI and the resulting 2.5 kb fragment gel-purified and ligated into similarly digested pTC/FIVL (Example 2B).

Q. Construction of the pTC/FLCeGFP hybrid FIV LTR vector (PVET_{L}CeGFP)

The pTC/FLCeGFP hybrid FIV LTR vector contains a CMV promoter driving expression of the enhanced green fluorescence protein (eGFP) gene. To create pTC/FLCeGFP, pV653CMVeGFP (J. Glynn, personal communication) was used as the source of expression cassette. pV653CMVeGFP was digested with Xba I and Xho I and the resulting 1.3 kb fragment gel-purified and ligated into an Xba I/Sal I digested pTC/FIVL vector (Example 2B).

R. Construction of the pTC/FLMeGFP hybrid FIV LTR vector (PVET_{L}MeGFP)

The pTC/FLMeGFP hybrid FIV LTR vector contains a MoMLV-derived LTR promoter driving expression of the enhanced green fluorescence protein (EGFP) gene. To create pTCIFLMeGFP, pLTR-eGFP (M. Gasmi, personal communication) was used as the source of expression cassette. pLTR-eGFP was created by first digesting pUCLTR (J.K. Yee, personal communication) with EcoR I and Sma I to release a MoMLV LTR fragment. The eGFP coding fragment was obtained from pEGFP-C (Clontech Laboratories, Inc., Palo Alto, Calif.) by digestion with Eco47 III and Xho I. The MoMLV LTR fragment and eGFP coding fragments were then ligated together with EcoR I/Xho I digested pBluescript SK(−) (Stratagene, San Diego, Calif.) to obtain pLTR-eGFP. Finally, pLTR-eGFP was digested with Nhe I and Xho I and the MoMLV LTR-eGFP cassette ligated into Xba I/Sal I digested pTC/FIVL (Example 2B).

S. Construction of FIV vectors containing an RRE upstream of the promoter/reporter gene cassette

FIV vector constructs in which the FIV RRE was repositioned and, in some cases, replaced with that of HIV-I were generated in a sequence from pTC/FLCβ (Example 3N). The FIV 3′ LTR was amplified by PCR from pTC/FLCβ or pTC/FLCβCTE (Example 30) using primers FIV LTR (SEQ ID No. 17; AAT CAT GGG CCC GGA GAC AGC ACA GTA GAT TC; Apa I site underlined) and FIV 18 (SEQ ID No. 8). pTC/FLCβ or pTC/FLCβCTE was then digested with Apa I and Kpn I and the original 3′ LTR fragments replaced with the similarly digested PCR product to create pTC/FLCβΔRRE and pTC/FLCβCTE .RE To insert RRE elements upstream of the internal CMV promoter, RREs from either FIV-34TF10 or pNL4-3 were first amplified by PCR using the FIV primers FRRE(+) (SEQ ID No. 18; AAT CAT GAC GGT GTC ACC GGT GAA ATT GTA TCC ACA AGA TAC) and FRRE(−) (SEQ ID No. 19; TTA GTT GCG GCC GCT TAC AAT ACA TAC TTT ATT AGT TTG) or HIV primers HRRE(+) (SEQ ID No. 20; AAA TAT GAC GGT GTC ACC GGT AGA GCA GTG GGA ATA GGA G) and HRE(−) (SEQ ID No. 21; CTA AAT GCG GCC GCC CCA AAT CCC CAG GAG C). The resulting PCR products were digested with Tthl 11 I and Not I and inserted into similarly digested pTC/FLCβΔRRE and pTC/FLCβCTEARRE to create pTC/FSCβFR, pTCFSCβHR, pTC/FSCβFRC and pTC/FSCβHRC with each construct containing either the FIV or HIV-1 RRE upstream of the internal CMV promoter and with the latter two constructs containing an additional CTE element downstream of the internal cassette.

T. Construction of the pTC/FLRβgluc hybrid FIV LTR vector (PVET_{L}Rβgluc)

The pTC/FLRβgluc hybrid FIV LTR vector contains an RSV promoter driving the expression of the mammalian β-glucuronidase (β-gluc) gene. To construct pTC/FLRβgluc, an RSV promoter lacking a functional polyadenylation signal was first liberated from pUC19RSV (J. K. Yee, personal communication) by digestion with BarnH I and Sal I. The resulting 320 bp fragment containing the RSV promoter was then inserted into similarly digested pTC/FIVL (Example 2B) to generate pTC/FLRSV. Next, the β-gluc gene was liberated from pAdRSV4 (B. Davidson, personal communication) by digestion with Xho I and the resulting 2.2 kb fragment ligated into Sal I digested/CIP treated pTC/FLRSV to generate pTC/FLR,gluc (⁺ polyA). To remove the polyadenylation signal from the β-gluc cDNA, a portion of the β-gluc gene was amplified by PCR using primers GLUC2 (SEQ ID NO. 22; AAT CCT AGG CTC GAG GAA GGG ACA CGC AGG TGG) and GLUC3 (SEQ ID NO. 23; CGT GGA GCA AGA CAG TGG GC). The PCR product was then digested with Bgl II and Xho I and the resulting 1.2 kb fragment joined in a three-way ligation with a 2.3 kb Nco I/Bgl II fragment and a 3.9 kb Xho I/Nco I fragment from pTC/FLRβgluc (⁺ polyA) to generate pTC/FLRβgluc (−polyA).

U. Construction of the pTC/FLMCFTR hybrid FIV LTR vector (PVET_{L}MCFTR)

The pTC/FLMCFTR hybrid FIV LTR vector contains an MLV promoter driving the expression of the cystic fibrosis transmembrane conductance regulator (CFTR; CFTR on plasmid pBQ4.7, Johanna M. Rommens, University of Toronto, Canada) gene. To generate pTC/FLMCFTR, the MLV promoter was liberated from pUCLTR (J. K. Yee, personal communication) by digestion with Nhe I and Xma I and the resulting 0.5 kb fragment gel-purified. Next, the CFTR gene was liberated from pBA-9b/CFTR (M. Bodner, personal communication) using Xma I and Eco RV and the resulting 2.9 kb fragment gel-purified. pBA-9b/CFTR is a MLV-derived retroviral vector with the CFTR gene cloned as a Not I to Cla I fragment into Not I/Cla I digested pBA-9b (Example 3V). Lastly, the pTC/FIVL (Example 2B) FIV vector was digested with Spe I and Eco RV, gel-purified and ligated together with the above 0.5 and 2.9 kb fragments to create pTC/FLMCFTR.

V. Construction of the pTC/FLMEpo hvbrid FIV LTR vector (pVET_{L}MEpo)

The pTC/FLMEpo hybrid FIV LTR vector has the internal MoMLV LTR promoter and contains the cDNA for erythropoietin (epo), isolated from the kidney of Macaca fascicularis (Cynomolgus monkey). The epo gene was first cloned from the plasmid pMKE83 (ATCC #67545, includes 1.6 Kb of the epo cDNA) into the MLV-derived retroviral vector pBA-6b/L1. pBA-6b/L1 is a derivative of pBA-6b (see PCT publication number WO 97/42338 entitled “Crossless Retroviral Vectors”) with an additional LI linker in the multiple cloning site. Briefly, the 1.5 kb EcoR I to Hind III fragment from pMKB83 including the epo cDNA was ligated into EcoR I and Hind III digested pBluescript KS(+) to create pKS-Epo. pKS-Epo was linearized with EcoR I, blunted and ligated in the presence of Xho I linker DNA (12-mer; SEQ ID No. 24; CCGCTCGAGCGG, New England Biolabs, Inc., Beverly, MA) resulting in pKS-Epo(R/X). pKS-Epo(R/X) was digested with Xho I and Cla I yielding a 1.5 kb epo cDNA fragment. This fragment was ligated into Xho FCla I digested pBA-6b/L1 resulting in plasmid pBA-6b/L1-Epo.

For the construction of pTC/FLMEpo, the plasmids pBA-9b and pFIV-LTR were used. pBA-9b is based on pBA-5b (see PCT publication number WO 97/42338 entitled “Crossless Retroviral Vectors”) with additional restriction enzyme sites in the multiple cloning site. pFIV-LTR was created by digest of pUCLTR (J. K. Yee, personal communication) with Nhe I and Sma I. The resulting 0.6 kb MoMLV LTR fragment was ligated into Xba I/Xma I digested pTC/FIVL (Example 2B) to yield pFIV-LTR.

Briefly, the 1.5 kb epo cDNA from pBA-6b/L1-Epo was liberated by xho I/Cla I digest and the fragment cloned into Xho I/Cla I digested MLV-based retroviral vector pBA-9b to yield pBA-9b/Epo. pBA-9b/Epo was digested with EcoR I and Apa I and the 1.0 kb Epo cDNA fragment ligated into EcoR V/Apa I digested pFIV-LTR to yield pTC/FLMEpo. The epo cDNA in pTC/FLMEpo includes 252 nt of the 5′ untranslated region and 217 nt of the 3′ untranslated region.

W. Construction of the pTC/FLMEpo-short hybrid FIV LTR vector (pVET_{L}MEpo(Δ5′))

In order to delete most of the 5′ untranslated region of the epo cDNA in pTC/FLMEpo, pTC/FLMEpo was digested with EcoR I and Avr II, blunted and religated to yield pTC/FLMEpo-short. This plasmid has 35 nt of the 5′ untranslated region and 217 nt of the 3′ untranslated region of the Epo cDNA.

X. Construction of the pTC/IFLCEpo hybrid FIV LTR vector (pVET_{L}CEpo)

The pTC/FLCEpo hybrid FIV LTR vector has the internal CMV promoter and contains the cDNA for erythropoietin (epo). The plasmid pTC/FIVL (Example 2B) was digested with Apa I and Not I, dephosphorylated with CIP (calf intestinal phosphatase) and gel-purified. The epo cDNA was isolated from pBA-6b/L1-Epo (Example 3V) by Avr II and Apa I digestion and the resulting fragment gel-purified. The source of the CMV promoter was a PCR fragment using pCMVO as a template. The forward primer CMV1 contains a Not I restriction enzyme site (SEQ ID No. 25; AGA TCT GCG GCC GCG AGC TTG CAT GCC TGC AG; Not I site underlined) and the reverse primer CMV2 contains an Xba I restriction enzyme site (SEQ ID No. 26; CTC GAG TAC CGG ATC CTC TAG A; Xba I site underlined). The resulting PCR fragment was cloned into pZero-blunt (Invitrogen, Carlsbad, Calif.) to yield pZero-CMV. pZero-CMV was digested with Not I and Xba I and the Not I-Xba I fragment gel-purified. A three-way ligation with the CMV promoter (Not I-Xba I fragment), the Epo cDNA (Avr II-Apa I fragment) and the Apa I-Not I digested pTC/FIVL vector backbone (Example 2B) resulted in the final vector pTC/FLCEpo. The epo cDNA in pTC/FLCEpo contains 3 5 nt of 5′ untranslated region and 217 nt of 3′ untranslated region.

Y. Construction of the pTC/FLPEpo hybrid FIV LTR vector (pVET_{L}PEpo)

The pTC/FLPEpo hybrid FIV LTR vector has the internal PGK promoter and contains the cDNA for erythropoietin (epo). Plasmid pTC/FIVL (Example 2B) was digested with EcoR I and Apa I, dephosphorylated with CIP (calf intestinal phosphatase) and gel-purified. The epo cDNA was isolated from pBA-6b/L1-Epo (Example 3V) by EcoR I and Apa I digestion. The EcoR I-Apa I fragment with the epo cDNA was gel-purified and ligated into EcoR I/Apa I digested pTC/FIVL, resulting in pTC/FLEpo(252/217). To insert the PGK promoter upstream of the epo cDNA, the PGK promoter was synthesized by PCR using the plasrnid pBSU (Singer-Sam et al., Gene 32:409-417, 1984) for the template. The forward primer codes for a BamH I site (SEQ ID NO.27; CGG GAT CCG GGG TTG GGG TTG CGC CTT TT; BaniH I site underlined) and the reverse primer codes for an EcoR I site (SEQ ID NO.28; CGG AAT TCT CTA GAT TTG GAA ATA CAG CTG GGG AG; EcoR I site underlined). The PCR product was cloned into the into pZero-blunt (Invitrogen, Carlsbad, Calif.) to yield pZero-PGK. The PGK promoter was removed by digesting with BamiH I and EcoR I, the fragment gel-purified and ligated into BamH I/EcoR I digested pTC/FLEpo(252/217) resulting in the final vector pTC/FLPEpo. The epo cDNA contains 252 nt of 5′ untranslated region and 217 nt of 3′ untranslated region.

Z. Construction of the pTC/FLC16K-Ser hybrid FIV LTR vector (pVET_{L}C16K-Ser)

The pTC/FLC16K-Ser hybrid FIV LTR vector has the internal CMV promoter driving the cDNA for 16K. The 16K gene is the N-terminal fragment of prolactin and acts as an anti-angiogenesis factor (Clapp et al., Endocrinology 133:1292, 1993; Ferrara et al., Endocrinology 129:896, 1991). The 16K gene with cysteine at position 58 (16K-Cys) cannot be secreted from mammalian cells but the 16K gene with serine at position 58 (16K-Ser) has been shown to be secreted from mammalian cells (personal communication with Dr. Richard Weiner's lab, UCSF, Calif.). First, the MLV-based retroviral vector pBA-9b/C16K-Cys was produced as follows. The 16K-Cys gene was synthesized by PCR using plasmid pRC/CMV16KChPRL (S140) as a template. The forward primer 16KXbaF introduced an Xba I site (SEQ ID NO. 29; CTA GTC TAG ACA CCA TGA ACA TCA AAG G; Xba I site underlined) and the reverse primer 16KApaR introduced an Apa I site (SEQ ID NO. 30; ATT ACA GGG CCC TCA AGG ATG AAC CTG GCT GAC; Apa I site underlined). The MLV-based retroviral backbone pBA-9b (Example 3V) was digested with Not I and BamH I. The CMV promoter was isolated by Not I and Xba I digestion of pZero-CMV (Example 3X). A three-way ligation with the CMV promoter (Not I-Xba I fragment), the 16K-Cys PCR product (Xba I-BamH I fragment) and Not I/BamlH I digested pBA-9b was carried out resulting in pBA-9b/C-16K-Cys.

To produce pTC/FLC16K-Cys, pBA-9b/C-16K-Cys was digested with Not I and BamH I which released the CMV-16K-Cys fragment. The CMV-16K-Cys fragment was cloned into Not I/BamH I digested pTC/FIVL (Example 2B) resulting in pTC/FLC16K-Cys. In order to produce the 16K version of the FIV vector that is secreted in mammalian cells the 16K-Cys was replaced with the 16K-Ser as follows. pTC/FLC16K-Cys was digested with BstE II and BamH I and the released 16K-Cys fragment replaced with the BstE II/BamH digested 16K-Ser fragment from plasmid pRC/CMV16KShPRL (S140), resulting in pTC/FLC16K-Ser.

AA. Construction of the pTC/FLM16K-Ser hybrid FIV LTR vector (pVET_{L}M16K-Ser)

The pTC/FLM16K-Ser hybrid FIV LTR vector has the internal MoMLV LTR promoter driving the cDNA for 16K (Example 3Z). The 16K-Ser gene was synthesized by PCR using plasmid pRC/CMV16KShPRL (S 140) as a template. The forward primer 16KXbaF (SEQ ID No. 29; Example 3Z) introduced an Xba I site and the reverse primer 16KclaR introduced a Cla I site (SEQ ID NO. 31; ATT ATT ATC GAT TCA AGG ATG AAC CTG GCT GAC; Cla I site underlined). The PCR product was cloned into pZero-blunt (Invitrogen, Carlsbad, Calif.) to yield pZero-16K-Ser. pZero-16K-Ser was digested with EcoR V and Cla I and the 0.45 kb fragment coding for the 16K-Ser cDNA cloned into Sma I and Cla I digested FIV-LTR (Example 3V) resulting in pTC/FLM16K-Ser.

AB. Construction of the pTC/FLChGH hybrid FIV LTR vector (pVET_{L}ChGH)

The pTC/FLChGH hybrid FIV LTR vector contains a CMV promoter driving the human growth hormone gene. The pTC/FLChGH vector was constructed, in part, by using an HIV-based vector already containing a CMV promoter/human growth hormone gene cassette, pV653MCMVhgH (M. Gasmi, personal communication). The expression cassette was liberated from pV653MCMVhgH by digestion with Not I and Cla I and inserted into similarly digested pTC/FIVL (Example 2B) to create pTC/FLChGH.

### Example 4

### INSERTION OF REPORTER GENE CASSETTES INTO FIV VECTORS

To generate FIV vectors containing heterologous genes but lacking heterologous promoters to drive the transcription of such genes, FIV vectors were generated in which transcription of the heterologous gene (e.g., reporter gene) is driven by the FIV 5′ LTR.

A. Construction of the pTFSO FIV vector

To create pTFSβ, pTFIVS (Example 1A) was digested with Xba I and Sma I and ligated together with the 3.1 kb Xba I/Sma I fragment containing the β-gal gene from pCMVβgal (Example 3A).

B. Construction of the pTFLO FIV vector

To create pTFLβ, pTFIVL (Example 1B) was digested with Xba I and Sma I and ligated together with the 3.1 kb Xba I/Sma I fragment containing the β-gal gene from pCMVβgal (Example 3A).

C. Construction of the pTC/FLEpo hybrid FIV LTR vector ( pVET_{L}Epo)

The pTC/FLEpo hybrid FIV LTR vector has no internal promoter and contains the cDNA for erythropoietin (epo). To create pTC/FLEpo, pBA-6bAL1-Epo (Example 3V) was digested with EcoR I and Cla I to liberate the 1.5 kb long epo cDNA. The EcoR I-Cla I fragment was cloned into EcoRi/Cla I digested pTC/FIVL (Example 2B). The epo cDNA in pTC/FLEpo is 1.5 kb long and contains a stretch of 252 nt in the 5′ untranslated region and 625 nt in the 3′ untranslated region.

### Example 5

### CONSTRUCTION OF FIV PACKAGING EXPRESSION CASSEITES

The FIV packaging expression cassettes (pCMVFIV constructs) contain the FIV gag, pol, vif, rev and ORF 2, flanked by the CMV promoter at the 5′ end and SV40 polyadenylation signal at the 3′ end (FIG. 3). The pCMVFIV packaging constructs were generated in a series of steps beginning with the deletion of a 1.6 kb region corresponding to the FIV env gene in pF34 (Example 1). Briefly, pF34 was digested with Kpn I and Spe I and the 1.9 kb env fragment inserted into similarly digested pBluescript II KS(+) to generate pBF34env. pBF34env was digested with Avr II and Spe I, releasing a 1.6 kb product, and religated to generate pBF34Aenv. pBF34Aenv was then digested with Kpn I and Xba I and the resulting 0.3 kb product gel purified and ligated into Kpn I/Spe I digested pF34 to create pF34Δenv (FIVΔenv provirus; FIG. 4). pF34Δenv was then used as the source of FIV sequences for constructing the following pCMVFIV packaging cassettes.

The pCMVFIV packaging constructs described below, differ by containing various lengths of sequence corresponding to the FIV 5′ noncoding region downstream of the 5′ FIV LTR. pCMVFIVXho was constructed using a convenient Xho I site located at nt 500 and therefore contains 0.1 kb of noncoding sequence upstream of the FIV (SD₆₀₄) 5′ splice donor site (i.e., lacks 0.14 kb of the 0.24 kb noncoding sequence between the 3′ border of the 5′ LTR and the 5′ splice donor site). pCMVFIVSal was created after the introduction of a Sal I site at nt 578 and therefore contains only 0.02 kb of noncoding sequence (i.e., lacks 0.22 kb of the noncoding sequence). The Δ17 mutation in pCMVFIVΔ17 and pCMVFIVSaIΔ17 refers to a deletion of 17 bp in the sequence corresponding to the region between the FIV 5′ splice donor and the ATG codon of gag.

A. Construction of Packaging Expression Cassette, pCMVFIVXho (PCFIVX)

To generate pCMVFIVXho from pF34Δenv, a Not I restriction enzyme recognition site was first introduced into pF34Δenv at nt 9168 by oligonucleotide directed in vitro mutagenesis using two rounds of PCR. The first round PCR contained 200 liM each dNTP, 2 U Pfu DNA polymerase, 10 μl 10X Pfu buffer, 50 ng template DNA (pF34 plasmid DNA and 100 μmol each of primers FIV5 (SEQ ID NO. 32; AAA TGG TAG GCA ATG TGG C) and FIV6 (SEQ ID NO. 33; CCT TTT ATC ATT TGT TCG TAA GCG GCC GCT AGT CCA TAA GCA TTC TTT C) or, in a separate reaction, 100 μmol each of primers FIV7 (SEQ ID No. 34; GAA AGA ATG CTT ATG GAC TAG CGG CCG CTT ACG AAC AAA TGA TAA AAG G) and FIV8 (SEQ ID No. 35; CAC TTT ATG CTT CCG GCT C). PCR samples were denatured at 95° C. for 2 min then subjected to 25 cycles of denaturation, annealing and extension conditions of 95° C. for 2 min, 55° C. for 30 sec and 72° C. for 1 min or longer (i.e., 30s for each 400 bases to be amplified), respectively. After 25 cycles, reactions were held at 72° C. for 10 min to favor complete extension and then kept at 4° C. for 5 min to overnight. The second round PCR was identical to the first but with 5 μl gel each gel-purified PCR product serving as template DNA (either the 0.38 kb FIV 5/6 fragment or the 0.6 kb FIV 7/8 fragment) and oligos FIV5 (SEQ ID No. 32) and FIV8 (SEQ ID No. 35) serving as primers. The 0.95 kb second round PCR product was purified, cleaved with Nde I and Sal I, and the resulting 0.74 kb product ligated into similarly digested pF34Δenv to generate pF34NΔenv. pF34NΔenv was then digested either with Tth 111 I and Not I to obtain a 6.7 kb fragment or Xho I and Tth111 I to generate a 0.4 kb product. The purified 6.7 kb and 0.4 kb products were ligated together with a purified 3.6 kb Not LXho I fragment from pCMVβ to create pCMVFIVXho which will henceforth be referred to as pCFIVX.

B. Construction of Packaging Expression Cassette, pCMVFIVSal

To generate pCMVFIVSal, a Sal I restriction enzyme recognition site was first introduced into pF34NΔenv by in vitro mutagenesis as described above. The first round PCR contained either oligos FIVI (SEQ ID No. 36; TGA GGA AGT GAA GCT AGA GC) and FIV2 (SEQ ID No. 37; GTT GAC TGT CCC TCG GCG AGT CGA CTG GCT TGA AGG TCC GCG) or oligos FIV 3 (SEQ ID No. 38; CGC GGA CCT TCA AGC CAG TCG ACT CGC CGA GGG ACA GTC AAC) and FIV4 (SEQ ID No. 39; TTG AAC TTC CTC ACC TCC TAG) and generated either a 0.2 kb or 0.54 kb PCR product, respectively. The second round PCR, containing the purified first round products and oligos FIVI (SEQ ID No. 36) and FIV4 (SEQ ID No. 39), gave rise to a 0.75 kb product. The purified second round product was digested with Tthl II and Sac I and the resulting 0.4 kb product ligated into similarly digested pF34NΔenv to create pF34NSΔenv. pF34NSΔenv was then cleaved with Sal I and Not I and ligated into Xho I/Notl digested pCMVβto create pCMVFIVSal.

C. Construction of Packaging Expression Cassette. pCMVFIVΔ17S (pCFIVΔORF2)

To generate pCMVFIVΔ17S, in vitro mutagenesis was carried out either using oligos FIV1 (SEQ ID No. 36; Example 5B) and FIV9 (SEQ ID No. 40; CCC CTG TCC ATT CCC CAT CCT ACC TTG TYG ACT GTC CCT CGG CGA A where Y is C or T) or using oligos FIV10 (SEQ ID No. 41; GGA CAG TCR ACA AGG TAG GAT GGG GAA TGG ACA GGG G where R is A or G) and FIV4 (SEQ ID No. 39; Example SB) in the first round PCR. The second round PCR contained the 0.23 kb and 0.53 kb products resulting from first round PCR and oligos FIV1 and FIV4. The 0.73 kb second round PCR product was then digested with Sac I and Tthl 11 I and ligated into similarly digested pF34NΔenv to generate pF34NΔ17SΔenv. As above, this latter product was cleaved with Sal I and Not I and ligated into Xho I/Not I digested pCMVβ to generate pCMVFIVΔ17S which will henceforth be referred to as pCFIVΔORF2.

D. Construction of Packaging Expression Cassette, pCMVFIVSalΔ17

A construct similar to pCMVFIVΔ17S, described above, pCMVFIVSalΔ17, was generated by virtue of oligo FIV9 (SEQ ID No. 9; Example 5C) being a degenerate oligo (which may or may not cause the introduction of a Sal I site during in vitro mutagenesis). By using the degenerate oligo FIV9 as a primer (along with FIV1; SEQ ID No. 36; example 5B) and pF34NSΔenv as the DNA template for first round PCR (as described in example 5C), the Δ17 mutation could be made without the introduction of an adjacent Sal I site. The 0.73 kb second round PCR product was digested with Sac I and Tthl 11 1, as above and the resulting fragment ligated into pF34NΔenv to generate pF34NSΔ17Δenv. This latter product was cleaved with Sal I and Not I, as above and ligated into Xho I/Not I digested pCMVO to generate pCMVFIVSalΔ17.

E. Construction of Packaing Expression Cassette. pCFIV

The packaging expression cassette, pCFIV, was created from pCMVFIVΔ17S (now referred to as pCFIVΔORF2, Example 5C). The ORF2 of pCFIVΔORF2 (which contains a premature stop codon) was replaced with the complete ORF2 from FIV14 (which will henceforth be referred to as pF14; Cat# 851, obtained from the NIH AIDS Research and Reference Reagent Program). pF14 was used as the template in a SOE PCR reaction similar to that described in Example 2A. The primers used for first round PCR were as follows: p14-1(+) (SEQ ID No. 42; ATG TTG GCG TGT GGC GTG) and pl4-2(−) (SEQ ID No. 43; AAT ATA AAT ATT TCT AAG CAG TAG TTA TTG). The primers used for second round PCR were as follows: p34-4(+) (SEQ ID No. 44; CAA TAA CTA CTG CTT AGA AAT ATT TAT ATT) and p34-5(−) (SEQ ID No. 45; TTT CTT TTT CTG TTA TTC CAG G). The second round PCR product was purified by phenol/chloroform extraction followed by ethanol precipitation and digested with Ngo MI and Kpn I. The resulting 0.5 kb fragment was then gel-purified and ligated into similarly digested pCFIVΔORF2 to generate pCFIV.

F. Construction of Packing Expression Cassette pCFIVΔVIF

To create pCFIVΔVIF, pCFIV (Example 5E) was first digested with Eco47 III and the blunt ends dephosphorylated by CIP treatment. The phosphorylated complementary oligos AGE(+) (TAA CCG GT) and AGE(−) (ACC GGT TA) were then inserted into the Eco47 μlI site and pCFIV to create pCFIVΔVIF.

G. Construction of Packaging Expression Cassette, pCFIVΔORF2ΔVIF

To create pCFIVΔORF2ΔVIF, pCFIVΔORF2 (Example 5C) was first digested with Eco47 μlI and the blunt ends dephosphorylated by CIP treatment. The phosphorylated complementary oligos AGE(+) (Example 3F) and AGE(−) (Example 3F) were then inserted into the Eco47 III site of pCFIV to create pCFIVΔORF2ΔVIF.

H. Construction of FIV rev Deletion Packaging Expression Cassettes

FIV packaging constructs which either do not express rev or lack the Rev coding regions were generated from pCFIVΔORF2ΔVIF (Example 5G). To generate pCFIVArev, the splice acceptor site and basic amino acid domain of the second exon of rev was deleted in a manner simnilar to that described in Phillips et al., J. Virol. 66:5464, 1992. A region corresponding to nt 9022 to nt 9168 of FIV 34TF10 was PCR amplified using primers ASA (SEQ ID No. 46; TGA TCA TTC GGA GAT CGC TTC AGG AAG C) and FIV6 (SEQ IID No. 33; Example 5A). The ΔSA/FIV6 PCR fragment was digested with BstBl/Not I and inserted into similarly digested pCFIVΔORF2ΔFIV, resulting in a 100 bp deletion and creating pCFIVΔrev. pCFIVFRRE was created by first generating a fragment corresponding to the FIV RRE by PCR amplification. Primers FRRE⁺ (SEQ ID No. 18; Example 3S) and FRRE- (SEQ ID No. 19; Example 3S) were used to amplify the FIV RRE from nt 8701 to nt 8952 from FIV 34TF10. The resulting PCR fragment was digested with Age I/Not I and inserted into similarly digested pCFIVΔORF2ΔVIF resulting in the simultaneous insertion of FIV RRE and the deletion of all FIV sequences extending past the 5′ end of vif. pCFIVHRRE was created in a similar manner using primers HRRE⁺ (SEQ ID No. 20; Example 3S) and HRRE-(SEQ ID No. 21; Example 3S) to generate a region corresponding to HIV RRE from nt 7744 to 8004 corresponding to NL4-3. As above, the resulting PCR fragment was digested with Age I/Not I and inserted into similarly digested pCFIVΔORF2ΔVIF to create pCFIVHRRE. All constructs were screened by restriction enzyme digestion and the sequence of regions amplified by PCR confirmed by sequence analysis.

I. Construction of Packaging Expression Cassette, pCFIVΔSDFRRE

pCFIVΔSDΔrev is one of several FIV packaging constructs lacking the major FIV splice donor site which was used in the generation an FIV packaging cell line. To generate pCFIVΔSDFRRE, a 1.3 kb fragment corresponding to the 5′ proximal coding region of gag was first amplified by PCR using the primers ΔSD-1(+) (SEQ ID No. 47; ACT GCA GGT CGA CCA TGG GGA ATG GAC AGG GG) and FIV(−) (SEQ ID No. 60; GGG GGA ATT CTT TCT ATT TCC TTG CC). The fragment was then gel-purified and digested with Sal I and EcoR I. The fragment was cloned into the Sal I and EcoR I digested pSK(+), resulting in pSK(+)ΔSDFIVgag. Next, a 3.7 kb fragment was liberated from pCFIVFRRE (Example 5H) by digestion with EcoR I and Not I. This fragment was cloned into the EcoR I and Not I digested pSK(+)ΔSDFIVgag, resulting in pSK(+)ΔSDFIVg/pFRRE. A 3.5 kb fragment was then liberated from pCMV by digestion with Not I and Xho I. The digested product was purified and the 0.5 kb fragment was ligated into the Not I and Xho I digested pSK(+)ΔSDFIVg/pFRRE, resulting in pCFIVΔSDFRRE.

J. Construction of Packaging Expression Cassette, pCFIVΔSDHRRE

pCFIVΔSDFRRE also lacks the major FIV splice donor site and was prepared in a method similar to that described in Example 5I except that pCFIVHRRE (Example 5H) replaced pCFIVΔORF2ΔVIF as the source of the EcoR I/Not I digestion product. Next, a 3.7 kb fragment was liberated from pCFIVHRRE (Example 5H) by digestion with EcoR I and Not I. This fragment was cloned into the EcoR I and Not I digested pSK(+)ΔSDFIVgag, resulting in pSK(+)ΔSDFIVg/pHRRE. A 3.5 kb fragment was then liberated from pCMVO by digestion with Not I and Xho I. The digested product was purified and the 0.5 kb fragment was ligated into the Not I and Xho I digested pSK(+)ΔSDFIVg/pBRRE, resulting in pCFIVΔSDHRRE. As above, the 3.5 kb Not I/Xho fragment from pCMVO was ligated into Not I/Xho digested pSK(+)ΔSDFIVg/pHRRE, resulting in pCFIVΔSDHRRE.

K. Construction of Packaging Expression Cassette, pCFIVΔSD4XCTE

pCFIVΔSD4XCTE also lacks the major FIV splice donor site and was prepared, in part, as described in Example 51. Following the generation of pCFIVΔSDFRRE, the construct was digested with Age I and Not I, blunted and a PCR fragment containing four tandemly repeated copies (4X) of the MPMV CTE element (Bray, M. et al., PNAS 91: 1256, 1994) was inserted. The fragment containing the 4X CTE was amplified by PCR from pcDNΔ3-CCCC (provided by Hans-Georg Kraeusslich, Heinrich-Pette-Institute, Hamburg, Germany) using the forward primer CTE1 (SEQ ID No. 48; CTT AAG ACC GGT GGA TCC ACT AGT TCT AGA CC) and CTE2 (SEQ ID No. 49; GCA TGC TCG AGC GGC CGC T). The resulting PCR product was ligated into the blunted pCFIVΔSDFRRE, resulting in pCFIVΔSD4XCTE. This construct will be isolated with the 4XCTE in both orientations, resulting in pCFIVΔSD4XCTE⁺ and pCFIVΔSD4XCTE-.

L. Construction of Packaging Expression Cassette. pCFIVΔSDg/p

pCFIVΔSDg/p also lacks the major FIV splice donor site and does not contain an export element. pCFIVΔSDHRRE (Example 5J) was digested with Age I and Not I, blunted and religated, resulting in pCFIVΔSDg/p.

### Example 6

PRODUCTION OF PSEUDOTYPED FIV PARTICLES

FIV particles lacking the FIV envelope protein but containing the VSV-G envelope protein (i.e., pseudotyped with VSV-G Env) were produced by cotransfection of the FIV envelope deletion construct, pF34Δenv (Example 5), and a VSV-G envelope-expressing plasmid, pCMV-G (Yee et al., PNAS 91: 9564, 1994) into Crandell feline kidney (CrFK) cells. Calcium phosphate-DNA complexes were prepared using the Profectin kit (Promega Corp. Madison, Wis.) according to the manufacturer's instructions using a 1:1 ratio of pF34Δenv and pCMV-G plasmid DNA. Following transfection, the cells were placed in a 5% CO₂ incubator for 6 hr. to overnight after which the medium was replaced and the cells returned to 10% CO₂ for an additional 36 to 66 hr. (i.e., 48 to 72 hr. following transfection). The supernatant was then harvested, filtered through a 0.45 μM Nalgene filter and either used immediately for infection or frozen at −70 C until further use.

### Example 7

INFECTON OF CULTURED CELLS BY PSEUDOTYPED FIV PARTICLES

Serial dilutions of supernatant containing pseudotyped FIV particles (Example 6) were incubated with CrFK, HT1080, or 293 cells in culture medium containing 8 μg/ml polybrene. After 12 to 24 hr incubation, the culture medium was removed, the cells washed three times with PBS, and then maintained in DMEM supplemented with 10% FBS for an additional 24 to 60 hr (i.e., 48 to 72 hr after initial infection) at 10% CO₂. The supernatant was then removed and assayed for the presence of the FIV major core protein (Gag) using the PetCheck FIV Antigen Test Kit (IDEXX, Portland, Me.) according to manufacturer's instructions. The presence of FIV p24 (referred to by its original designation in the IDEXX kit, however, more recently designated as p24; Talbott et al., PNAS 86:5743, 1989; Tilton et al., J. Clin. Microbiol. 28:898), indicated that pseudotyped FIV particles can be produced by cotransfection in CrFK cells and that these particles are capable of infecting naive CrFK cells. Preliminary results are summarized in Table 1.

TABLE 1
Production of p24 in human (HT-1080) and feline (CrFK) kidney cells
after transduction with pF34Δenv

| | HT1080 | CRFK |
|---|---|---|
| pF34 (FIV env^{a}) | −^{b} | + |
| pF34Δenv | − | − |
| pF34Δenv | + | +++ |
| (VSV-G^{a}) | | |

^{a}refers to the virus particle envelope protein
^{b}transduction assessed from FIV p24 levels

### Example 8

### PRODUCTION OF FIV VECTOR PARTICLES

FIV vector particles were produced by transient triple transfection of an FIV/reporter gene vector, an FIV packaging expression construct and a VSV-G envelope-expressing plasmid into 293T human kidney cells. DNA complexes were prepared using calcium phosphate (e.g., Profectin kit; Promega Corp., Madison, Wis.) and transfected into cells according to the manufacturer's instructions. Transfected cells were incubated for 24 to 72 hr. following transfection after which the supernatant was harvested and filtered through a 0.45 μM Nalgene filter. The vector particle-containing supernatant was either used immediately for infection, or further concentrated and/or purified as described in Example 13. Vector particles not used immediately were stored in conditioned media or freezing buffer containing lactose at −70° C. until further use.

Titer of FIV vector particles was determined by the addition of serial dilutions of VSV-G pseudotyped FIV vector particles prepared as above (before or after concentration) to HT1080, 3T3, 293T, CrFK or other cells of interest in culture media containing 8 ug/ml polybrene. The cultures were incubated for 48 to 72 hr following initial infection and then assayed for expression of the transduced gene. β-galactosidase gene (lacZ) expression was assayed after removing the medium and fixing the cells in cold 2% formaldehyde/0.2% glutaraldehyde in PBS for 5 min. The cells were washed twice with PBS and stained with fresh X-gal staining solution consisting of 1 mg/ml X-gal, 5 mM potassium ferricyanide, 5 mM potassium ferrocyanide and 2 mM MgCl₂ in PBS for 50 min at 37° C. The cells were again washed with PBS and the titer determined from the number of blue foci per well. Enhanced green fluorescent protein gene expression was assayed by flow cytometry after fixing the cells in 4% formaldehyde and washing with PBS.

### Example 9

### DETERMINATION OF FIV VECTOR CONSTRUCT REQUIREMENTS

To delineate the minimal cis-acting vector construct requirements for efficient transduction of dividing cells, four FIV vector constructs expressing β-galactosidase from an internal CMV promoter were tested in conjunction with an FIV packaging construct (pCFIX, Example 5A) and the VSV-G env plasmid, pCMV-G. The FIV vector, pTFLC, (Example 3F), contains the authentic FIV 5′ LTR, adjacent non-coding region and approximately 0.5 kb corresponding to the Gag coding region. pTC/FLCD (Example 3N) is similar to pTFLC, except that the entire U3 region of the FIV 5′ LTR is replaced with the CMV promoter. pTC/FSC, (Example 3M) also contains the CMV promoter in place of the FIV U3 region, however contains only approximately 0.3 kb corresponding to the Gag coding region. pTC/FLCβCTE (Example 30) is identical to pTC/FLCβ with an additional constitutive transport element (CTE) element from Mason-Pfizer monkey virus (MMPV; Bray et al., PNAS 91:1256, 1994).

Supernatants from 293T cells transfected with the FIV packaging construct, pCMV-G and one of the four FIV vector constructs were assayed for transduction efficiency in HT1080 cells (Table 2). Vector particles produced from the vector containing the authentic FIV 5′ LTR (pTLCO) yielded titers of 5.3×10⁴ LFUI/ml in contrast to vectors produced from CMV promoter/FIV hybrid LTR vectors (pTC/FLCO, pTC/FSCβ and pTC/FLCβCTE) which yielded titers of approximately 3×10⁶ LFU/ml virus stock. Analysis of FIV p24 capsid levels using the IDEXX FIV antigen test kit (Portland, Maine) indicated no significant differences between p24 levels in the supernatants of cells transfected with the hybrid or non-hybrid LTR vectors (data not shown), however, since FIV p24 is produced from the packaging construct, any differences would likely be minimal with the same packaging construct being used for all of the transfections. The 60-fold drop in titer observed in repeated experiments using vectors containing the complete FIV 5′ LTR suggests a requirement in human cells for additional cis-acting signals not normally present in the FIV LTR for enhanced transcriptional activity.

TABLE 2
Effect of FIV vector construct on vector titer

| Packaging | Vector | Mean Titer^{a} |
|---|---|---|
| Construct | Construct | (LFU/ml virus stock)^{b} |
| pCFIVX | pTC/FLCβ | 3.2 × 10⁶ |
| pCFIVX | pTC/FLCβ | 3.1 × 10⁶ |
| | CTE | |
| pCFIVX | pTC/FSCβ | 3.1 × 10⁶ |
| pCFIVX | pTFLCβ | 5.3 × 10⁴ |

^{a}Results are from a representative experiment with each value representing the average titer of three replicate vector preparations. At least three experiments done in triplicate were performed.
^{b}Vector titer is expressed as lacZ forming units per ml (LFU/ml) virus stock. Pseudotyped FIV vector stocks were generated by transient transfection in 293T cells and the resulting supernatant used to transduce HT1080 cells. Titer of vector stocks was measured by counting the number of blue foci following X-gal staining.

To verify that the β-galactosidase activity observed in the transduced cells is due to expression following reverse transcription and not the result of pseudotransduction of β-galactosidase activity present in the vector preparations, HT1080 cells were transduced in the presence or absence of 3′-azido-3′deoxythymidine (AZT). The titer resulting from transduction of cells in the presence of AZT was 0.5% or less of that observed in cells infected in the absence of AZT, both in the case of vectors containing hybrid as well as nonhybrid promoters. These data suggest that nearly all of the β-galactosidase activity is the result of true transduction by the FIV vectors.

### Example 10

### DETERMINATION OF FIV PACKAGING CONSTRUCT REQUIREMENT

FIV packaging constructs were developed to minimize the amount of sequence homology between packaging and vector constructs as well as to eliminate the production of proteins dispensable for efficient vector production and infectivity. Five packaging constructs differing either in 5′ noncoding sequence or ability to express accessory genes were analyzed in conjunction with an FIV vector construct expressing β-galactosidase (pTC/FLCβ, Example 3N) and the env plasmid, pCMV-G. The FIV packaging construct pCFIVX (Example 3A) contains approx. 100 bp of noncoding sequence upstream of the major splice donor site while the packaging construct pCFIV (Example 5E) contains only 6 bp of noncoding sequence upstream of the splice donor site and lacks 17 bp normally located between the splice donor site and the start codon for gag. In addition, the pCFIV packaging construct contains an intact orf2 corresponding to that of FIV14 (Olmsted et al., PNAS 86:8088, 1989). In contrast, the pCFIVΔORF2 packaging construct (Example SC), derived from FIV-34TF10, contains a premature stop codon within orf2 and, thus, does not give rise to a functional ORF2 product (Talbott et al., PNAS 86:5743,1989; Phillips et al., J. Virol. 64:4605, 1990). pCFIVΔVIF (Example 5F) contains a premature stop codon in the Vif coding region followed by a frameshift mutation corresponding to amino acid 22. pCFIVΔORF2ΔVIF contains mutations in the Orf2 and Vif coding regions (Example 5G).

Each of the packaging plasmids were separately transfected into 293T cells together with FIV vector (pTC/FLCβ) and env plasmid pCMV-G and the supernatants assayed for transduction efficiency (Table 3). All five packaging constructs analyzed yielded similar titers ranging between 2.9×10⁶ and 3.2×10⁶ LFU/rl. Analysis of p24 levels in the supernatants also indicated similar levels of p24 production from the packaging constructs tested (data not shown). Comparison of titer of FIV vector particles prepared from the packaging constructs containing differing lengths of FIV 5′ noncoding sequence (pCFIVX and pCFIV) indicates that the deleted sequences are not required for the efficient translation of FIV proteins required in trans for particle production. In addition, the introduction of mutations in the coding regions for the FIV accessory genes orf2 and vif either singly or in combination, had no substantial effect on transduction efficiency in HT1080 cells (Table 3).

TABLE 3
Effect of packaging construct on vector titer

| Packaging | Vector | Mean Titer^{a} |
|---|---|---|
| Construct | Construct | (LFU/ml virus stock)^{b} |
| pCFIV | pTC/FL | 3.1 × 10⁶ |
| | Cβ | |
| pCFIVΔORF2 | pTC/FL | 2.9 × 10⁶ |
| | Cβ | |
| pCFIVΔVIF | pTC/FL | 3.3 × 10⁶ |
| | Cβ | |
| pCFIVΔORF2 | pTC/FL | 3.0 × 10⁶ |
| ΔVIF | Cβ | |

^{a}Results are from a representative experiment with each value representing the average titer of three replicate vector preparations. At least three experiments done in triplicate were performed.
^{b}Vector titer is expressed as lacZ forming units per ml (LFU/ml) virus stock. Pseudotyped FIV vector stocks were generated by transient transfection in 293T cells and the resulting supernatant used to transduce HT1080 cells. Titer of vector stocks was measured by counting the number of blue foci following X-gal staining.

### Example 11

REQUIREMENT FOR REY/RRE IN FIV VECTOR AND PACKAGING CONSTRUCTS

To ascertain whether the FIV rev regulatory gene as well as the Rev-responsive element (RRE) to which it binds is necessary for efficient particle production and subsequent transduction, FIV packaging constructs were generated which either do not express rev or lack the Rev coding regions (Example 5H). In the FIV packaging construct, pCFIVΔrev, the splice acceptor site and the basic amino acid domain of the second exon of rev were deleted (Phillips et al., J. Virol. 66:5464, 1992). A similar deletion of the splice acceptor site to create an FIVΔrev construct was previously demonstrated to yield insignificant levels of infectious virus. In the FIV packaging constructs pCFIVFRRE and pCFIVBRRE, the coding regions for Rev (as well as Vif and Orf2) were deleted and the RREs from FIV or HIV, respectively, introduced (Example 5H). The Δrev FIV packaging constructs were tested together with various FIV vector constructs designed to investigate whether the type, location and inclusion of an additional export element could influence the relative transduction efficiency of FIV vector particles. In addition to the FIV packaging constructs containing the FIV or WHIV RRE, FIV vector constructs were generated that contain the FIV or mV RREs upstream of the internal CMV promoter driving expression of the β-galactosidase gene (pTC/FSCβFR and pTC/FSCβHR, respectively; Example 3S). FIV vector constructs were also generated that lack the FIV RRE (pTC/FLβCΔRRE, Example 3S) or that contain the MPMV CTE export element (pTC/FLCβCTEΔRRE, Example 3S). The location of the FIV and HIV RREs in these constructs differs from that of the previous constructs (e.g., pTC/FLCβ, Example 3N) in which the FIV RRE is located downstream of the β-galactosidase gene. To complement the FIV rev-deleted packaging constructs, a construct expressing FIV rev from a CMV promoter (pCFIVrev; de Parseval et al., J. Virol. 73:608-617, 1999) was included in the cotransfections. A construct expressing the HIV rev from a CMV promoter (pCHIVrev-called pCMVrev- , NIH Research and Reference Reagent Program, Cat# 1443) was cotransfected into producer cells to complement the HIV rev-deleted packaging construct.

The titer of vector particles generated in the absence of FIV rev in the packaging construct dropped dramatically compared to that of particles prepared in the presence of FIV rev. Titers of vector particles generated in the presence of FIV rev using a vector construct lacking the FIV RRE, were reduced compared to those using an FIV vector containing the FIV RRE. Titers of FIV vector constructs containing the FIV RRE upstream of the internal CMV promoter were slightly higher than those generated using vector constructs containing the FIV RRE downstream of the β-galactosidase gene. The latter result indicates that the location of the export element in the vector construct can have a moderate impact on transduction efficiency.

Titers resulting from cotransfection of a rev expression plasmid (pCFIVrev) together with an FIV packaging construct which does not express rev were lower than titers resulting from cotransfection using the pCFIV rev expressing packaging construct. Titers resulting from a similar cotransfection using an FIV packaging construct lacking both exons of rev, however, were considerably reduced compared to those resulting from cotransfection using the pCFIV rev-expressing packaging construct. The substantial reduction in titer was not significantly compensated for by the use of an FIV vector construct containing the FIV RRE upstream of the internal cassette. Substitution of the FIV RRE with that of the HIV RRE in the FIV rev deleted packaging construct restored titers to the level observed using an FIV packaging construct lacking only the rev splice acceptor and basic binding domain. This data indicates that the HIV rev/RRE combination might give rise to highter titers than the FIV rev/RRE combination in human cells.

TABLE 4
Requirement of Rev/RRE for Efficient Transduction of HT1080 Cells

| | | | Mean |
|---|---|---|---|
| Packa | | | Transduction |
| ging | Vector | Rev | Efficiency^{a} |
| Construct | Construct | Construct | (LFU/ml virus stock)^{b} |
| pCFIV | pTC/FSCβ | none | 2.6 × 10⁶ |
| pCFIV | pTC/FSCβΔRR | none | 9.3 × 10⁵ |
| | E | | |
| pCFIV | pTC/FSCβFRR | none | 3.4 × 10⁶ |
| | E | | |
| pCFIV | pTC/FSCβ | pCFIV | 2.2 × 10¹ |
| Δrev | | rev | |
| pCFIV | pTC/FSCβ | pCFIV | 1.1 × 10⁶ |
| Δrev | | rev | |
| pCFIV | pTC/FSCβ | pCFIV | 1.0 × 10⁵ |
| FRRE | | rev | |
| pCFIV | pTC/FLCβFRR | pCFIV | 1.6 × 10⁵ |
| FRRE | E | rev | |
| pCFIV | pTC/FLCβHRR | pCHI | 1.0 × 10⁶ |
| HRRE | E | Vrev | |

^{a}Results are from a representative experiment with each value representing the average titer of three replicate vector preparations. At least three experiments done in triplicate were performed.
^{b}Vector titer is expressed as lacZ forming units per ml (LFU/ml) virus stock. Pseudotyped FIV vector stocks were generated by transient transfection in 293T cells and the resulting supernatant used to transduce HT1080 cells. Titer of vector stocks was measured by counting the number of blue foci following X-gal staining.

### Example 12

### INFECTION OF NON-PROLIFERATING CELLS BY FIV VECTOR PARTICLES

To study whether expression of the FIV accessory genes, vif and orf2 are required for the transduction of nondividing cells, a variety of cells were transduced with FIV vectors and transduction efficiencies analyzed.

A. Transduction of Primary Human Skin Fibroblasts

Primary human skin fibroblasts (HSF) were arrested at the G₀/G₁ phase of the cell cycle by density-dependent inhibition of growth. The arrested cells were then transduced with FIV vectors prepared using each of the packaging constructs expressing both, either or neither FIV vif and orf2 (pCFIV, pCFIVΔORF2, pCFIVΔVIF and pCFIVΔORF2ΔVIF, respectively) together with an FIV β-galactosidase expressing vector construct (pTC/FLCβ) and pCMV-G envelope plasmid. Transduction efficiencies of growth arrested HSF cells were approximately 2-fold lower than those obtained from proliferating HSF cells (e.g., 3.2×10⁴ for dividing cells compared to 1.6×10⁴ for nondividing cells; Table 5). An MLV vector, also containing an internal CMV promoter driving expression of the β-galactosidase gene, efficiently transduced dividing HSF cells. However, in contrast to transduction of nondividing HSF cells by FIV vectors, the transduction efficiency of the MLV vector in nondividing HSF cells was dramatically reduced (from 3.8×10⁴ in dividing cells to 7.3×10¹ in nondividing cells; Table 5). These data indicate that, unlike MLV vectors, FIV vectors are capable of efficiently transducing nonproliferating HSF cells and, in addition, that FIV vif and orf2 accessory gene expression is not required for efficient transduction.

TABLE 5
Effect of Accessory Gene Expression on Transduction of
Human Primary Skin Fibroblasts

| | | Mean Transduction | |
|---|---|---|---|
| | | Efficiency^{a} (LFU/ml | |
| Packaging | Vector | of virus stock)^{b} | |
| Construct | Construct | Dividing | Nondividing |
| pCFIV | pTC/FLCβ | 3.15 × 10⁴ | 1.60 × 10⁴ |
| pCFIVΔORF2 | pTC/FLCβ | 3.28 × 10⁴ | 1.69 × 10⁴ |
| pCFIVΔVIF | pTC/FLCβ | 3.28 × 10⁴ | 1.70 × 10⁴ |
| pCFIVΔORF2 | PTC/FLCβ | 3.29 × 10⁴ | 1.73 × 10⁴ |
| ΔVIF | | | |
| pMLVgagpol | PTC/FLCβ | 3.81 × 10⁴ | 7.30 × 10¹ |

^{a}Results are from a representative experiment with each value representing the average titer of three replicate vector preparations.
^{b}Vector titer is expressed as lacZ forming units per ml (LFU/ml) virus stock. Pseudotyped FIV vector stocks were generated by transient transfection in 293T cells and the resulting supernatant used to transduce HT1080 cells. Titer of vector stocks was measured by counting the number of blue foci following X-gal staining.

B. Transduction of Growth-Arrested HT1080 Cells

To study the effect of vif and orf2 accessory gene expression on transduction of another nondividing cell type at various multiplicities of infection, HT1080 cells were exposed to y-irradiation to arrest the cells at the G₂ phase of the cell cycle. Proliferating or growth arrested HT1080 cells were infected with vector prepared from packaging constructs expressing both, one, or neither vif and orf2 accessory gene (pCFIV, pCFIVΔORF2, pCFIVΔVIF or pCFIVΔORF2ΔVIF, respectively) together with pCMV-G and a vector construct expressing the enhanced green fluorescent protein gene under the control of the MLV promoter (pTC/FLMEGFP). Cells were infected at MOIs of 2.0, 0.2 or 0.02 and the percentage of cells expressing the enhanced green fluorescent protein gene was determined by flow cytometry. At all MOIs, little difference in transduction efficiency was observed in dividing or nondividing cells infected with FIV vector prepared with or without accessory gene expression (Table 6). An MEV vector expressing the enhanced green fluorescent protein gene also efficiently transduced dividing HT1080 cells at all MOIs tested, however, even at a very high MOI (ie., MOI=10), the MLV vector failed to efficiently transduce growth arrested HT1080 cells. These data again demonstrate that FIV vectors can efficiently transduce quiescent cells and the FIV vif and orfl accessory gene expression is not required to infect these cells even at a low MOI (i.e., MOI=0.02). In addition, because the transduction efficiency of the VSV-G pseudotyped MLV vector is comparable to background levels in nondividing cells (ie., the level observed in the absence of vector), these data indicate that the observed transduction is not due to pseudotransduction of green fluorescent protein activity which could be present in the vector preparations.

TABLE 6
Effect of Accessory Gene Expression On Transduction Efficiency Of
Dividing and Irradiated HT1080 Cells

| Packaging | | Mean % Transduced Cells | |
|---|---|---|---|
| Construct | MOI | Dividing | Irradiated |
| pCFIV | 2.0 | 81.14% | 79.08% |
| | 0.2 | 38.54% | 45.51% |
| | 0.02 | 4.76% | 5.97% |
| pCFIVΔ | 2.0 | 74.82% | 78.85% |
| ORF2 | 0.2 | 23.96% | 40.24% |
| | 0.02 | 3.31% | 8.27% |
| pCFIVΔ | 2.0 | 76.20% | 76.28% |
| VIF | 0.2 | 22.24% | 39.03% |
| | 0.02 | 3.82% | 7.55% |
| pCFIVΔ | 2.0 | 74.51% | 72.48% |
| ORF2ΔVIF | 0.2 | 23.20% | 41.40% |
| | 0.02 | 3.45% | 6.31% |
| pMLVga | 10 | 97.87% | 0.05% |
| gpol | 2.0 | 79.35% | 0.54% |
| | 0.2 | 27.34% | 0.41% |
| | 0.02 | 3.95% | 0.44% |
| None | N/A | 0.19% | 0.24% |

C. Transduction of Quiescent CD4⁺ T lymphocytes

To determine whether pseudotyped FIV vectors are capable of transducing quiescent peripheral blood lymphocytes (PBL) or quiescent CD4+ T lymphocytes following activation, EGFP expressing FIV vectors were used to infect either population. PBLs were harvested from the blood of healthy donors and purified by centrifugation over FicolYHypaque as above. PBLs were washed extensively with PBS to remove platelets and depleted of monocytes by passage over plastic. CD4+T lymphocytes were purified from freshly harvested PBLs using Detachabead following manufacturers instructions (Dynal Inc.). Lymphocytes were maintained in RPMI containing 10% human serum and the activation state of the cells determined by ³T-thymidine incorporation prior to infection. VSV-G pseudotyped FIV vectors capable of expressing EGFP were incubated together with the cells for 8 to 24 hr after which the cells were washed extensively. The lymphocytes were then activated using 3 mg/ml PHA and 10U/ml IL2 and maintained for 2 to 5 days before the transduction efficiency was measured by flow cytometry. The low but measurable percentage of transduced CD4+T lymphocytes (Table 7) indicates that FIV vector particles prepared in the presence or absence of accessory gene expression were capable of entering quiescent T cells and of expressing EGFP upon induction of the cells. In contrast, MLV vector particles were unable to efficiently transduce the cells, even after activation (Table 7), presumably due to the inability of the MLV vector to form a stable infectious intermediate, which could enter the nucleus of the infected cells upon activation.

TABLE 7
Transduction Of Quiescent T Lymphocytes With FIV-Based Vectors
With and Without Accessory Proteins

| Packaging | | % Transduced Cells |
|---|---|---|
| Construct | 293T | CD4+ T lymphocytes |
| pCFIV | 45.42% | 4.11% |
| pCFIVΔORF2 | 43.24% | 3.04% |
| pCFIVΔVIF | 42.59% | 3.00% |
| pCFIVΔORF2 | 46.70% | 3.43% |
| ΔVIF | | |
| pMLVgagpol | 44.63% | 0.05% |
| None | 0.13% | 0.07% |

D. Transduction of Terminally Differentiated Human Macrophages

To determine whether pseudotyped FIV vectors are capable of transducing terminally differentiated primary cells, luciferase-expressing FIV vectors were used to infect human monocyte-derived macrophages. Monocytes were harvested from the blood of healthy donors and purified by centrifugation over Ficoll/Hypaque (Kornbluth et al., J. Exp. Med. 169:1137, 1989). Monocytes were further purified by adherence to plastic and maintained in RPMI containing 10% human serum for 7 to 14 days. VSV-G pseudotyped FIV vectors capable of expressing luciferase were then used to infect the terminally differentiated macrophages and the transduction efficiency measured by luciferase assay according to the manufacturers instructions (Promega Corp., Madison, Wis.). As above, unlike MLV-based particles, FIV vector particles were able to transduce terminally differentiated macrophages whether the particles were prepared in the presence or absence of FIV vif and orf2 gene expression (Table 8).

TABLE 8
Transduction of Monocyte-Derived Macrophages With FIV-Based
Vectors With and Without Accessory Proteins

| | | Luciferase Activity (RLU) | |
|---|---|---|---|
| Packaging | | monocyte-derived macrophage | |
| Construct | 293T | Donor A | Donor B |
| pCFIV | 255,042 | 2,226 | 846 |
| pCFIVΔORF | 267,895 | 1,193 | 660 |
| 2 | | | |
| pCFIVΔVIF | 298,275 | 693 | 766 |
| pCFIVΔORF | 383,093 | 710 | 631 |
| 2ΔVTF | | | |
| pMLVgagpol | 146,905 | 5.95 | 6.05 |
| None | −1.89 | −1.90 | −1.42 |

### Example 13

### TRANSDUCTIUION OF HEMATOPOIETIC PROGENITOR CELLS

A. Transduction of Mouse Hematopoietic Progenitor Cells (HPC)

The FIV vectors of the present invetion encoding an eGFP reporter gene were constructed and produced as described herein and according to Johnston, J. C. et al. 1999. J. Viol. 73(6) 4991-5000. The FIV-eGFP vector was pseudotyped with the VSV-G envelope and the final titers were in the range of ˜1×10⁸-10⁹ cfu/ml.

C57B1/6 mice were purchased from the Jackson Laboratory (Bar Harbor, Me.) at 5-10 weeks of age. Bone marrow was harvested from the femurs and tibiae of donor mice. HPCs were enriched from the erythrocyte-depleted bone marrow using the StemSep™ magnetic negative selection system (Stem Cell Technologies, Vancouver, Canada) according to the manufacturer's instructions. The percentage of Sca-1⁺ cells after enrichment was ˜20% as determined by FACS analysis.

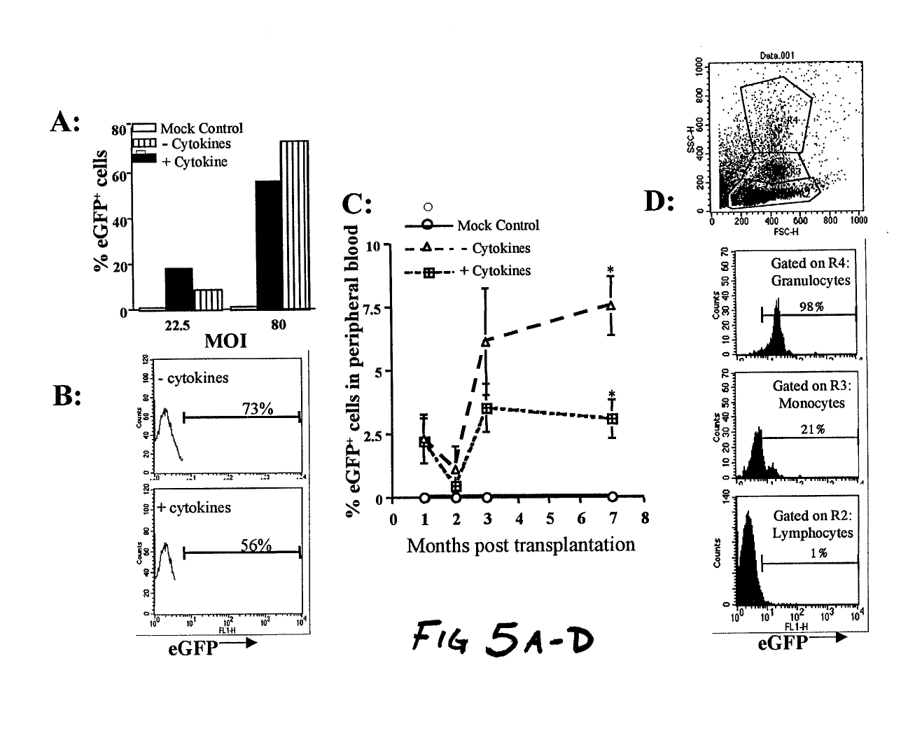

The present invetiors previously demonstrated that the FIV-based lentiviral vectors efficiently transduced fully differentiated airway epithelia, (Wang, G. et al. 1999. 104:R55-R62) hamster muscle (Johnston, J. C. et al. 1999. J. Viol. 73(6) 4991-5000) and hepatocytes. This example demonstrates that vectors of the present invetion can also transduce HPCs. Enriched murine Sca-l⁺ Lin7 HPCs were transduced with FIV-eGFP in the presence or absence of exogenous cytokines. As shown in FIG. 5A, the FIV-based vector transduced murine HPCs in a dose-dependent manner. At a multiplicity of infection (MOI) of 22.5, the percentage of eGFP-expressing cells was −10%, while at an MOI of 80, the percentage of eGFP⁺ cells increased to −70%. FIV vector transduction efficiencies in the absence of cytokine stimulation were comparable to those in cells that were pre-stimulated with cytokines (FIGS. 5A and 5B). The ability of FIV-based vectors to transduce hematopoietic progenitor cells in the absence of cytokine pre-stimulation may minimize cell cycling and maintain in vivo repopulating capacity.

To further examine the long-term reconstitution capacity of the transduced HPCs, the cells were injected into irradiated syngenic mice and the eGFP⁺ cells in peripheral blood were assayed. As shown in FIG. 5C, eGFP expression in the recipient mice persisted for at least seven months, the last time point examined, demonstrating the ability of the FIV vector to target long-term repopulating HPCs. At 1 month post transplantation, the percentage of eGFP⁺ cells was comparable between groups of mice receiving cells transduced with or without cytokine pre-stimulation. However, by seven months post transplantation, the percentage of eGFP⁺ cells was significantly higher in the mice receiving cells transduced in the absence of cytokines (p<0.05).

To characterize the cell types expressing eGFP, peripheral nucleated cells were sub-divided into granulocyte, monocyte and lymphocyte populations based on parameters of forward side scatter (FSC) and side side scatter (SSC). Results from a representative animal are shown in FIG. 5D. EGFP expression was detected in the myeloid lineage (granulocytes and monocytes), but not in the lymphoid cells.

B. Transduction of Human Hematopoietic Progenitor Cells (HPC)

The FIV vectors of the present invetion encoding an eGFP reporter gene was constructed and produced as described herein and according to Johnston, J. C. et al. 1999. J. Viol. 73(6) 4991-5000. The FIV-eGFP vector was pseudotyped with the VSV-G envelope and the final titers were in the range of ˜×10⁸-10⁹ cfu/ml

Human CD34⁺ cells were enriched from umbilical cord blood using a StemSep™ rosette or a magnetic negative separation device (Stem Cell Technologies) according to the manufacturer's instructions. The percentage of CD34⁺ cells after enrichment was ˜50% as determined by FACS analysis.

The enriched HPCs were either immediately transduced in the absence of any exogenous cytokines or pre-stimulated with SCF (100 ng/ml), IL-3 (50 ng/ml) and IL-6 (50 ng/ml) for 2 days prior to transduction. Centrifugation was used to enhance the transduction efficiency (Bahnson, A. B. 1995. J. Virol. Meth. 54:131-143). Briefly, 1×10⁵ cells in a 2 ml volume were added per 15-ml culture tube containing various concentrations of the FIV-eGFP vector, and centrifuged for 2 h at 2400 g at 20° C. After centrifugation, the cells were incubated for an additional 3-4 hours without replacement of medium and without transfer from the culture tubes. The cells were washed and subjected to the assays described below.

An aliquot of transduced or mock-transduced cells (approximately 1×10⁵ cells) was incubated in culture medium containing recombinant cytokines (SCF: 100 ng/ml; IL-3: 50 ng/ml; IL-6: 50 ng/ml). Half of the medium was replaced every two days, and the transduction efficiency was determined by assaying eGFP expression by FACS or fluorescence microscopy on days 6-8. In another series of experiments, irradiated (600-1100 rad) syngenic mice received transduced cells (2-5×10⁵cells) by tail vein injection and the percentage of eGFP⁺ cells in peripheral blood was measured by FACS analysis.

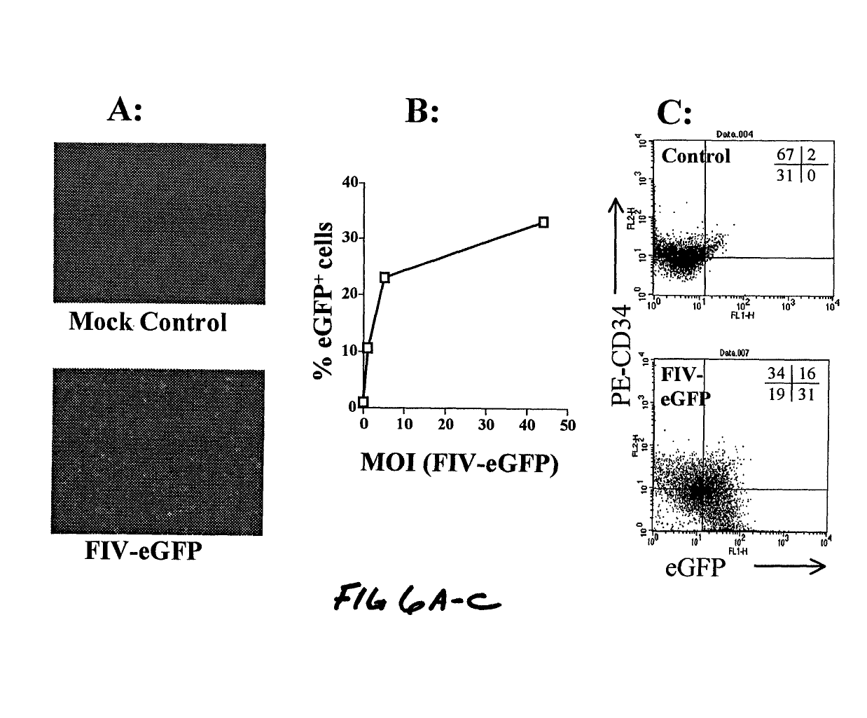

To examine the efficiency of FIV-mediated gene transfer to human HPCs, CD34⁺ cells were isolated from umbilical cord blood and transduced with FIV-eGFP in vitro. The cells were then cultured for seven days and eGFP expression was measured by FACS and fluorescence microscopy. As shown in FIG. 6A, FIV readily transduced human HPCs. Consistent with our results with murine HPCs, the FIV-based vector transduced human CD34⁺ cells in a dose-dependent manner (FIG. 6B). Moreover, about 34% of eGFP⁺ cells maintained the CD34⁺ phenotype (FIG. 6C). These results suggest human CD34⁺ cells are efficiently transduced by FIV lentiviral vectors.

### Example 14

### FIV VECTOR PARTICLE PURIFICATION AND CONCENTATION

Retroviral vectors can be purified and concentrated by a number of means, including PEG precipitation, centrifugation, ultrafiltration, ion exchange chromatography, size exclusion chromatography, affinity chromatography and sucrose gradient (Aboud et al., Arch. Virology 71:185-195, 1982; U.S. Pat. No. 5,661,022, Bowles et al., Human Gene Therapy 7:1735-1742, 1996; U.S. Pat. No. 5,447,859). In addition, viral particles can potentially be concentrated during a lyophylization process (U.S. Pat. No. 5,792,643). In this example, we are describing unique procedures for the purification and concentration of FIV vectors. Pseudotyped FIV vectors were transiently produced in 293T cells via CaPO4-transfection as described in Example 8.

A. Concentration of pVETT_{L}ChGH and pVETT_{L}MEpo FIV vector particles

This example describes the concentration of FIV vector particles by PEG precipitation followed by purification on an ion exchange column. 293T cells were transiently transfected with the pVET_{L}ChGH (Example 3AB) or pVET_{L}Mepo (Example 3V) hybrid FIV LTR vectors, the FIV packaging expression constructs pCFIV (Example 5E) or pCFIVΔORF2ΔVIF (Example 5G) and a VSV-G envelope-expressing plasmid via CaPO₄ transfection (Profectin kit, Promega Corp., Madison, Wis.). The supernatant from both vectors was pooled, filtered (0.45 um Nalgene filter) and precipitated for at least 6 h at 4° C. in the presence of 10% PEG final concentration (polyethylene glycol in PBS). The PEG-precipitate containing the viral particle was pelleted by centrifugation for 15 minutes at 3,000 rpm in a Sorvall tabletop centrifuge. The pellet was resuspended in PBS and the equivalent of 50 ml crude supernatant applied to a DEAE column (Toyopearl DEAE-650C, Tosohaas, Montgomeryville, PA) with 4 ml bed volume. The column was then washed with PBS and the virus eluted with PBS/500 mM NaCl. The FIV vector particles were spun down in an Eppendorf centrifuge for 1 hour at 4° C. and 14,000 rpm. The pellet was resuspended in PBS and stored at −80° C. in small aliquots.

The titer of the FIV vector preparation was determined by transducing HT-1080 target cells with a range of dilutions of the viral preps. The general transduction procedure is described in Example 9. Two days post-transduction, 100 ul supernatant of the transduced cells was used to determine the amount of secreted hGH in duplicates using the HGH-TGES detection kit (Nichols Institute Diagnostics, San Juan Capistrano, Calif.). The titer of the pVET_{L}ChGH vector preparation was compared to an MLV-based hGH-expressing vector standard with a known titer. The titer of the pVET_{L}Mepo vector was determined as described in Example 13B.

TABLE 9
Concentration of pVET_{L}ChGH vector particles

| pVET_{L}ChGH | | | | % |
|---|---|---|---|---|
| vector | Amount | Titer | Concentration | Overall |
| material | nt (ml) | (CFU/ml) | (x-fold) | recovery |
| Crude | 790 | 2.2E5 | | |
| supernatant | | | | |
| Final | 3.5 | 2.4E7 | 110 | 48% |
| preparation | | | | |

TABLE 10
Concentration of pVET_{L}MEpo vector particles

| pVET_{L}MEpo | | | Concen- | % | % |
|---|---|---|---|---|---|
| vector | Amount | Titer | tration | step | Overall |
| material | nt (ml) | (CFU/ml) | (x-fold) | recovery | recovery |
| Crude | 4000 | 3.8E4 | | | |
| Clarified | 4000 | 3.1E4 | 1 | 81.6 | |
| PEG | 325 | 4.2E5 | 12.3 | 110.1 | |
| concentrate | | | | | |
| DEAE | 50 | 2.7E6 | 80 | 76.9 | |
| eluate | | | | | |
| Centrifuged | 4 | 1.8E7 | 1000 | 53.3 | 36.8 |

B. Concentration of pVET_{L}MEpo FIV vector particles

This example describes the concentration of FIV vector particles by a centrifugation alone. 293T cells were transiently transfected with the pVET_{L}MEpo 10 hybrid FIV LTR vector (Example 3V), the FIV packaging expression construct pCMVFIV (Example 5E) and a VSV-G envelope-expressing plasmid via CaPO₄ transfection (Profectin kit, Promega Corp., Madison, Wis.). The crude vector preparation was clarified by a 0.45 μm filtration step. The clarified sample was further concentrated by centrifugation at 6,000 rpm using a GS-3 rotor (Sorvall). After the centrifugation was completed, the supernatant that contains the majority of the cellular and FBS derived proteins was removed, and the pellets containing FIV viral vector were resuspended in PBS/lactose buffer. The titer of the samples was estimated by measuring the expression level in the transduced HT-1080 cells using the EPO detection kit (Quantikine IVD kit, R&D Systems, Inc., Minneapolis, Minn.) and shown in the following table.

TABLE 11
Concentration of pVET_{L}MEpo vector particles

| pVET_{L}MEpo | | | Concen- | % | % |
|---|---|---|---|---|---|
| vector | Amount | Titer | tration | step | Overall |
| material | nt (ml) | (CFU/ml) | (x-fold) | recovery | recovery |
| Crude | 3200 | 1.2E5 | | | |
| Clarified | 3200 | 1.1E5 | 1 | 92 | |
| Centrifuged | 4 | 4.1E5 | 800 | 47 | 43 |

C. Concentration of pVETT,CEpo vector

In this example, FIV vector particles were concentrated by PEG precipitation followed by a centrifugation step. 293T cells were transiently transfected with the pVET_{L}CEpo hybrid FIV LTR vector (Example 3X), the FIV packaging expression construct pCFIV (Example 5E) and a VSV-G envelope-expressing plasmid via CaPO₄ transfection (Profectin kit, Promega Corp., Madison, Wis.). The crude vector preparation was clarified by a 0.45 μm filtration step. The clarified sample was treated with 10% PEG overnight before the precipitates that contain the FIV vector particles were collected by centrifugation (6,000 rpm for 10 minutes in a GS-3 rotor, Sorvall). The PEG pellet was resuspended in PBS/lactose buffer and then subjected to another round of centrifugation to pellet the FIV vector particles. This final centrifugation was done at 6,000 rpm for 18-24 hours using the GS-3 rotor (Sorvall). The pellet was resuspended in PBS/lactose buffer. The titer of the samples were estimated as described in the Example 13B and shown in the following table.

TABLE 12
Concentration of pVET_{L}CEpo vector particles

| pVET_{L}CEpo | | | Concen- | % | % |
|---|---|---|---|---|---|
| vector | Amount | Titer | tration | step | Overall |
| material | nt (ml) | (CFU/ml) | (x-fold) | recovery | recovery |
| Crude | 5000 | 3.1E5 | | | |
| Clarified | 5000 | 6.8E5 | 1 | 221 | |
| PEG | 200 | 1.1E7 | 25 | 62.9 | |
| concentrate | | | | | |
| Centrifuged | 3 | 2.0E8 | 1667 | 27.9 | 38.8 |

Above procedure allowed crude material vector preparation to be concentrated up to 2000-fold and produced very high titer samples which is critical for efficient in vitro and in vivo transductions.

### Example 15

### CONSTRUCTION OF FIV VECTORS WITH MINIMAL PACKAGING SIGNAL

To obtain an FIV vector construct with minimal 5′ sequences as well as map the location of the functional FIV packaging signal, a series of FIV hybrid LTR vector constructs were generated which contain progressively smaller portions of the Gag coding region and adjacent 5′ noncoding region. To accomplish this, fragments corresponding to the Gag coding region and upstream noncoding region were amplified by PCR, digested with Nar I and Not I and exchanged for the Nar I/Not I fragment of pTC/FSCβ (Example 3 M).

A. Construction of pVET_{S}Cβ(740)

The hybrid FIV LTR vector pVET_{S}Cβ(740) is constructed as follows. Primers for the amplification of a fragment containing the entire 5′ noncoding region and 0.1 kb of Gag coding region are Nar(+) (SEQ ID No. 50; GCC GAG AAC TTC GCA GTT GG) and Gag740(−) (SEQ ID No. 51; AGC TGA CAA ATA GCG GCC GCC CAT CTG AAA TTC CCT T). As a template, the FIV hybrid LTR vector pTC/FLCβ (Example 3N) is used. The resulting fragment is digested with Nar I and Not I and inserted into similarly digested pTC/FSCβ (Example 3M) to yield pVET_{S}Cβ(740).

B. Construction of pVET_{S}Cβ(627)

The hybrid FIV LTR vector pVET_{S}Cβ(627) is constructed as follows. Primers for the amplification of a fragment containing the entire 5′ noncoding region up to but not including the start codon for Gag are Nar(+) (SEQ ID No. 50; Example 14A) and NC627(−) (SEQ ID No. 52; AGC TGA CAA ATA GCG GCC GCG TTG CTG TAG AAT CTC TCC). As a template, the FIV hybrid LTR vector pTC/FLβ (Example 3N) is used. The resulting fragment is digested with Nar I and Not I and inserted into similarly digested pTC/FSCβ (Example 3M) to yield pVET_{S}Cβ(627).

C. Construction of pVET_{S}Cβ(540)

The hybrid FIV LTR vector pVET_{S}Cβ(540) is constructed as follows. Primers for the amplification of a fragment containing the 0.18 kb of 5′ noncoding region are Nar(+) (SEQ ID No. 50; Example 14A) and NC540 (SEQ ID No. 53; AGC TGA CAA ATA GCG GCC GCT TAT CTG GGC CTT TAA ACA ATG). As a template, the FIV hybrid LTR vector pTC/FLCβ (Example 3N) is used. The resulting fragment is digested with Nar I and Not I and inserted into similarly digested pTC/FSCβ to yield pVET_{S}Cβ(540).

D. Construction of pVET_{S}Cβ(369)

To generate a construct containing the complete FIV hybrid LTR but lacking any 5′ untranslated region, pTC/FSCP (Example 3M) is digested with Nar I and Not I, the vector treated with mung bean nuclease and religated to yield pVET_{S}Cβ(360). Also included in the series are FIV constructs pTC/FLC3 (Example 3N) and pTC/FSCP (Example 3M) which contain 0.5 kb and 0.3 kb of Gag coding sequence, respectively.

### Example 16

CONSTRUCTION OF SELF-INACTIVATING (SIN) FIV VECTORS

Self-inactivating (SIN) FIV vectors are made by altering or deleting promoter and enhancer elements in the U3 region of the 3′LTR to various degrees. SIN vectors have possible advantages for increased safety, the can decrease the chance of insertional activation of cellular genes and possibly provide tighter regulation of the gene of interest.

A. Construction of pTFIVL-SINΔ1611/1754

The FIV vector construct pTFIVL-SINΔ1611/1754 contains a 2 bp mutation in the TATA box and a 143 bp deletion NF-κB, AP-1 and ATF transcription factor binding sites within the U3 region of the 3′ FIV LTR.

The pTFIVL (Example 1B) vector will be used as the template to PCR amplify the 5′-half of the 3′ FIV LTR using the forward primer FIVNdelSense (SEQ ID No. 54; CCC TTT GAG GAA GGT ATG TCA TAT GAA TCC ATT TCG 5′) and the reverse primer FIVSINAlAnti(BglII) (SEQ ID No. 55; CGC GGA AGA TCT CAG GGT TCC AGT ACT CAT CCC AGT CCA CC). The resulting 267 bp PCR product is digested with Nde I and Bgl II. In parallel, the forward primers FIVSINΔ4Sense(BglI) (SEQ ID No.56; CGC GGA AGA TCT TAA GTT GTT CCA TTG TAA GAG TAT GCA ACC AGT GCT TTG TGA AAC TTC G) and the reverse primer FIVKpnIAntisense (SEQ ID No. 57; GGG AAC AAA AGC TGG GTA CCT GCG AAG TTC TCG G) is used to amplify the 3′-half of the FIV LTR, introducing a dinucleotide change in the TATA box motif (AT −>GC) leading to inactivation of the basal promoter. The resulting PCR fragment of 221 bp is digested with Bgl II and Kpn I.

Both digested PCR fragments and the Nde I/Kpn I digested (and phosphatased) pTFIVL vector are ligated in a three-way ligation, resulting in pTFIVL-SINΔ1611/1754.

B. Construction of pTFIVL-SINΔ1665/1754

The FIV vector construct pTFIVL-SINΔ1665/1754-contains two 2 bp mutations in the NF-KB and TATA box motifs, as well as an 89 bp deletion of the AP-1 and ATF sites within the U3 region 3′ FIV LTR.

The pTFIVL vector (Example 1B) will be used as the template to PCR amplify the 5′-half of the 3′ FIV LTR using the forward primer FIVNdelSense (SEQ ID. No 54; CCC TTT GAG GAA GGT ATG TCA TAT GAA TCC ATT TCG) and the reverse primer FIVSINΔ2Anti(BglII) (SEQ ID No. 58; CGC GGA AGA TCT TTT ATC ATT TGT TCG TAA ACA GTG GCT AGT CCA TAA GC). The resulting PCR product with 321 bp contains a dinucleotide change in the NF-cB transcription factor binding site (CC −>GG) and is digested with Nde I and Bgl II. The forward primer FIVSINΔ4Sense(BglI) (SEQ ID No. 56; CGC GGA AGA TCT TAA GTT GTT CCA TTG TAA GAG TAT GCA ACC AGT GCT TTG TGA AAC TTC G) and the reverse primer FIVKpnIAntisense (SEQ ID No. 57; GGG AAC AAA AGC TGG GTA CCT GCG AAG TTC TCG G) will be used to amplify the 3′-half of the FIV LTR, containing the dinucleotide change (mutation) in the TATA box motif The resulting 3′ LTR 221 bp PCR product is digested with Bgl II and Kpn I.

Both digested PCR fragments and the Nde I/Kpn I digested (and phosphatased) pTFIVL plasmid are ligated in a three-way ligation, resulting in pTFIVL-SINΔ1665/1754

C. Construction of pTFIVL-SINΔ1699/1754

The FIV vector pTFIVL-SINΔ1699/1754 (not underlined) contains site-specific mutations in the NF-κB, AP-4, AP-1, ATF and TATA box elements, as well as harbor a 55 bp deletion which removes the putative C/EBP and CAAT box binding sites. The pTFIVL-SINΔ1699/1754 construct has all but 61 bp (18%) of the wild type FIV 3′ LTR sequence, and should have no finctional candidate enhancer or promoter factor binding sites.

The pTFIVL vector will be used as the template to PCR amplify the 5′-half of the 3′ FIV LTR using the forward primer FIVNdeISense (SEQ ID No. 54; CCC TTT GAG GAA GGT ATG TCA TAT GAA TCC ATT TCG) and the reverse primer FIVSINΔ3Anti(BglII) (SEQ ID No. 59; CGC GGA AGA TCT CGC TTT AAC TAC AAG TCA TGC TCC ATA TTT CCT TTT ATC ATT TGT TCG TAA ACA GTG GCT AGT CCA TAA GC). The resulting 356 bp PCR fragment contains specific mutations in the following transcription factor binding sites: 1) a dinucleotide change in the NF-κB site, 2) a tri- to dinucleotide switch (AGC −>Calif.) in the AP-4 site, and 3) a dinucleotide change in the AP-1 site (TG −>Calif.). The PCR product is digested with Nde I and Bgl II and will be ligated into the Nde I/Bgl II digested pTFIVL-SINΔ1665/1754 (Example 15B) to construct the pTFIVL-SINΔ1699/1754 vector.

D. Construction of pVETT CB-SINΔ1611/1754

Following the completion of the intermediate FIV SIN vector pTFIVL-SINΔ1611/1754 (Example 15A) in the pTFIVL vector background, pTFIVL-SINΔ1611/1754 will be digested with Apa I and Kpn I and the liberated 718 bp fragment ligated into Apa I/Kpn I digested pTC/FLC, (Example 3N). The resulting pVET_{L}Cβ-SINΔ1611/1754 construct places the TATA box specific mutation and the 143 bp deletion (encompassing the NF-κB, AP-4, AP-1 and ATF sites) in the context of the hybrid FIV LTR vector with the β-galactosidase marker gene.

E. Construction of pVETT CD-SINΔ1665/1754

Following the completion of the intermediate FIV SIN vector pTFIVL-SINΔ1665/1754 (Example 15B) in the pTFIVL vector background, pTFIVL-SINΔ1665/1754 will be digested with Apa I and Kpn I and the liberated 718 bp fragment ligated into Apa I/Kpn I digested pTC/FLCβ (Example 3N). The resulting pVET_{L}Cβ-SINΔ1611/1754 construct places the TATA box, NF-cB and the 89 bp deletion (encompassing AP-4, AP-1 and ATF sites) mutations in the context of the hybrid FIV LTR vector with the β-galactosidase marker gene.

F. Construction of pVETT CB-SINΔ1699/1754

Following the completion of the intermediate FIV SIN vector pTFIVL-SINΔ1699/1754 (Example 15C) in the pTFIVL vector background, pTFIVL-SINΔ1699/1754 will be digested with Apa I and Kpn I and the liberated 718 bp fragment ligated into Apa I/Kpn I digested pTC/FLCβ (Example 3N). The resulting pVET_{L}Cβ-SINΔ1699/1754 construct contains site-specific mutations in the NF-κB, AP-4, AP-1, ATF and TATA box elements, as well as a 55 bp deletion which removes the putative C/EBP and CAAT box binding sites. The pVET_{L}CP-SINΔ1699/1754 construct has all but 18% of the wild type FIV 3′ LTR sequence and is in the context of the hybrid FIV LTR vector with the β-galactosidase marker gene.

### Example 17

### TITER EVALUATION OF Fiv SIN VECTORS

Self-inactivating (SIN) FIV vectors need to be evaluated for their titer potential since alterations in the 3′ FIV LTR may lead to reduced efficiencies of transduction of target cells as seen in the case of MLV-based retroviral SIN vectors ((Yu et al., Proc. Natl. Acad. Sci. 83: 3194, 1986; Yee et al., Proc. Natl. Acad. Sci. 84: 5197, 1987). FIV hybrid LTR vector pVET_{L}Cβ-SINΔ1699/1754 (Example 15F), pVET_{L}Cβ-SINΔ1611/1754 (Example 15E) or pVET_{L}Cβ-SINΔ1611/1754 (Example 15D) is transfected with a VSV-G expression cassette and the FIV gag/pol expression cassette pCFIVΔORF2ΔVIF (Example 5G) into 293T cells via CaPO4 transfection, viral supernatant harvested and titer determined as described in Example 8. In parallel, the positive control non-SIN hybrid FIV LTR vector pTC/FLC (Example 3N) is transfected into 293T cells as well and the titer used to give the 100% value for the evaluation of the SIN vector titers.

### Example 18

### CONSTRUCTION OF SPLrT GENOME FIV PACKAGING CONSTRUCTS

To generate FIV packaging constructs which encode the Gag and Pol polyproteins on separate plasmids, a Cla I site is first introduced into the FIV packaging construct pCFIVFRRE (Example 5H). The site is introduced at nt 2270 (ie. AATGTT to ACTGAT) using the PCR method described in Example 2A along with appropriate primers. The resulting construct, pCF FRRE/ClaI, is then digested with Cla I and Age I, treated with mung bean nuclease and religated to yield an FIV packaging construct containing the FIV RRE and coding only for the Gag polyprotein (pCFIVGag). To generate an FIV packaging construct coding only for the Pol polyprotein, a second Cla I site is introduced into pCFIVFRRE/ClaI at nt 640 (ie. CTCGAC to ATCGAT) using the PCR method described in Example 2A along with appropriate primers. The resulting construct, pCFIVFRRE/Cla2, is then digested with Cla I and the vector religated to yield an FIV packaging construct containing the FIV RRE and encoding only for the Pol polyprotein (pCFIVPol).

### Example 19

### GENERATION OF FIV MARKER RESCUE CELL LINES

Marker rescue cell lines for the detection of replication competent retroviruses (RCR) in FIV vector preparations are described in this Example. Briefly, the FIV virus establishes a productive infection in feline cells only. This is most likely due to at least two factors, 1) non-feline cells lack the main receptor for FIV and 2) the FIV LTR is less active in non-feline cells such as e.g. human cells. The co-receptor for FIV is identified as the CXCR4 molecule (Poeschla et al., J. Virol. 72:6858-6866, 1998; Willett et al., J ViroL 72:6475-6481, 1998). In order to circument the low acitivity of the FIV LTR in non-feline cells, a hybrid FIV LTR vector (Example 2) is introduced which is expected to increase the sensitivity of the detection assay. The following examples describe the production of marker rescue cell lines in human and feline cells.

A. Production of an FIV marker rescue cell line in HT1080 cells

It is unclear what kind of tropism an arising RCR in an FIV vector preparation would have but one can speculate that, by homologous and/or non-homologouse recombination of the RNA or DNA components, the RCR might have the tropism of the envelope provided in the FIV PCL (Example 19). Therefore, the RCR should be able to transduce a human cell line if the envelope is derived from the amphotropic MLV or VSV-G envelope.

HT1080 cells (ATCC, CCL-121) were co-transfected with the hybrid FIV LTR vector pVET_{L}Cb (Example 3N) and the phleomycin-resistant marker plasmnid pUT507. Forty-eight hours post-transfection, these cells were placed under zeocin selection at a concentration of 200ug/ml zeocin. The transfected pools were selected for 2 weeks and dilution cloned into 96-well plates using standard procedures to generate single-cell colony isolates. Dilution clones were analyzed for β-galactosidase expression by X-gal staining (Example 8) and D-gal positive clones were expanded and frozen.

Marker rescue cell clones are used for the marker rescue assay applying standard procedures for co-cultivation (Printz et al., Gene Therapy 2:143-150, 1995).

B. Production of an FIV marker rescue cell line in CrFK cells Marker rescue cell lines are produced in CrFK in case the RCR has the FIV-envelope. In that case, detection of the RCR is most sensitive if the correct FIV receptors are provided on the marker rescue line.

CrFK cells (ATCC, CCL-94) were co-transfected with the hybrid FIV LTR vector pVET_{L}Cb (Example 3N) and the phleomycin-resistant marker plasmid pUT507. Forty-eight hours post-transfection, these cells were placed under zeocin selection at a concentration of 150ug/ml zeocin. The transfected pools were selected for 2 weeks and dilution cloned into 96-well plates using standard procedures to generate single-cell colony isolates. Dilution clones were analyzed for β-galactosidase expression by X-gal staining (Example 8) and β-gal positive clones were expanded and frozen.

Marker rescue cell clones are used for the marker rescue assay essentially as described by Printz et al., Gene Therapy 2:143-150, 1995.

### Example 20

### GENERATION OF FIV PACKAGING CELL LINES

This Example describes the production of stable FIV packaging cell lines (PCL) based on parent cell lines from various species and cell types. The parent cell line includes various envelopes, FIV gag/pol and packaging expression cassettes, rev-expression cassettes, cassettes containing nucleic acids accommodating inducible and/or regulatable PCL components as well as plasmnids expressing selection markers. The order of introduction of the various components into the parent cell line to assemble a stable FIV PCL may vary. In general, components are added sequentially in order to decrease the risk of homologous recombination between the various components. A selection marker is introduced either within the expression cassette of a PCL component or on a separate plasmid that is co-transfected along with the expression cassette. The means of introduction of the various components into the parent cell lines may vary and includes all possible methods used for the introduction of nucleid acids into cells such as e.g. electroporation, CaPO4-transfection and cation-mediated transfection.

After transfection of an expression cassette and selection of the transfected pool, dilution cloning is carried out. A PCL intermediate clone which contains the introduced component is identified using the appropriate assays to confirm functionality of the introduced nucleic acid, before the next PCL component is introduced. This way, the PCL intermediates undergo several rounds of dilutional cloning and are sequentially transfected with various components to build a specific PCL clone.

The groups of PCL components introduced into parent cell lines are 1) packaging expression cassettes, 2) rev-expressing cassettes, 3) envelope expressing cassettes, 4) expression cassettes necessary to accommodate inducible/regulatable gene expression, 5) selection marker plasmids. Any combination of PCL components can be used to produce a stable FIV PCL but it should include a minimum of an FIV packaging expression cassette and an envelope expression cassette.

A. Production of human FIV PLC: Amphotropic

The following PCL components are sequentially introduced into the human HT1080 parent cell line (ATCC, CCL-121). Transfected are any one of the amphotropic envelope plasmnids such as pCMVenvAmDra/LBGH (described in PCT publication number WO 97/42338 entitled “Crossless Retroviral Vectors”) and the FWV packaging expression construct pCFIVΔORF2ΔVIF (Example 5G).

Briefly, the envelope cassette is co-transfected with a methotrexate marker plasmid into HTIO80. The standard CaPO4-precipitation procedure is applied using the Profectin kit (Promega Corp. Madison, Wis.) according to manufacturer's instructions using a 1:10 ratio of the marker: envelope plasmtid. 48 hours after the transfection, the methotrexate selection media is applied to the transfected cells (2×10⁻⁶ M dipyridamole, 2×10 methotrexate) and the pools selected for 2-3 weeks until all untransfected control cells are dead. The transfected and selected pool is dilution cloned according to standard procedures in 96-well plates. Single clones are isolated and analyzed for the expression of the envelope in a Western blot using anti-amphotropic envelope antibodies. Further assays for the intermediate PCL clones are fuinctionality assay such as e.g. transfecting an FIV gag/pol cassette and the pVET_{L}Cβ vector (Example 3N) into each clone. The top 4 PCL intermediate clones with the highest titer potential will be used for the introduction of the FIV gag/pol cassette. pCFIVΔORF2ΔVIF is co-transfected with a phleomycin-resistant marker into the four top clones as described above. Transfected pools are selected with zeocin (150 μg/ml) for 2 weeks and dilution cloned as described above. Single clones are tested for p24 expression using the PetCheck FIV Antigen Kit (IDEXX, Portland, Maine) and p24-positive clones subjected to a fuinctionality test. The FIV vector pVET_{L}Cβ is either transfected or transduced into the FIV PCL clones, the supernatant harvested 48 hours posttransfection (-transduction) and analyzed for blue colony forming foci as described in Example 8. PCL clones resulting in the highest titer output are used for further titer potential analysis with other FIV-based vectors and the top PCL clone with the highest titer potential will be the designated FIV PCL clone.

B. Production of human FIV PLC: Amphotropic, vif+

The following PCL components are sequentially introduced into the human HT1080 parent cell line (ATCC, CCL-121) as descnied above. Transfected are any one of the amphotropic envelope plasmids such as pCMVenvAmDra/LBGH (described in PCT publication number WO 97/42338 entitled “Crossless Retroviral Vectors”) and the FIV packaging expression construct pCFIVΔORF2 Example 5C).

C. Production of human FIV PLC: Amphotropic. orf2+

The following PCL components are sequentially introduced into the human HT1080 parent cell line (ATCC, CCL-121) as descnied above. Transfected are any one of the amphotropic envelope plasmids such as pCMVenvAmDraALBGH (described in PCT publication number WO 97/42338 entitled “Crossless Retroviral Vectors”) and the FIV packaging expression construct pCFIVΔVIF (Example 5F).

D. Production of human FIV PLC: Amphotropic. orf2+. vif+

The following PCL components are sequentially introduced into the human HT1080 parent cell line (ATCC, CCL-121) as described above. Transfected are any one of the amphotropic envelope plasmids such as pCMVenvAmDra/LBGH (described in PCT publication number WO 97/42338 entitled “Crossless Retroviral Vectors”) and the FIV packaging expression construct pCFIV (Example 5E).

E. Production of human FIV PLC: Xenotropic

The PCL production is carried out as described in Example 19A, except, that the xenotropic envelope pCMVX (described in PCT publication number WO 92/05266) is used instead of an amphotropic envelope.

F. Production of human FIV PLC: Xenotropic, vif+

The PCL production is carried out as described in Example 19B, except, that the xenotropic envelope pCMVX (described in PCT publication number WO 92/05266) is used instead of an amphotropic envelope.

G. Production of human FIV PLC: Xenotropic, orf2+

The PCL production is carried out as described in Example 19C, except, that the xenotropic envelope pCMVX (described in PCT publication number WO 92/05266) is used instead of an amphotropic envelope.

H. Production of human FIV PLC: Xenotropic, orf2+ , vif+

The PCL production is carried out as described in Example 19D, except, that the xenotropic envelope pCMVX (described in PCT publication number WO 92/05266) is used instead of an amphotropic envelope.

I. Production of human FIV PLC: GaLV envelope expressing

The PCL production is carried out as described in Example 19A-D, except, that the Gibbon Ape Leukemia Virus envelope (GaLV, Marybeth Eiden) is used instead of an amphotropic envelope.

J. Production of human FIV PLC: split gagipol cassette

The PCL production is carried out as described in Example 19A-I, except, that the FIV gag/pol expression cassette is split as descnied in Example 17.

K. Production of human FIV PLC: rev-deleted gagipol

The PCL production is carried out as described in Example 19A-I, except, that a rev-deleted FIV gag/pol expression cassette is used as described in Example 5H-L and additional HIV or FIV-based rev constructs are introduced where needed.

L. Production of human FIV PLC: inducible components

The PCL production is carried out as described in Example 19A-K, except, that one or more PCL components can be under an inducible/regulatable system as e.g. described in Example 21 using aptamers. Aptamers will be introduced into the FIV gag/pol expression cassette.

M. Production of human FIV PLC: inducible VSV-G

The PCL production is carried out as described in Example 19A-K, except, that the envelope is VSV-G which is under control under an inducible sytem.

N. Production of feline FIV PLC

The PCL production is carried out as described in Example 19A-M, except, that the parent cell line is the feline CrFK cell line (ATCC, CCL-94).

### Example 21

### PSEUDOTYPING OF FIV VECTORS

FIV-based vector particles may pseudotype with one or more heterologous viral envelopes or hybrid envelopes which are combinations of various viral envelopes sequences. Pseudotyping of FIV envelopes is analyzed by transfecting an FIV hybrid LTR vector with a marker gene, an FIV gag/pol construct and the envelope expression cassette under investigation into 293T cells via CaPO4 transfection. The supernatant will be harvested and the titer determined as described in Example 8. The titer determination is a measure for the pseudotyping activity and is compared with the titer of VSV-G pseudotyped FIV vectors. The VSV-G envelope efficiently pseudotypes FIV vectors (Example 9) and will be used as the positive control.

A. Pseudotyping of FW vectors with the amphotropic MLV envelope

The plasmid coding for the amphotropic envelope (MLV-derived 4070A) is called pCMVenvAmDralLBGH and is described in the PCT publication number WO 97/42338 entitled “Crossless Retroviral Vectors”. The hybrid FIV LTR vector PVET_{L}C13 (Example 3N), the FIV gag/pol packaging cassette pCFIVΔORF2ΔVIF (Example 5G) and pCMVenvAmDra/LBGH or the VSV-G envelope were transiently transfected into 293T cells, the supernatant harvested and the titer determined as described in Example 8. FIV vectors pseudotyped with the amphotropic envelope result in infectious viral particles which show an approximately one log lower transduction efficiency than VSV-G pseudotyped FIV vectors (Table 13).

TABLE 13
Pseudotyping efficiency of FIV vectors with the MLV
amphotropic envelope

| | | Pseudotyping |
|---|---|---|
| Envelope | Titer (CFU/ml) | efficiency (%)^{a} |
| Amphotropic | 1 × 10⁵ | 12.5 |
| VSV-G | 8 × 10⁵ | 100 |

^{a}The pseudotyping efficiency of FIV vectors with VSV-G is measured in titer (number of blue foci forming units/ml) and used as the 100% value. The pseudotyping efficiency of FIV vectors pseudotyped with envelopes other than VSV-G is adjusted accordingly.

B. Pseudotyping of FIV vectors with the xenotropic MLV envelope

The plasrmid coding for the xenotropic envelope NV-derived; NZB9-1 clone) is called pCMVxeno and is described in the PCT publication number WO 92/05266. The hybrid FIV LTR vector pVET_{L}Cβ (Example 3N), the FIV gag/pol packaging cassette pCFIVΔORF2ΔVIF (Example SG) and pCMVxeno or the VSV-G envelope are transiently transfected into 293T cells, the supernatant harvested and the titer determined as described in Example 8.

C. Pseudotvping of FIV vectors with the polytropic MLV envelope

The plasmid coding for the polytropic envelope (from clone MCF-247W) is called pCMVMCF and is described in the PCT publication number WO 92/05266. The hybrid FIV LTR vector pVET_{L}Cβ (Example 3N), the FIV gag/pol packaging cassette pCFIVΔORF2ΔVIF (Example 5G) and pCMVenvAmDra/LBGH or the VSV-G envelope are transiently transfected into 293T cells, the supernatant harvested and the titer determined as described in Example 8.

D. Pseudotyping of FIV vectors with the GaLV envelope

The Gibbon Ape Leukemia virus envelope (GaLV env) was cloned by Dr. Marybeth Eiden and is available through the NIH depository. The hybrid FIV LTR vector pVET_{L}Cβ (Example 3N), the FIV gag/pol packaging cassette pCFIVΔORF2ΔVIF (Example 5G) and the GaLV env or the VSV-G envelope are transiently transfected into 293T cells, the supernatant harvested and the titer determined as described in Example 8.

E. Pseudotypins of FIV vectors with the HIV-1 envelope

The hybrid FIV LTR vector pVET_{L}Cβ (Example 3N), the FIV gag/pol packaging cassette pCFIVΔORF2ΔVIF (Example 5G) and the HIV-1 envelope (Human Immunodeficiency Virus-1) or the VSV-G envelope are transiently transfected into 293 T cells, the supernatant harvested and the titer determined as described in Example 8.

F. Pseudotyping of FIV vectors with the SNV envelope

The hybrid FIV LTR vector pVET_{L}CP (Example 3N), the FIV gag/pol packaging cassette pCFIVΔORF2ΔVIF (Example 5G) and SNV env (Spleen Necrosis Virus) or the VSV-G envelope are transiently transfected into 293T cells, the supernatant harvested and the titer determined as described in Example 8.

### Example 22

### IN VIVO STUDES USING FIV VECTORS

FIV-based vector particles are introduced into different species animals via various routes into various organs for in vivo evaluation of FIV vectors. Parameters investigated are e.g. transduction efficiency, expression level of the gene of interest, duration of expression of the gene of interest, and ability to regulate gene expression of transgene in FIV vectors.

A. In vivo studies of FIV vectors coding for erythropoietin

An intravenous injection (i.v.) of a single dose of 200 μl FIV vector preparation 5 coding for Epo (erythropoietin) was injected into groups of 12 C57BL/6 mice. The Epo gene was driven by either an internal MLV LTR promoter (pVET_{L}MEpo, Example 3V) or an internal CMV promoter (pVET_{L}CEpo, Example 3X) in the context of an hybrid FIV LTR vector. Both transient FIV/Fpo vector preparations (pVET_{L}MEpo and pVET_{L}CEpo) were VSV-G pseudotyped and concentrated as described in Example 13A and B, respectively. A total of 4×10⁶ (pVET_{L}CEpo) and 2×10⁶ (PVET_{L}MEpO) infectious FIV/Epo vector particles were given per dose per animal. Both FIV/Epo vector preparations were free of mycoplasma and bacterial contamination and did not exceed endotoxin levels >10 units/dose. The control animals (group of 12) received 200 μl of saline. Epo expression levels were measured by analysis of Epo protein in serum using the Epo detection kit (Quantikine IVD, R&D Systems Inc., Minneapolis, Minn.). In addition, Epo expression was analyzed as a function of the increasing hematocrit. Epo protein expression levels and hematocrits in the three groups of mice were followed for 6 weeks and results are shown in Tables 14 and 15.

TABLE 14
Erythropoietin protein levels in mice over 6 weeks post-injection

| Injected | Mean of Epo protein for each group of mice (mIU/ml) | | | | | | |
|---|---|---|---|---|---|---|---|
| material | Injection | eek 1 | eek 2 | eek 3 | eek 4 | eek 5 | eek 6 |
| pVET_{L}CEpo- | | 9.1 | 2.4 | 3.7 | 3.9 | 0.8 | 9.8 |
| based | | | | | | | |
| FIV particles | | | | | | | |
| pVET_{L}MEpo- | | | .6 | .5 | .0 | .d. | .7 |
| based | | | | | | | |
| FIV particles | | | | | | | |
| Saline | | | | | | | |

TABLE 15
Hematocrits in mice over 8 weeks post-injection

| | Mean hematocrit for each group of mice (% PCV) | | | | | | | | |
|---|---|---|---|---|---|---|---|---|---|
| Injected | Injec- | eek | | | | | | | |
| material | tion | 1 | eek 2 | eek 3 | eek 4 | eek 5 | eek 6 | eek 7 | eek 8 |
| pVET_{L}C | 4-47 | 1 | 6 | 0 | 9 | 8 | 5 | 6 | 5 |
| Epo-based | | | | | | | | | |
| FIV | | | | | | | | | |
| particles | | | | | | | | | |
| pVET_{L}M | 4-47 | 5 | 1 | 3 | 6 | 8 | 7 | 5 | 6 |
| Epo-based | | | | | | | | | |
| FIV | | | | | | | | | |
| particles | | | | | | | | | |
| Saline | 4-47 | 4- | 4-47 | 4-47 | 4-47 | 4-47 | 4-47 | 4-47 | 4-47 |
| | | 47 | | | | | | | |

### Example 23

### REGT_{L}ATED GEN EXPRESSION

Within one embodiment of the invention, expression of structural or therapeutic gene products at the appropriate time, location and level can be accomplished utilizing one or more strategies which are designed to regulate either the expression of the specific gene, or the efficiency of translation of its RNA. Several examples are described which address regulation systems that manipulate the overall expression levels of a given gene product at either the transcriptional or translational level.

A. Use of aptamers: Construction of pFIVtheoAEpo

Since all vectors use host translational processes for protein expression, small molecules which bind to factors/proteins that mediate their effects at the transcriptional level can be utilized to regulate gene expression. This aspect of the invention utilizes a short RNA aptamer (SEQ ID No. 61; 5′-AGU GAU ACC AGC AUC GUC UUG AUG CCC UUG GCA GCA CU-3′) which is inserted into the the 5′-untranslated region of the Epo gene in the pVET_{L}MEpo vector (Example 3V), (Westuck and Green, Science 282: 296, 1998). This aptamer is designed to bind specifically and with high affinity to theophylline (Jenison et al., Science 263:1425, 1994), a soluble, cell permeable, non-toxic ligand, and will result in the translational control of Epo protein expression being specifically induced (regulated) following ligand treatment and binding.

Briefly, two complimentary primers (theoEcoRI sense, SEQ ID No. 62; AAT TCA GTG ATA CCA GCA TCG TCT TGA TGC CCT TGG CAG CAC TG; theoAvrII antisense, SEQ ID No. 63; TCA CTA TGG TCG TAG CAG AAC TAC GGG AAC CGT CGT GAG GAT C) are annealed and the double stranded synthetic DNA purified, kinased, and ligated into the EcoR I/Avr II digested pVET_{L}MEpo. This places the theophylline-specific aptamer within the 5′-untranslated region of the FIV-Epo vector.

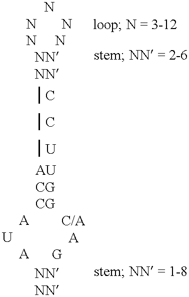

high affinity aptamer for theophylline: (SEQ ID No. 64)

From the foregoing it will be appreciated that, although specific embodiments of the invention have been described herein for purposes of illustration, various modifications may be made without deviating from the spirit and scope of the invention. Accordingly, the invention is not limited except as by the appended claims.

TABLE OF CONTENTS

| Construction of FIV vector constructs | | | 7 |
|---|---|---|---|
| Construction of packaging expression cassettes | | | 13 |
| Construction of envelope (env) expression cassettes | | | 15 |
| Packaging/Producer Cell Lines | | | 17 |
| Genes of Interest/Heterologous Nucleic Acid Molecules | | | 20 |
| Regulated Expression of Structural and Therapeutic Gene Products | | | 34 |
| Methods for Utilizing FIV Vector Particles | | | 38 |
| | 1. | Immunostimulation | 39 |
| | 2. | Blocking Agents | 42 |
| | 3. | Expression of Palliatives | 44 |

| | | |
|---|---|---|
| a. | Inhibitor Palliatives | 44 |
| b. | Conditional Toxic Palliatives | 46 |

| | | |
|---|---|---|
| 4. | Expression of Markers | 49 |
| 5. | Immune Down-Regulation | 50 |
| 6. | Replacement or Augmentation Gene Therapy | 51 |

| | | |
|---|---|---|
| a. | Treatment of Gaucher disease | 51 |
| b. | FIV vector particles Expressing Human Factor | 52 |
| | VIII and Factor IX for Treatment of | |
| | Hemophilia | |
| c. | FIV vector particles expressing other clotting | 58 |
| | factors | |
| d. | FIV vector particles treatment of hereditary | 59 |
| | disorders and other conditions | |

| | | |
|---|---|---|
| 7. | Lymphokines and Lymphokine Receptors | 62 |

| | | |
|---|---|---|
| a. | FIN vector particles for treatment of viral | 64 |
| | hepatitis | |

| | 8. | Suicide Vectors | 66 |
|---|---|---|---|
| | 9. | FIN vectors to Prevent the Spread of Metastatic | 66 |
| | | Tumors | |
| | 10. | Modulation of Transcription Factor Activity | 67 |
| | 11. | FIN vector particle delivery to cats | 68 |
| Formulation | | | 69 |
| Administration | | | 74 |
| EXAMPLE 1 | | | 79 |
| | Construction of FIV Vectors | | 79 |
| | A. | Construction of the pTFIVS vector | 80 |
| | B. | Construction of the pTFIVL vector | 81 |
| EXAMPLE 2 | | | 82 |
| | Construction of hybrid FIV LTR Vectors | | 82 |
| | A. | Construction of the pTC/FIVS hybrid FIV LTR vector | 82 |
| | B. | Construction of the pTC/FIVL hybrid FIV LTR vector | 83 |
| EXAMPLE 3 | | | 83 |
| | Insertion of Promoter/Reporter Gene Cassettes Into | | 83 |
| | FIV vectors | | |
| | A. | Generation of the pCMVβgal expression cassette | 84 |
| | B. | Generation of the pCMVβgalCTE expression cassette | 84 |
| | C. | Generation of the pCMVβgalPRE expression cassette | 84 |
| | D. | Generation of the pCMVβgalRRE expression cassette | 85 |
| | E. | Construction of the pTFSCβ FIV vector | 85 |
| | F. | Construction of the pTFLCβ FIV vector | 85 |
| | G. | Construction of the pTFSCβCTE FIV vector | 85 |
| | H. | Construction of the pTFLCβCTE FIV vector | 86 |
| | I. | Construction of the pTFSCβPRE FIV vector | 86 |
| | J. | Construction of the pTFLCβPRE FIV vector | 86 |
| | K. | Construction of the pTFSCβRRE FIV vector | 86 |
| | L. | Construction of the pTFLCβRRE FIV vector | 86 |
| | M. | Construction of the pTC/FSCβ hybrid FIV LTR vector | 87 |
| | | (pVET_{S}Cβ) | |
| | N. | Construction of the pTC/FLCβ hybrid FIV LTR vector | 87 |
| | | (pVET_{L}Cβ) | |
| | O. | Construction of the pTC/FLCβCTE hybrid FIV LTR | 87 |
| | | vector (pVET_{L}CβCTE) | |
| | P. | Construction of the pTC/FLCLuc hybrid FIV LTR | 87 |
| | | vector (pVET_{L}CLuc) | |
| | Q. | Construction of the pTC/FLCeGFP hybrid FIV LTR | 87 |
| | | vector (pVET_{L}CeGFP) | |
| | R. | Construction of the pTC/FLMeGFP hybrid FIV LTR | 88 |
| | | vector (pVET_{L}MeGFP) | |
| | S. | Construction of FIV vectors containing an RRE | 88 |
| | | upstream of the promoter/reporter gene cassette | |
| | T. | Construction of the pTC/FLRβgluc hybrid FIV LTR | 89 |
| | | vector (pVET_{L}Rβgluc) | |
| | U. | Construction of the pTC/FLMCFTR hybrid FIV LTR | 90 |
| | | vector (pVET_{L}MCFTR) | |
| | V. | Construction of the pTC/FLMEpo hybrid FIV LTR | 90 |
| | | vector (pVET_{L}MEpo) | |
| | W. | Construction of the pTC/FLMEpo-short hybrid FIV | 91 |
| | | LTR vector (pVET_{L}MEpo(Δ5′)) | |
| | X. | Construction of the pTC/FLCEpo hybrid FIV LTR | 91 |
| | | vector (pVET_{L}CEpo) | |
| | Y. | Construction of the pTC/FLPEpo hybrid FIV LTR | 92 |
| | | vector (pVET_{L}PEpo) | |
| | Z. | Construction of the pTC/FLC16K-Ser hybrid FIV LTR | 92 |
| | | vector (pVET_{L}C16K-Ser) | |
| | AA. | Construction of the pTC/FLM16K-Ser hybrid FIV | 93 |
| | | LTR vector (pVET_{L}ML6K-Ser) | |
| | AB. | Construction of the pTC/FLChGH hybrid FIV LTR | 94 |
| | | vector (pVET_{L}ChGH) | |
| EXAMPLE 4 | | | 94 |
| | Insertion of Reporter Gene Cassettes Into FIV Vectors | | 94 |
| | A. | Construction of the pTFSβ FIV vector | 94 |
| | B. | Construction of the pTFLβ FIV vector | 95 |
| | C. | Construction of the pTC/FLEpo hybrid FIV LTR | 95 |
| | | vector (pVET_{L}Epo) | |
| EXAMPLE 5 | | | 95 |
| | Construction of FIV Packaging Expression Cassettes | | 95 |
| | A. | Construction of Packaging Expression Cassette, | 96 |
| | | pCMVFIVXho (pCFIVX) | |
| | B. | Construction of Packaging Expression Cassette, | 97 |
| | | pCMVFIVSal | |
| | C. | Construction of Packaging Expression Cassette, | 97 |
| | | pCMVFIVΔ17S (pCFIVΔORF2) | |
| | D. | Construction of Packaging Expression Cassette, | 98 |
| | | pCMVFIVSalΔ17 | |
| | E. | Construction of Packaging Expression Cassette, pCFIV | 98 |
| | F. | Construction of Packing Expression Cassette, | 99 |
| | | pCFIVΔVIF | |
| | G. | Construction of Packaging Expression Cassette, | 99 |
| | | pCFIVΔORF2ΔVIF | |
| | H. | Construction of FIV rev Deletion Packaging | 99 |
| | | Expression Cassettes | |
| | I. | Construction of Packaging Expression Cassette, | 100 |
| | | pCFIVΔSDFRRE | |
| | J. | Construction of Packaging Expression Cassette, | 100 |
| | | pCFIVΔSDHRRE | |
| | K. | Construction of Packaging Expression Cassette, | 101 |
| | | pCFIVΔSD4XCTE | |
| | L. | Construction of Packaging Expression Cassette, | 101 |
| | | pCFIVΔSDg/p | |
| EXAMPLE 6 | | | 102 |
| | Production of Pseudotyped FIV Particles | | 102 |
| EXAMPLE 7 | | | 102 |
| | Infection of Cultured Cells by Pseudotyped FIV Particles | | 102 |
| EXAMPLE 8 | | | 103 |
| | Production of FIV Vector Particles | | 103 |
| EXAMPLE 9 | | | 104 |
| | Determination of FIV Vector Construct Requirements | | 104 |
| EXAMPLE 10 | | | 106 |
| | Determination of FF1 Packaging Construct Requirement | | 106 |
| EXAMPLE 11 | | | 108 |
| | Requirement for rev/RRE in FIV Vector and Packaging | | |
| | | Constructs | 108 |
| EXAMPLE 12 | | | 111 |
| | Infection of Non-Proliferating Cells By FIV Vector Particles | | 111 |
| | A. | Transduction of Primary Human Skin Fibroblasts | 111 |
| | B. | Transduction of Growth-Arrested HT1080 Cells | 112 |
| | C. | Transduction of Quiescent CD4 + T lymphocytes | 114 |
| | D. | Transduction of Terminally Differentiated Human | 115 |
| | | Macrophages | |
| EXAMPLE 13 | | | 117 |
| | Transductiuion of Hematopoietic Progenitor Cells | | 117 |
| | A. | Transduction of Mouse Hematopoietic Progenitor Cells | 117 |
| | | (HPC) | |
| | B. | Transduction of Human Hematopoietic Progenitor | 118 |
| | | Cells (HPC) | |
| EXAMPLE 14 | | | 119 |
| | FIV Vector Particle Purification and Concentration | | 119 |
| | A. | Concentration of pVET_{L}ChGH and pVET_{L}MEpo FIV | 120 |
| | | vector particles | 120 |
| | B. | Concentration of pVET_{L}MEpo FIV vector particles | 121 |
| | C. | Concentration of pVET_{L}CEpo vector | 122 |
| EXAMPLE 15 | | | 123 |
| | Construction of FIV Vectors With Minimal Packaging Signal | | 123 |
| | A. | Construction of pVET_{S}Cβ(740) | 124 |
| | B. | Construction of pVET_{S}Cβ(627) | 124 |
| | C. | Construction of pVET_{S}Cβ(540) | 124 |
| | D. | Construction of pVET_{S}Cβ(360) | 125 |
| EXAMPLE 16 | | | 125 |
| | Construction Of Self-Inactivating (SIN) FIV Vectors | | 125 |
| | A. | Construction of pTFIVL-SINΔ1611/1754 | 125 |
| | B. | Construction of pTFIVL-SINΔ1665/1754 | 126 |
| | C. | Construction of pTFIVL-SINΔ1699/1754 | 126 |
| | D. | Construction of pVET_{L}Cβ-SINβ1611/1754 | 127 |
| | E. | Construction of pVET_{L}Cβ-SINβ1665/1754 | 127 |
| | F. | Construction of pVET_{L}Cβ-SINβ1699/1754 | 128 |
| EXAMPLE 17 | | | 128 |
| | Titer Evaluation of Fiv SIN Vectors | | 128 |
| EXAMPLE 18 | | | 129 |
| | Construction of Split Genome FIV Packaging Constructs | | 129 |
| EXAMPLE 19 | | | 129 |
| | Generation of FIV Marker Rescue Cell Lines | | 129 |
| EXAMPLE 20 | | | 131 |
| | Generation of FIV Packaging Cell Lines | | 131 |
| EXAMPLE 21 | | | 135 |
| | Pseudotyping of FIV Vectors | | 135 |
| | A. | Pseudotyping of FIV vectors with the | 135 |
| | | amphotropic MLV envelope | |
| | B. | Pseudotyping of FIV vectors with the xenotropic MLV | 136 |
| | | envelope | |
| | C. | Pseudotyping of FIV vectors with the polytropic MLV | 136 |
| | | envelope | |
| | D. | Pseudotyping of FIV vectors with the GaLV envelope | 137 |
| | E. | Pseudotyping of FIV vectors with the HIV-1 envelope | 137 |
| | F. | Pseudotyping of FIV vectors with the SNV envelope | 137 |
| EXAMPLE 22 | | | 137 |
| | In Vivo Studies Using FIV Vectors | | 137 |
| | A. | In vivo studies of FIV vectors coding | 138 |
| | | for erythropoietin | |

| EXAMPLE 23 | | 140 |
|---|---|---|
| | Regulated Gene Expression | 140 |

1. A method for transducing hematopoietic progenitor cells comprising:
providing a hematopoietic progenitor cell;
contacting said hematopoietic progenitor cells with an FIV vector particle, said FIV vector particle having a 5′ FIV LTR, a primer binding site, one or more heterologous sequences(s) operably linked to a promoter element, an origin of second strand DNA synthesis and a 3′ FIV LTR, allowing said FIV vector particle to be imported into said hematopoietic progenitor cell; and
expressing said one or more heterologous sequences in said hematopoietic progenitor cell.

2. The method according to claim 1 further comprising:
isolating said hematopoietic progenitor cell from a mammal's hematopoietic tissues selected from the group consisting of, bone marrow, peripheral blood, unbiblical cord blood, or visceral organs.

3. The method according to claim 2 when in said isolating method is selected from the group consisting of differential centrifugation, immunoaffinity techniques, fluorescent-activated cell sorting techniques, and combinations therefor.

4. The method according to claim 2 wherein said mammal is a human.

5. An FIV vector for transfortning mammalian hematopoietic progenitor cells comprising:
5′ FIV LTR, a primer binding site, one or more heterologous sequences(s) operably linked to a promoter element, an origin of second strand DNA synthesis and a 3′ FIV LTR.

6. The FIV vector according to claim 5 wherein said primer-binding site is a tRNA binding site.

7. The FIV vector according to claim 5, further comprising a packaging signal.

8. The FIV vector of claim 5 wherein the FIV vector contains less than 20 consecutive nucleotides occurring within a gag/pol or env sequence of a retrovirus.

9. The FIV vector of claim 5 wherein the FIV vector contains less than 15 consecutive nucleotides occurring within a gag/pol or env sequence of a retrovirus.

10. The FIV vector of claim 5 wherein the FIV vector contains less than 10 consecutive nucleotides occurring within a gag/pol or env sequence of a retrovirus.

11. The FIV vector of claim 5 wherein the FIV vector contains less than 6 consecutive nucleotides occurring within a gag/pol or env sequence of a retrovirus.

12. The FIV vector according to claim 5 wherein the promoter element is a tissue-specific promoter.

13. The FIV vector according to claim 5 wherein the promoter element is a viral promoter.

14. The FIV vector according to claim 5 wherein the viral promoter is a CMV, SV40, PGK or HIV-1 promoter.

15. The vector according to claim 5 wherein at least one of the 5′ FIV LTR, the 3′ FIV LTR or the FIV LTR promoter is comprised of at least 45% of a wild type sequence.

16. The vector according to claim 5, further comprising at least one non-FIV promoter or promoter/enhancer.

17. The vector according to claim 16 wherein said promoter or promoter/enhancer is inducible.

18. The FIV vector according to claim 5 wherein at least one gene of interest is a marker gene.

19. The FIV vector according to claim 5 wherein at least one gene of interest is selected from a gene encoding cytokines, factor VIII, factor IX, LDL receptor, prodrug activating enzymes, trans-dominant negative viral or cancer-associated proteins, angiogenesis and anti-angiogenesis factors, CFTR, β-glucuronidase, sarcoglycans, glucokinase, NGF, VEGF, PDGF, IGF, GC, BDNF, and tyrosine hydroxylase.

20. A method for making FIV vector particles for transforming mammalian hematopoietic progenitor cells comprising, introducing into a host cell an FIV vector construct, and one or more expression cassettes that direct the expression of FIV gag, FIV pol and a viral envelope.

21. The method according to claim 20 wherein said viral envelope is a VSV-G envelope.
